(19) United States
(12) Patent Application Publication
(10) Pub. No.: US 2013/0130312 A1
(43) Pub. Date: May 23, 2013

(54) METHODS AND STRAINS FOR THE PRODUCTION OF SARCINAXANTHIN AND DERIVATIVES THEREOF

(71) Applicants: Roman Netzer, Trondheim (DE); Trygve Brautaset, Trondheim (DE); Per Bruheim, Trondheim (DE)

(72) Inventors: Roman Netzer, Trondheim (DE); Trygve Brautaset, Trondheim (DE); Per Bruheim, Trondheim (DE)

(73) Assignee: Promar AS, Fornebu (NO)

(21) Appl. No.: 13/701,344

(22) PCT Filed: Jun. 1, 2011
(86) PCT No.: PCT/EP2011/059159

§ 371 Date: Feb. 6, 2013

(30) Foreign Application Priority Data
Jun. 2, 2010 (GB) 1009269.0

(51) Int. Cl. C12P 23/00; C12N 1/20

(52) U.S. Cl. CPC C12P 23/00; C12N 1/20
USPC 435/67; 536/23.2; 435/320.1; 435/189; 435/252.1

(57) ABSTRACT

The present invention relates to a new strain of Micrococcus luteus, named Otnes7, which is superior to known strains in its ability to synthesise the carotenoid sarcinaxanthin and a method of producing sarcinaxanthin or a derivative thereof, said method comprising introducing into and expressing in a host cell one or more nucleic acid molecules encoding an activity in the sarcinaxanthin biosynthetic pathway.

The present invention relates to a new strain of Micrococcus luteus, named Otnes7, which is superior to known strains in its ability to synthesise the carotenoid sarcinaxanthin. The invention also relates to the identification and cloning of the gene cluster encoding the biosynthetic machinery for the synthesis of sarcinaxanthin, which includes the first known proteins responsible for the biosynthesis of a γ-cyclic C₅₀ carotenoid and more particularly the identification for the first time of a C₅₀ carotenoid γ-cyclase. In particular, novel genes and their encoded polypeptides from the novel Otnes7 strain are identified and sequenced. The invention accordingly provides the novel nucleic acid molecules and proteins from said strain. The invention further relates to the use of nucleic acid molecules encoding the sarcinaxanthin biosynthetic machinery enzyme system (as well as components thereof) in methods for the production of sarcinaxanthin, through heterologous expression of said nucleic acids and proteins in host cells.

Pigmentation is widespread among bacteria and pigments found in marine heterotrophic bacteria comprise carotenoid, flexirubin, xanthomonadine and prodigiosin (Kim et al., 2007; Reichenbach et al., 1980). The carotenoids are considered to be the main and most abundant pigment group.

Carotenoids are natural pigments synthesized by bacteria, fungi, algae and plants and to date more than 750 different natural carotenoids have been isolated from natural sources. In addition to their importance as coloration pigments, carotenoids play a critical role in photosynthetic processes and exhibit protective properties against damage by oxygen and light. Due to their antioxidant properties, carotenoids have been proposed to reduce the risk of certain cancers, cardiovascular disease and Alzheimer's disease. The global market for carotenoids used as food colourants and nutritional supplements was estimated at some $935 million by 2005 (Fraser and Bramley 2004). Despite intensive research into microbial production of carotenoids, most commercial carotenoids are still produced by chemical synthesis and only large-scale microbial production of β-carotene (Raja, Hemaiswarya et al. 2007) and astaxanthin (Fang and Cheng 1992) has been reported to date. There is an increasing demand for natural carotenoids for nutritional, pharmaceutical and medical applications, and hence the microbial production of these molecules is of great importance.

More than 95% of all natural carotenoids are based on a symmetric C₄₀ phytoene backbone and only a small number of C₃₀ and even fewer C₅₀ carotenoids have been discovered so far. Carotenoids modified by oxygen-containing functional groups are cyclic or acyclic xanthophylls which have been shown completely to lack pro-oxidative abilities and display significant stronger anti-oxidative properties than carotenoids without oxygen functionality (carotenes). The extension of conjugated double bonds has also been reported to increase the anti-oxidative potential of hydroxylated carotenoids and is assumed as one of the most important features for radical scavenging properties. Based on the high number of conjugated double bonds, and since all known C₅₀ carotenoids contain at least one hydroxyl group, this class of carotenoids has a high potential for excellent anti-oxidative properties. Thus there is interest in the production of carotenoids in this class.

In nature C₅₀ carotenoids are synthesized by bacteria of the actinomycetales family. The ε-cyclic C₅₀ carotenoid decaprenoxanthin (2,2′-Bis-(4-hydroxy-3-methylbut-2-enyl)-ε,ε-carotene) has been found in Agromyces mediolanus, Arthrobacter glacialis and Aureobacterium sp., and the decaprenoxanthin biosynthetic pathway was proposed in Corynebacterium glutamicum (Krubasik and Sandmann 2000; Krubasik, Kobayashi et al. 2001). The β-cyclic C₅₀ carotenoid C.p. 450 (2,2′-Bis-(4-hydroxy-3-methylbut-2-enyl)-β,β-carotene) has been detected in Curtobacterium flaccumfaciens (formerly Corynebacterium poinsettiae) and recently the biosynthetic pathway in Dietzia sp. CQ4 was proposed (Tao, Yao et al. 2007). For both C₅₀ carotenoid pathways it was reported that the common precursor lycopene is synthesized via the methylerythritol 4-phosphate (MEP) pathway which is present in most eubacteria (Rodriguez-Concepcion and Boronat 2002). Biosynthesis of lycopene from C₁₅ farnesyl pyrophosphate (FPP) has been well studied in many carotenogenic organisms. FPP is converted into C₂₀ geranyl geranyl pyrophosphate (GGPP) catalyzed by GGPP synthase, followed by condensation of two molecules GGPP to produce C₄₀ phytoene, catalyzed by a phytoene synthase. Finally, phytoene is dehydrated to C₄₀ lycopene, catalyzed by a phytoene dehydrogenase. Heterologous production of lycopene has been performed successfully in non-carotenogenic organisms such as Escherichia coli and is being investigated intensively on an ongoing basis (Das, Yoon et al. 2007).

Using lycopene as the precursor, biosynthesis of cyclic C₅₀ carotenoids is catalyzed by lycopene elongase and carotenoid cyclases. Although most carotenoids in plants and microorganisms exhibit cyclic structures, cyclization reactions are predominantly known for C₄₀ pathways, catalyzed by monomeric enzymes which have been isolated from plants and bacteria. In C. glutamicum, the genes crtYe, crtYf and crtEb were identified to be involved in the conversion of lycopene to the ε-cyclic C₅₀ carotenoid decaprenoxanthin. Sequential elongation of lycopene by two C₅ isoprenyl units to form the acyclic C₅₀ carotenoid flavuxanthin was catalyzed by a crtEb encoded lycopene elongase. Subsequent cyclization to decaprenoxanthin was catalyzed by a heterodimeric C₅₀ carotenoid ε-cyclase encoded by crtYe and crtYf. Whilst the polypeptides encoded by crtYe and crtYf share primary sequence similarities with a new type of the heterodimeric lycopene cyclase CrtYc and CrtYd involved in lycopene cyclization in B. linens and Mycobacterium aurum, the C. glutamicum crtYeYf genes encode two polypeptides constituting a carotenoid cyclase that uses C₄₅ and C₅₀ carotenoids as substrates (Krubasik, Kobayashi et al. 2001). The genetic and enzymatic basis for glycosylation of decaprenoxanthin in C. glutamicum is unknown.

Recently, an analogous pathway was proposed for the biosynthesis of the β-cyclic C₅₀ carotenoid C.p. 450 in Dietzia sp. CQ4 (Tao, Yao et al. 2007). Synthesis of C.p. 450 from lycopene also requires lycopene elongase and C₅₀ carotenoid β-cyclase activity.

Whilst most cyclic carotenoids exhibit β-rings, &ring containing pigments are common in higher plants. Carotenoids substituted only with γ-rings are rarely observed in plants and algae, and only traces can be detected. Prior to the present invention, no biochemical pathway for γ-cyclic C₅₀ carotenoids had been identified.

Sarcinaxanthin is a γ-cyclic C₅₀ carotenoid which is known to be produced by Micrococcus luteus. Micrococcus luteus is a GC rich Gram-positive bacterium belonging to the family of micrococcaceae within the order of actinomycetales. The carotenoids, including sarcinaxanthin, accumulated in this bacterium were identified and structurally elucidated decades ago. However, the biosynthetic machinery responsible for the synthesis of this molecule was, prior to the present invention, unknown. As suggested above, the elucidation and functional characterization of the genes responsible for the biosynthesis of the γ-cyclic C₅₀ carotenoid sarcinaxanthin and its glycosylated derivatives is of great commercial importance and represents a significant contribution to knowledge in the biosynthesis of carotenoids. As discussed below, this has resulted in a much needed advance in methods for the production of sarcinaxanthin and the identification of a new class of cyclase, namely a C₅₀ carotenoid γ-cyclase, which will be useful in the synthesis of structurally different carotenoids.

As noted above and described below, the present invention is based on the identification, cloning and sequencing of a gene cluster for the biosynthesis of sarcinaxanthin which has not heretofore been available. Furthermore, the present inventors have isolated a novel strain of M. luteus, named Otnes7, which is capable of producing sarcinaxanthin in superior quantities to other known strains. The identification, cloning and sequencing of the gene cluster for the biosynthesis of sarcinaxanthin from M. luteus strain NCTC2665 has allowed the identification and cloning of nucleic acids from the Otnes7 strain, which encode novel proteins the expression of which results in increased sarcinaxanthin production in comparison to the proteins of the NCTC2665 strain. Heterologous expression of one or more of the sarcinaxanthin biosynthesis genes in a host cell has enabled a method for efficiently and economically producing sarcinaxanthin.

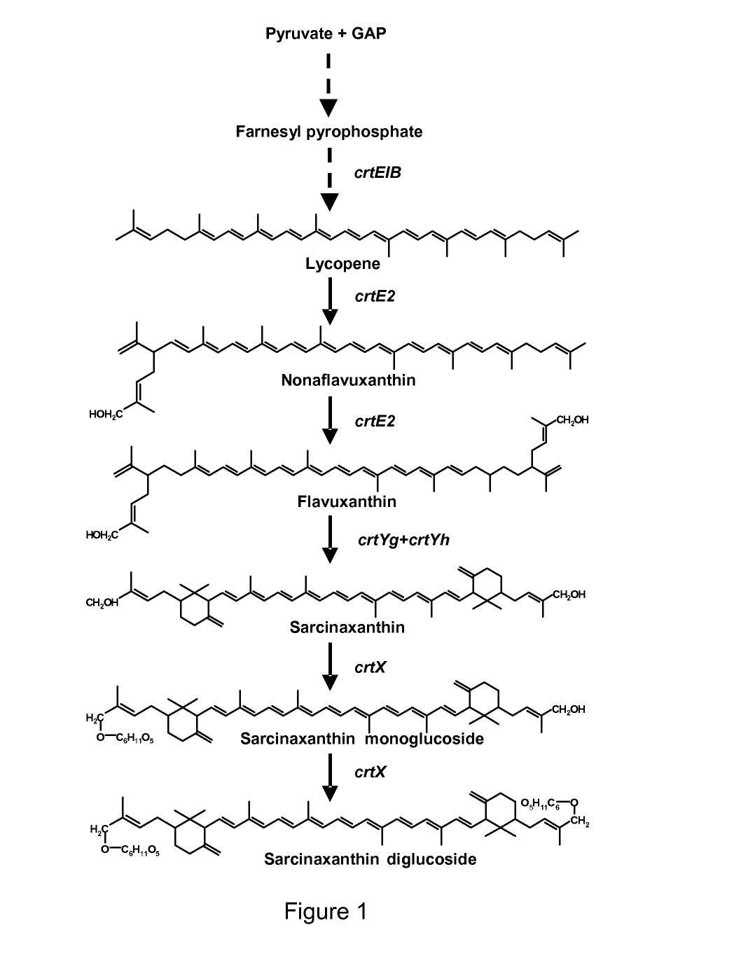

Analysis of the cloned genes has further allowed the elucidation of the biosynthetic pathway for sarcinaxanthin. Accordingly it is now proposed that the normal process of synthesis of sarcinaxanthin is initiated through the synthesis of lycopene, as described above, which is converted to nonaflavuxanthin and then flavuxanthin through the action of a lycopene elongase, which in M. luteus is encoded by the gene crtE2. The resultant flavuxanthin is cyclised by the action of a heterodimeric C₅₀ γ-cyclase, which in M. luteus is encoded by crtYg and crtYh, which results in sarcinaxanthin (FIG. 1). The sacrinaxanthin biosynthetic gene cluster also encodes at least one protein (CrtX) for the glycosylation of the synthesized molecules.

Since the chemical synthesis of compounds such as this is highly complex, a biosynthetic route in practice needs to be used and accordingly the isolation or purification of the compounds from appropriate hosts, particularly heterologous hosts (that is hosts transformed with one or more genes to enable the biosynthesis), is desirable. This also affords the opportunity of manipulating genes of the biosynthetic gene cluster in order to change the biosynthesis and thereby result in improved yields and/or the synthesis of new or modified carotenoid compounds.

In this respect, there remains a need and desire to provide methods for the improved production of carotenoid compounds (for example to improve yield, or production conditions, or to expand the range of available host cells) and the present invention is directed to these aims, based on the cloning and DNA sequencing of the sarcinaxanthin biosynthetic gene cluster. This provides the first characterisation for these carotenoid biosynthetic genes, as well as a tool for genetic manipulation in order to modify the expression levels or properties of sarcinaxanthin and/or the producing organism. Whilst the carotenoid sarcinaxanthin is known and the sequence of the genome of M. luteus strain NCTC2665 is available, in view of the background of a plurality of carotenoid-based molecules synthesised in M. luteus and the corresponding plurality of biosynthetic genes necessary for their synthesis, and further in view of the relatively poor sequence homology between the sequences of the present invention and the known carotenoid biosynthesis genes, it was not a straightforward matter to identify and clone the sarcinaxanthin gene cluster; a considerable effort and ingenuity in terms of sequence analysis was required. Furthermore, only after the identification and characterisation of the sarcinaxanthin gene cluster from M. luteus strain NCTC2665 was it possible to identify homologous genes from the novel Otnes7 strain of the invention, which as discussed below resulted in the identification of genes the expression of which resulted in improved efficiency of sarcinaxathin production over the genes of the NCTC2665 strain.

The present inventors have isolated and purified sarcinaxanthin from a previously unknown source, bacterial isolate Otnes7, believed to be a novel strain of M. luteus (deposited in the name of the applicant under the deposit number DSM 23579, on 29 Apr. 2010, at the Deutsche Sammlung von Mikroorganismen and Zellkulturen GmbH (DSMZ)) which was isolated from the surface micro layer of the mid-part of the Norwegian coast. The isolation of this novel microorganism has enabled the inventors to clone and sequence a novel sarcinaxanthin biosynthetic gene cluster, which shows improved activity in comparison to known strains. The biosynthetic gene cluster contains 8 genes that encode proteins that are believed to be involved in the biosynthesis of the sarcinaxanthin molecule and derivatives thereof (see Table 1).

Based on the knowledge of the sequence, the inventors have been able to use various methods of genetic manipulation to confirm the activity of the proteins encoded by the gene cluster and to show that the sequences identified in the Otnes7 strain are indeed responsible for enhanced sarcinaxanthin biosynthesis.

The complete coding sequence for (i.e. the complete nucleotide sequence encoding) the sarcinoxanthin biosynthetic gene cluster from the NCTC2665 strain is shown in SEQ ID NO. 1. This has been shown to contain a number of genes or ORFs, that are believed to encode all of the proteins and polypeptides that are required for normal sarcinaxanthin biosynthesis in M. luteus. The group of proteins and polypeptides encoded by the gene cluster as a whole are collectively referred to as the biosynthetic machinery for the biosynthesis of sarcinaxanthin.

In silico screening the of the M. luteus strain NCTC2665 DNA sequence data (which has been deposited under accession number NC_{—}012803) resulted in the initial identification of a putative carotenoid biosynthesis gene cluster consisting of six open reading frames, or1009-or1014 (comprised within SEQ ID NO: 1). The deduced or1014 gene product displayed only 31% and 33% primary sequence identity to known CrtE proteins of C. glutamicum and Dietzia sp., respectively, both encoding geranyl geranyl pyrophosphate (GGPP) synthases. CrtE catalyzes the first reaction specific to the carotenoid branch of general isoprenoid metabolism, the conversion of farnesyl pyrophosphate (FPP) into GGPP. The or1014 gene was therefore designated crtE (SEQ ID NO: 18 and 19). The deduced or1013 gene product displayed only 41% and 48% primary sequence identity to the CrtB proteins of C. glutamicum and Dietzia sp., respectively, which are phytoene synthases which catalyze the condensation of two GGPP molecules to phytoene. The or1013 gene was therefore designated crtB (SEQ ID NO: 20 and 21). The deduced or1012 gene product displayed only 43% and 53% primary sequence identity to the CrtI proteins of C. glutamicum and Dietzia sp., respectively. These proteins are phytoene desaturases which catalyse conversion of phytoene to lycopene by stepwise desaturation reactions. The or1012 gene was therefore designated crtI (SEQ ID NO: 22 and 23). The deduced or1011 gene product displayed only 50% and 52% primary sequence identity to the lycopene elongases in C. glutamicum and in Dietzia sp., respectively. In C. glutamicum this enzyme (encoded by crtEb) catalyses the conversion of lycopene into nonaflavuxanthin and flavuxanthin. Secondary structure analysis revealed six transmembrane helices for the M. luteus elongase, five for the C. glutamicum elongase and eight for the Dietzia sp. elongase, strongly indicating that all are transmembrane proteins. The or1011 gene was designated crtE2 (SEQ ID NO: 6 and 8). The deduced or1010 and or1009 gene products displayed only 32% and 31% primary sequence identity to the C₅₀ ε-cyclase subunits in C. glutamicum encoded by crtYe and crtYf, respectively. They also shared only 36% and 38% primary sequence identity to the corresponding proteins in Dietzia sp. In C. glutamicum, the crtYe and crtYf gene products are small polypeptides assumed to form a heterodimeric enzyme that catalyses the conversion of flavuxanthin into decaprenoxanthin. Both gene products exhibit three transmembrane helices. Secondary structure analysis revealed also three transmembrane helices for each C₅₀ cyclase subunit from C. glutamicum and Dietzia sp. The or1010 and or1009 genes were designated crtYg (SEQ ID NO: 2 and 3) and crtYh (SEQ ID NO: 4 and 5), respectively.

Further analysis of the gene cluster revealed that immediately downstream of crtYh there is a an ORF encoding a hypothetical protein (SEQ ID NO: 24 and 25), followed by or1007 which encodes a putative polypeptide sharing only 43% sequence identity to the putative glycosyl transferase protein CrtX from Dietzia sp., suggested to be involved in the glycosylation of C.p. 450 (Tao, Yao et al. 2007). The or1007 gene was therefore designated crtX (SEQ ID NO: 16 and 17).

Without wishing to be bound by any single hypothesis, it is believed, due to the proximal localization and similar orientation of the genes, that the crtEIBE2YgYh genes are cotranscribed in M. luteus. Moreover, the assumed stop codons of crtB, crtI, crtE2 and crtYg overlap the start codon of the corresponding subsequent gene which may allow translational coupling to ensure equimolar expression and/or proper folding of the products. Whilst the genetic organization of crt genes in M. luteus displays some similarities to the previously published biosynthetic gene clusters for the C₅₀ carotenoids C.p. 450 and decaprenoxanthin in Dietzia sp., in view of the differences in the order of the genes and the relatively low sequence identity between the genes it was only after experimental analysis, as discussed elsewhere herein, that the above described gene cluster was confirmed as being involved in sarcinaxanthin biosynthesis.

As discussed above, the sarcinaxanthin biosynthetic gene cluster is a nucleic acid molecule which contains the various genetic elements or different genes or ORFs that encode the proteins or polypeptides that are required for the biosynthesis of the sarcinaxanthin molecule or a sarcinaxanthin derivative. However, not all of the encoded proteins and polypeptides have yet been ascribed a role in the biosynthesis and so it is thought that not all of the encoded proteins or polypeptides of the cluster are essential for sarcinaxanthin biosynthesis. The various genes and ORFs may encode enzymes that catalyse one or more biochemical reactions, or proteins that do not have catalytic activity but instead are involved in other processes such as the regulation of the process of sarcinaxanthin synthesis, or sarxinaxanthin transport, for example.

Each sarcinaxanthin biosynthetic gene or ORF encodes a single polypeptide chain (which can alternatively be described as a protein; the terms “polypeptide” and “protein” are used interchangeably herein) that has or is believed to have a function in the biosynthesis of the sarcinaxanthin molecule or a derivative thereof. Eight such genes or ORFs have been identified (see Table 1). As shown in FIG. 1, six of these are ascribed a direct role in the biosynthesis of sarcinaxanthin, whilst a seventh has been shown to have a role in the glycosylation of sarcinaxanthin to mono- and diglucoside forms and the eighth has not yet been ascribed a function.

However, as discussed further below, only two of the genes or ORFs are essential for the biosynthesis of sarcinaxanthin, i.e. those encoding the enzyme which catalyses the final step of the biosynthetic pathway that results in the conversion of flavuxanthin to sarcinaxanthin (namely crtYg and crtYh) and the other genes may be replaced by genes encoding enzymes with equivalent functional activities, or alternative activities that result in the production of flavuxanthin, i.e. the substrate for the C₅₀ carotenoid γ-cyclase encoded by said genes. In other words, for the production of sarcinaxanthin in a host cell it is not necessary to introduce into said cell the entire biosynthetic cluster from M. luteus (although this is contemplated by the present invention) as the introduction of genes encoding the enzymes that catalyse the final step in the biosynthetic pathway is sufficient for the production of sarcinaxanthin as long as the substrate for the sarcinxanthin-synthesising C₅₀ carotenoid γ-cyclase, i.e. flavuxanthin, is present in said cell.

In particular, as described in the examples herein, it has been found that higher levels of sarcinaxanthin production may be obtained by recombinant expression of the sarcinaxanthin-producing enzymes (i.e. of the sarcinaxanthin biosynthetic machinery) in a heterologous host, as compared with sarcinaxanthin production in native M. luteus cells. Thus, in terms of sarcinaxanthin production, recombinant expression is favoured over extraction from natural sources (i.e. over isolation of the product from cells in which it is naturally produced).

Thus in a very general sense, the present invention provides a method of producing sarcinaxanthin or a derivative thereof, said method comprising introducing into and expressing in a host cell one or more nucleic acid molecules encoding the sarcinaxanthin biosynthetic pathway.

By allowing the nucleic acid molecules to be expressed, the encoded biosynthetic machinery may act in the host cell to synthesise the sarcinaxanthin, which may be recovered from the host cell. Thus, in the method above, the sarcinaxanthin or derivative thereof is synthesised in the host cell, and the method may comprise the further step of isolating the sarcinaxanthin or derivative thereof from the host cell.

As noted above, it is not necessary to introduce the entire biosynthetic pathway into the host, as long as the host is capable of making an intermediate, or substrate in the pathway (i.e. a sarcinaxanthin precursor). For example, a host already capable of synthesising lycopene, and/or flavuxanthin, may be used.

Thus, in a further broad sense, the invention may be seen as providing a method of producing sarcinaxanthin or a derivative thereof, said method comprising introducing into and expressing in a host cell one or more nucleic acid molecules encoding an activity in the sarcinaxanthin biosynthetic pathway.

As noted above, such a host cell will be a cell which produces an appropriate substrate or substrates for the introduced activity or activities, for example a lycopene-producing host cell, or a flavuxanthin-producing host cell. Preferably the host cells do not endogenously contain all of the nucleic acid molecules required for the synthesis of sarcinaxanthin or a derivative thereof, i.e. do not naturally produce sarcinaxanthin, but may preferably comprise nucleic acid molecules encoding proteins required for the synthesis of sarcinaxanthin precursors, e.g. lycopene, nonaflavuxanthin or flavuxanthin. Such nucleic acid molecules may be present endogenously i.e. the host cell may be a native producer of lycopene, nonaflavuxanthin and/or flavuxanthin. In a particularly preferred embodiment the host cell is a cell or microorganism other than that from which the nucleic acid molecules were (or from which they may be) derived and in which the molecules are natively present.

As will be described in more detail below, the nucleic acid molecules which are introduced will preferably encode one or more of the biosynthetic proteins of the organism M. luteus. In other words the nucleic acid molecules will be derived from, or will correspond to, the crt genes of M. luteus, as described herein. As noted above, and described in more detail below, in certain cases, for example in case of proteins involved in the biosynthesis up to the intermediate flavuxanthin, nucleic acid molecules encoding equivalent proteins from other sources may be used.

More particularly, the method of the invention involves (or comprises) the introduction and expression of a nucleic acid molecule encoding a protein having C₅₀ carotenoid γ-cyclase activity. Such a protein may be an enzyme which catalyses the conversion of flavuxanthin to sarcinaxanthin, and in particular such an enzyme which performs this reaction in M. luteus. Thus, the protein may correspond to the gene product of the crtYgYh genes of M. luteus. Such proteins are described further below.

As noted above, the gene cluster for the entire biosynthetic pathway for sarcinaxanthin has been cloned and identified in M. luteus. Whilst a nucleic acid molecule corresponding to the entire gene cluster of M. luteus may be used according to the invention, nucleic acid molecules based on genes encoding equivalent proteins from other sources may be used to provide the host cell with the proteins needed to synthesize a substrate, or intermediate, in the pathway. Thus for example host cells producing lycopene are known in the art, as are nucleic acid molecules encoding lycopene-synthesising enzymes, which may be used to engineer a host cell suitable for use according to the invention, to produce lycopene. Similarly a flavuxanthin-producing host cell may be used, or may be engineered to produce flavuxanthin.

Accordingly, one aspect of the invention thus provides a method of producing sarcinaxanthin or a derivative thereof, said method comprising introducing into and expressing in a host cell:

(a) one or more nucleic acid molecules comprising nucleotide sequences encoding one or more proteins capable of synthesising flavuxanthin; and

(b) one or more nucleic acid molecules comprising nucleotide sequences encoding one or more proteins having or contributing to C₅₀ carotenoid γ-cyclase activity, for example proteins capable of catalysing the conversion of flavuxanthin to sarcinaxanthin.

A further, more particular, aspect of the invention thus provides a method of producing sarcinaxanthin or a derivative thereof, said method comprising introducing into and expressing in a lycopene-producing host cell:

(a) one or more nucleic acid molecules comprising nucleotide sequences encoding one or more proteins capable of catalysing the conversion of lycopene to flavuxanthin, or, alternatively viewed, having lycopene elongase activity; and

(b) one or more nucleic acid molecules comprising nucleotide sequences encoding one or more proteins having or contributing to C₅₀ carotenoid γ-cyclase activity, or, alternatively viewed, capable of catalysing the conversion of flavuxanthin to sarcinaxanthin.

In the context above the term “contributing” is meant to reflect that the C₅₀ carotenoid γ-cyclase enzyme is heterodimeric, and that on its own a single subunit, e.g. as encoded by crtYg or crtYh alone, is not active—both subunits are required for the C₅₀ carotenoid γ-cyclase activity, but a single subunit contributes to activity.

More specific embodiments of these aspects of the invention are described further below. However, in general terms nucleic acid molecules of (b) may be obtained or derived from M. luteus, e.g. they may correspond to or be derived from the nucleotide sequences from M. luteus encoding proteins having or contributing to C₅₀ carotenoid γ-cyclase activity, as described herein, more particularly they may be correspond to or be derived from the crtYg or crtYh genes of M. luteus as described herein. The nucleic acid molecules encoding proteins capable of synthesising flavuxanthin may be obtained or derived from other sources, for example from genes known to be efficient in encoding proteins for lycopene synthesis in other organisms (e.g. the crtEIB genes from Pantoea ananatis, which are particularly useful in this respect, are described below), and by way of further example, nucleic acid molecules encoding proteins having lycopene elongase activity may be obtained or derived from organisms synthesising flavuxanthin, such as Corynebacterium glutamicum (crtEb) or from M. luteus (crtE2).

Thus, more particularly the method of the invention may involve introducing into and expressing in a host cell one or more nucleic acid molecules comprising a nucleotide sequence encoding:

(i) a protein capable of catalysing the conversion of farnesyl pyrophosphate (FPP) into geranyl geranyl pyrophosphate (GGPP) (e.g. a protein as encoded by a crtE gene);

(ii) a protein capable of catalysing the condensation of GGPP to phytoene (e.g. a protein as encoded by a crtB gene);

(iii) a protein capable of catalysing the conversion of phytoene to lycopene, or alternatively put a protein having phytoene dehydrogenase activity (e.g. a protein as encoded by a crtI gene);

(iv) a protein capable of catalysing the conversion of lycopene to flavuxanthin, or, alternatively viewed, having lycopene elongase activity (e.g. a protein as encoded by a crtE2 or a crtEb gene); and

(v) a protein having or contributing to C₅₀ carotenoid γ-cyclase activity, or, alternatively viewed, capable of catalysing the conversion of flavuxanthin to sarcinaxanthin (e.g. proteins as encoded by a crtYg gene and a crtYh gene as described herein).

As noted above, in a preferred embodiment nucleic acid molecules encoding (iv) and (v) above are introduced into a lycopene-producing host.

However, it is not precluded that the invention comprises the introduction of all the activities (i) to (v) set out above, and this may depend on the selected host, particular nucleic acid molecules involved etc. Thus, by way of representative example only, the method of the invention may comprise introducing into a host cell and expressing a nucleic acid molecule comprising the nucleotide sequence encoding the entire biosynthetic gene cluster, for example as obtained or derivable from a strain of M. luteus, e.g. as set forth in SEQ ID NO: 1, SEQ ID NO: 26 or SEQ ID NO: 37, or a sequence with at least 70% sequence identity to SEQ ID NO: 1, 26 or 37, or a part thereof, including particularly a part encoding the sarcinaxanthin biosynthetic pathway. In further embodiments, such a molecule may include a part of SEQ ID NO: 1, 26 or 37 which encodes one or more activities in the biosynthetic pathway, and more particularly a part which encodes a C₅₀ carotenoid γ-cyclase activity.

The nucleic acid molecule(s) which are introduced may be in the form of a single nucleic acid molecule or separate nucleic acid molecules. Thus a single nucleic acid molecule may comprise nucleotide sequences encoding all of the proteins/activities which are to be introduced, or the proteins/activities may be encoded by nucleotide sequences provided by (or on) more than one nucleic acid molecule.

The nucleic acid molecules for use in the method of the invention need not comprise the entire sarcinaxanthin biosynthetic gene cluster but may comprise a portion or part of it, more specifically a part encoding one or more proteins having a particular enzymic activity, and particularly a C₅₀ carotenoid γ-cyclase activity, more particularly a lycopene elongase activity and a C₅₀ carotenoid γ-cyclase activity.

A “sarcinaxanthin biosynthetic gene or ORF” refers to a gene or ORF which encodes a protein or polypeptide that is functional in the biosynthetic process of sarcinaxanthin or a sarcinaxanthin derivative. As noted above, this could be an enzyme that is involved in any step of the pathway, not only the final step of conversion of flavuxanthin to sarcinaxanthin, but also in the synthesis of lycopene or flavuxanthin or the precursors thereof, a protein that is involved in the modification of sarcinaxanthin to produce a sarcinaxanthin derivative (e.g. a glycosylated derivative) or a protein that is required for regulation or for transport of the molecule at any stage of its biosynthesis.

A nucleic acid molecule of the invention and for use in the method of the invention may be an isolated nucleic acid molecule (in other words isolated or separated from the components with which it is normally found in nature) or it may be a recombinant or a synthetic nucleic acid molecule.

The nucleic acid molecules may encode (or comprise a nucleotide sequence encoding) at least 1, or more, e.g. 2, 3, 4, 5, 6, 7 or 8 of the polypeptides or proteins that are involved in the biosynthesis of the sarcinaxanthin or a sarcinaxanthin derivative. For example, the method may involve the introduction of a single nucleic acid molecule encoding, e.g. proteins having lycopene elongase and C₅₀ carotenoid γ-cyclase activity, for example crtE2, crtYh and crtYg (or proteins with the equivalent functional activity, e.g. crtEb in place of crtE2). Alternatively it may comprise nucleic acid molecules corresponding to all of the ORFs/genes as set out in Table 1 except any one or more of crtX and the gene encoding the hypothetical protein (ORF1).

Each of the nucleic acid molecules of the method of the invention thus encodes one or more polypeptides involved in the biosynthesis of, or having functional activity in, the synthesis of sarcinaxanthin or a sarcinaxanthin derivative. Such a molecule may encode not only the known proteins, as they are found in nature, but also a functionally equivalent variant of a such a native protein, that is a protein which retains the activity of the native protein, which comprises one or more modifications in its amino acid sequence, for example an amino acid substitution, deletion, and/or insertion. Thus, fragments (or parts) of proteins are included as long as they retain the activity of the parent protein. Furthermore, also included are degenerate nucleic acid molecules, i.e. nucleic acid molecules in which the nucleotide sequence is varied with respect to the native sequence, but which encodes the same polypeptide. As defined above, the nucleic acid molecules of the invention may thus comprise functionally equivalent variants of SEQ ID NO: 1, SEQ ID NO: 26 or SEQ ID NO: 37 and such variants may include parts, degenerate sequences, or homologues defined by a % sequence identity to SEQ ID NO. 1. Such functionally equivalent variants encode proteins/polypeptides having functional activity as defined above. Furthermore, “parts” or “portions” as described herein may be functional equivalents. Preferably these portions satisfy the identity (relative to a comparable region) or hybridizing conditions mentioned herein.

Such functional activity may be enzymatic activity e.g. an activity involved in the synthesis of sarcinaxanthin. Such activities, or proteins having such activities are as defined above, and may be e.g. an activity corresponding to the activity of crtE, crtB, crtI, crtE2, crtYg and/or crtYh. Such functional activity may also be sarcinaxanthin glycosylase activity corresponding to the activity of crtX.

As mentioned above, a number of genes and ORFs have been identified within SEQ ID NO: 1, SEQ ID NO: 26 and SEQ ID NO: 37 and parts or fragments which correspond to such genes or ORFs represent preferred “parts” or fragments of SEQ ID NO: 1, 26 or 37. These are tabulated in Table 1 below:

TABLE 1

| | | | | SEQ ID NO: |
|---|---|---|---|---|
| | Start position | End position | | (nucleic |
| | in SEQ ID | in SEQ ID | Function of | acid/ |
| Name | NO: 1 (bp) | NO: 1 (bp) | encoded protein | protein) |
| crtE | 561 | 1637 | Geranyl geranyl | 18/19 |
| | | | pyrophosphatase | |
| | | | (GGPP) | |
| crtB | 1639 | 2535 | Phytoene synthase | 20/21 |
| crtI | 2532 | 4232 | Phytoene desaturase | 22/23 |
| crtE2 | 4229 | 5113 | Lycopene elongase | 6/8 |
| crtYg | 5110 | 5472 | C₅₀ γ-cyclase | 2/3 |
| | | | subunit | |
| crtYh | 5469 | 5822 | C₅₀ γ-cyclase | 4/5 |
| | | | subunit | |
| ORF1 | 5767 | 6375 | Hypothetical protein | 24/25 |
| crtX | 6372 | 7163 | Sarcinaxanthin | 16/17 |
| | | | glycosylase | |
| | | | | SEQ ID NO: |
| | Start position | End position | | (nucleic |
| | in SEQ ID | in SEQ ID | Function of | acid/ |
| Name | NO: 26 (bp) | NO: 26 (bp) | encoded protein | protein) |
| crtE | 1 | 1077 | Geranyl geranyl | 27/28 |
| | | | pyrophosphatase | |
| | | | (GGPP) | |
| crtB | 1079 | 1975 | Phytoene synthase | 29/30 |
| crtI | 1972 | 3672 | Phytoene desaturase | 31/32 |
| crtE2 | 3669 | 4553 | Lycopene elongase | 10/11 |
| crtYg | 4550 | 4912 | C₅₀ γ-cyclase | 12/13 |
| | | | subunit | |
| crtYh | 4909 | 5265 | C₅₀ γ-cyclase | 14/15 |
| | | | subunit | |
| | | | | SEQ ID NO: |
| | Start position | End position | | (nucleic |
| | in SEQ ID | in SEQ ID | Function of | acid/ |
| Name | NO: 37 (bp) | NO: 37 (bp) | encoded protein | protein) |
| crtE | 1 | 1077 | Geranyl geranyl | 27/28 |
| | | | pyrophosphatase | |
| | | | (GGPP) | |
| crtB | 1079 | 1975 | Phytoene synthase | 29/30 |
| crtI | 1972 | 3672 | Phytoene desaturase | 31/32 |
| crtE2 | 3669 | 4553 | Lycopene elongase | 10/11 |
| crtYg | 4550 | 4912 | C₅₀ γ-cyclase | 12/13 |
| | | | subunit | |
| crtYh | 4909 | 5265 | C₅₀ γ-cyclase | 14/15 |
| | | | subunit | |
| ORF1 | 5210 | 5818 | Hypothetical protein | 35/36 |
| crtX | 5815 | 6606 | Sarcinaxanthin | 33/34 |
| | | | glycosylase | |

As described in more detail below, further work has revealed the presence of additional genes within the gene cluster which is represented by SEQ ID NO:26. Thus, although not shown in SEQ ID NO:26, this gene cluster also includes a crtX gene, encoding a sarcinaxanthin glycosylase, the nucleotide and encoded amino acid sequences of which respectively are shown in SEQ ID NOs: 33 and 34. The “full length” gene cluster of the Otnes 7 strain is shown in SEQ ID NO: 37.

The sequences set out above thus represent sarcinaxanthin biosynthetic genes or ORFs. In other words, such genes/ORFs are found within the sarcinaxanthin biosynthetic gene cluster and encode proteins or polypeptides which have or are proposed to have a role in the biosynthesis of sarcinaxanthin in M. luteus. The term “sarcinaxanthin biosynthetic gene” or “sarcinaxanthin biosynthetic ORF” also includes genes and ORFs which encode proteins that share activity or function with the above proteins, and for example share high levels of sequence identity, as discussed elsewhere herein. They can alternatively be described as “functionally equivalent variants” or “functional equivalents”.

In this respect, the sarcinaxanthin biosynthetic gene cluster has also been cloned from the novel Micrococcus luteus strain Otnes7, and the proteins encoded by said genes can be considered as functional equivalents of the NCTC2665 sarcinaxanthin biosynthetic proteins. However, as discussed elsewhere herein, the Otnes7 strain produces increased levels of carotenoids in comparison to the NCTC2665 strain, e.g. 190 μg/g cell dry weight (CDW) and 145 μg/g CDW, respectively. This difference in sarcinaxanthin production is sufficient to distinguish between the two strains by visual inspection as the difference between colour intensities of the M. luteus strains demonstrates clearly that the Otnes7 strain produces higher levels of sarcinaxanthin than the NCTC2665 strain. Furthermore, when expressed in a heterologous host, the Otnes7 genes resulted in higher sarcinaxanthin production levels as compared to expression of the NCTC2665 genes. From experimental analysis of the Otnes7 biosynthetic gene cluster the present inventors were able to determine that the Otnes7 genes comprise specific sequence modifications as compared to the genes from the NCTC2665 strain. It is unclear exactly why the Otnes7 genes result in increased production, and this may depend upon the host used for the expression. However, it is possible that they encode proteins which have an enhanced catalytic activity (or substrate conversion efficiency) in comparison to genes of the NCTC2665 strain. Specifically, in the experiments in the examples described below the CrtE2 protein from the Otnes7 strain shows a relative conversion efficiency of lycopene to nonaflavuxanthin and flavuxanthin of 79% in comparison to the equivalent protein from the NCTC2665 strain, which has a conversion efficiency of only 23%. Furthermore, when the nucleic acids from the Otnes7 strain encoding CrtE2, CrtYg and CrtYh are expressed in a heterologous host cell, at least 97% of the carotenoid produced was sarcinaxanthin, wherein the expression of the same genes from NCTC2665 resulted in only about 90% of the carotenoids produced being sarcinaxanthin.

Thus, in a further, and preferred, aspect the present invention also provides nucleic acid molecules which correspond to, or are based on or derived from, the Otnes7 genes (i.e. the sarcinaxanthin biosynthetic gene cluster of the Otnes7 strain).

In one embodiment of this aspect the invention can be seen to provide a nucleic acid molecule comprising or consisting of all or a part of a nucleotide sequence as set forth in SEQ ID NO: 26 or 37 or which has at least 90% sequence identity to SEQ ID NO. 26 or 37, which molecule encodes one or more proteins having activity in the biosynthesis of sarcinaxanthin, and wherein any nucleic acid molecule which comprises a nucleotide sequence which is a part of SEQ ID NO. 26 or 37 or which is at least 90% identical to SEQ ID NO. 26 or 37 encodes proteins which are able to synthesise sarcinaxanthin at substantially the same level as the proteins encoded by SEQ ID NO: 26 or 37 when expressed in a host cell.

Thus, such a nucleic acid molecule encoding a part of SEQ ID NO: 26 or 37 or a variant of SEQ ID NO: 26 or 37 or a part thereof which variant has at least 90% sequence identity, may encode a particular protein or enzyme in the pathway, or a protein which is a constituent part of a enzyme in the pathway. When such a nucleic acid molecule is expressed, for example with other nucleic acid molecules corresponding to parts of SEQ ID NO: 26 or 37 encoding other enzymes/proteins in the pathway, the level of sarcinaxanthin production is substantially the same as when SEQ ID NO: 26 or 37 is expressed in the host cell. In other words, a sequence-variant or a part of SEQ ID NO: 26 or 37 will encode an activity, or a protein contributing to an activity which is at the same or an equivalent level to the activity of the protein encoded by SEQ ID NO: 26 or 37. “Substantially the same level” may be taken to mean activity which is at least 90%, more particularly at least 91, 92, 93 or 94%, more preferably at least 95, 96, 97, 98 or 99% of the activity of the equivalent protein encoded by SEQ ID NO: 26 or 37. Thus the nucleic acid molecules of the invention encode proteins which are substantially as active as the native proteins encoded by SEQ ID NO: 26 or 37 i.e. they retain the improved properties of the Otnes7 genes.

It will be evident from the structure of the sarcinaxanthin biosynthetic gene cluster from M. luteus NCTC2665 described above, that the sarcinaxanthin biosynthetic gene cluster from the Otnes 7 strain may comprise also encoding sequences in addition to those presented in SEQ ID NO: 26, i.e. the encoding sequences presented in SEQ ID NO: 37. For instance, the sarcinaxanthin biosynthetic gene cluster from the Otnes 7 strain also comprises a nucleic acid region encoding a protein with sarcinaxanthin glycosylase activity, i.e. a crtX gene. Hence, the present invention may also be seen to provide a nucleic acid molecule comprising or consisting of all or a part of a nucleotide sequence as set forth in SEQ ID NO: 37 or which has at least 90% sequence identity to SEQ ID NO. 37, which molecule encodes one or more proteins having activity in the biosynthesis of sarcinaxanthin, and wherein any nucleic acid molecule which comprises a nucleotide sequence which is a part of SEQ ID NO. 37 or which is at least 90% identical to SEQ ID NO. 37 encodes proteins which are able to synthesise sarcinaxanthin at substantially the same level as the proteins encoded by SEQ ID NO: 37 when expressed in a host cell.

In a preferred aspect of the invention the nucleic acid molecule comprises or consists of all or a part of a nucleotide sequence as set forth in SEQ ID NO: 26 or which has at least 90% sequence identity to SEQ ID NO. 26, which molecule encodes one or more proteins having activity in the biosynthesis of sarcinaxanthin, and wherein any nucleic acid molecule which comprises a nucleotide sequence which is a part of SEQ ID NO. 26 or which is at least 90% identical to SEQ ID NO. 26 encodes proteins which are able to synthesise sarcinaxanthin at substantially the same level as the proteins encoded by SEQ ID NO: 26 when expressed in a host cell.

More particularly, the present invention also provides a nucleic acid molecule comprising (or consisting of) a nucleotide sequence encoding all or part of a protein having an amino acid sequence as set forth in SEQ ID NO: 11 or an amino acid sequence which is at least 90% identical to SEQ ID NO: 11 and wherein said nucleotide sequence encodes a lycopene elongase with a lycopene to flavuxanthin conversion efficiency of at least 30%, when expressed in a host cell, or a nucleic acid molecule which comprises a nucleotide sequence which is the complement of any aforesaid sequence.

Preferably, the conversion efficiency is at least 40, 50, 60, 70, 75 or 80%.

A nucleic acid molecule as defined in this aspect of the invention may comprise or consist of:
(i) a nucleotide sequence as set forth in SEQ ID NO: 10;
(ii) a nucleotide sequence which is degenerate with the sequence of SEQ ID NO: 10;
(iii) a nucleotide sequence which has at least 90% sequence identity to SEQ ID NO: 10;
(iv) a nucleotide sequence which is a part of the nucleotide sequence of SEQ ID NO: 10 or of a nucleotide sequence which is degenerate therewith; or
(v) a nucleotide sequence which is complementary to any of (i) to (iv) above.

Additionally the present invention provides a nucleic acid molecule comprising (or consisting of) a nucleotide sequence encoding all or part of a protein having an amino acid sequence selected from the sequences as set forth in any one of SEQ ID NO: 11, 13 and 15 or an amino acid sequence which has at least 90% sequence identity to SEQ ID NO: 11, 13 or 15, and wherein said nucleotide sequence encodes a protein which when expressed in a lycopene-producing host cell together with each of the other said proteins results in at least 91% of the total carotenoids produced being sarcinaxanthin, or a nucleic acid molecule which comprises a nucleotide sequence which is the complement of any aforesaid sequence.

Preferably, at least 92, 93, 94, 95, 96, 97, 98 or 99% of the total carotenoids produced is sarcinaxanthin.

Furthermore, the present invention provides a nucleic acid molecule comprising (or consisting of) a nucleotide sequence encoding all or part of a protein having an amino acid sequence selected from the sequences as set forth in any one of SEQ ID NO: 11, 13 and 15 or an amino acid sequence which has at least 90% sequence identity to SEQ ID NO: 11, 13 or 15, wherein said nucleotide sequence encodes a protein which when expressed in a lycopene-producing host cell together with each of the other said proteins results in sarcinaxanthin production to a level of at least 150 μg/g of cell dry weight (CDW).

Preferably, sarcinaxanthin is produced to a level of at least 300, 500, 750, 1000, 2000, 2500 μg/g CDW.

More particularly, in these aspects of the invention as set out above, the protein of SEQ ID NO: 11 or of a part or sequence variant thereof has lycopene elongase activity and the proteins of SEQ ID NOs: 13 and 15 or parts or sequence variants thereof have or contribute to C₅₀ carotenoid γ-cyclase activity (e.g. together have C₅₀ carotenoid γ-cyclase activity) or more particularly are capable of catalysing the conversion of flavuxanthin to sarcinaxanthin.

Included within these aspects of the invention is a nucleic acid molecule comprising or consisting of:
(i) a nucleotide sequence selected from sequences as set forth in SEQ ID NO: 10, 12 and 14;
(ii) a nucleotide sequence which is degenerate with the sequence of any one of SEQ ID NOs: 10, 12 or 14;
(iii) a nucleotide sequence which has at least 90% sequence identity to any one of SEQ ID NOs: 10, 12 or 14;
(iv) a nucleotide sequence which is a part of the nucleotide sequence of any one of SEQ ID NOs: 10, 12 or 14 or of a nucleotide sequence which is degenerate therewith; or
(v) a nucleotide sequence which is complementary to any of (i) to (iv) above.

Alternatively or additionally the present invention also provides a nucleic acid molecule comprising (or consisting of) a nucleotide sequence encoding a protein having lycopene elongase activity and an amino acid sequence as set forth in all or part of SEQ ID NO: 11 or an amino acid sequence which is at least 90% identical to SEQ ID NO: 11, wherein said amino acid sequence comprises one or more of the following:
(a) alanine at position 8;
(b) valine at position 88;
(c) valine at position 158;
   or a nucleotide sequence which is the complement of any aforesaid sequence.

The position numbers are stated with reference to SEQ ID NO. 11.

Preferably the nucleic acid encodes a lycopene elongase with a conversion efficiency, or which enables sarcinaxanthin production, as defined above. More preferably the nucleic acid molecule comprises a nucleotide sequence as set forth in SEQ ID NO: 10 or a part of variant thereof as defined above, or a complement thereof.

Similarly, the invention provides a nucleic acid molecule comprising (or consisting of) a nucleotide sequence encoding a protein which contributes to (or more particularly which is a subunit of a protein having) C₅₀ carotenoid γ-cyclase activity and which has an amino acid sequence as set forth in all or part of SEQ ID NO: 13 or an amino acid sequence which is at least 90% identical to SEQ ID NO: 13, wherein said amino acid sequence comprises one or more of the following:
(a) valine at position 44;
(b) valine at position 64;
(c) glycine at position 103;
(d) arginine at position 104;
(e) proline at position 111;
(f) glycine at position 117;
   or a nucleotide sequence which is the complement of any aforesaid sequence.

The position numbers are stated with reference to SEQ ID NO: 13.

Preferably the nucleic acid encodes a polypeptide that enables sarcinaxanthin production as defined above (i.e. at the levels as defined above). More preferably the nucleic acid molecule comprises a nucleotide sequence as set forth in SEQ ID NO: 12 or a part of variant thereof as defined above, or a complement thereof.

The present invention further provides a nucleic acid molecule comprising (or consisting of) a nucleotide sequence encoding a protein which contributes to (or more particularly which is a subunit of a protein having) C₅₀ carotenoid γ-cyclase activity and which has an amino acid sequence as set forth in all or part of SEQ ID NO: 15 or an amino acid sequence which is at least 90% identical to SEQ ID NO: 15, wherein said amino acid sequence comprises one or more of the following:
(a) a glycine residue at position 100;
(b) a glycine residue at position 103;
(c) a proline residue at position 107;
   or a nucleotide sequence which is the complement of any aforesaid sequence.

The position numbers are stated with reference to SEQ ID NO: 15.

Preferably the nucleic acid molecule encodes a polypeptide that enables sarcinaxanthin production as defined above, e.g. at the levels defined above. More preferably the nucleic acid molecule comprises a nucleotide sequence as set forth in SEQ ID NO: 14 or a part of variant thereof as defined above, or a complement thereof.

Additionally, the present invention also provides a nucleic acid molecule comprising (or consisting of) a nucleotide sequence encoding all or part of a protein having an amino acid sequence as set forth in SEQ ID NO: 34 or an amino acid sequence which is at least 90% identical to SEQ ID NO: 34 and wherein said nucleotide sequence encodes a sarcinaxanthin glycosylase enzyme, which activity results in the production of both sarcinaxanthin mono- and diglucosides, when expressed in a host cell, or a nucleic acid molecule which comprises a nucleotide sequence which is the complement of any aforesaid sequence.

A nucleic acid molecule as defined in this aspect of the invention may comprise or consist of:
(i) a nucleotide sequence as set forth in SEQ ID NO: 33;
(ii) a nucleotide sequence which is degenerate with the sequence of SEQ ID NO: 33;
(iii) a nucleotide sequence which has at least 90% sequence identity to SEQ ID NO: 33;
(iv) a nucleotide sequence which is a part of the nucleotide sequence of SEQ ID NO: 33 or of a nucleotide sequence which is degenerate therewith; or
(v) a nucleotide sequence which is complementary to any of (i) to (iv) above.

Alternatively or additionally the present invention also provides a nucleic acid molecule comprising (or consisting of) a nucleotide sequence encoding a protein having sarcinaxanthin glycosylase activity and an amino acid sequence as set forth in all or part of SEQ ID NO: 34 or an amino acid sequence which is at least 90% identical to SEQ ID NO: 34, wherein said amino acid sequence comprises one or more of the following:
(a) histidine at position 62;
(b) serine at position 109;
(c) arginine at position 129;
(d) alanine at position 138;
(e) arginine at position 248;
(f) proline at position 251;
   or a nucleotide sequence which is the complement of any aforesaid sequence.

The position numbers are stated with reference to SEQ ID NO. 34.

Preferably the nucleic acid encodes a sarcinaxanthin glycosylase which enables sarcinaxanthin mono- or diglucoside production, as defined elsewhere herein. More preferably the nucleic acid molecule comprises a nucleotide sequence as set forth in SEQ ID NO: 33 or a part of variant thereof as defined above, or a complement thereof.

Hence, in one embodiment a sarcinaxanthin glycosylase or a nucleic acid encoding a sarcinaxanthin glycosylase as described herein may be used for the production of a sarcinaxanthin mono- or diglucoside. For instance, a nucleic acid encoding a sarcinaxanthin glycosylase may be introduced into a host cell capable of producing sarcinaxanthin to produce sarcinaxanthin mono- or diglucoside.

Additionally, the present invention also provides a nucleic acid molecule comprising (or consisting of) a nucleotide sequence encoding all or part of a protein having an amino acid sequence as set forth in SEQ ID NO: 36 or an amino acid sequence which is at least 90% identical to SEQ ID NO: 36 and wherein said nucleotide sequence encodes a protein of the sarcinaxanthin biosynthetic gene cluster, or a nucleic acid molecule which comprises a nucleotide sequence which is the complement of any aforesaid sequence.

A nucleic acid molecule as defined in this aspect of the invention may comprise or consist of:
(i) a nucleotide sequence as set forth in SEQ ID NO: 35;
(ii) a nucleotide sequence which is degenerate with the sequence of SEQ ID NO: 35;
(iii) a nucleotide sequence which has at least 90% sequence identity to SEQ ID NO: 35;
(iv) a nucleotide sequence which is a part of the nucleotide sequence of SEQ ID NO: 35 or of a nucleotide sequence which is degenerate therewith; or
(v) a nucleotide sequence which is complementary to any of (i) to (iv) above.

Alternatively or additionally the present invention also provides a nucleic acid molecule comprising (or consisting of) a nucleotide sequence encoding a protein of the sarcinaxanthin biosynthetic gene cluster and an amino acid sequence as set forth in all or part of SEQ ID NO: 36 or an amino acid sequence which is at least 90% identical to SEQ ID NO: 36, wherein said amino acid sequence comprises one or more of the following:
(a) valine at position 3;
(b) leucine at position 7;
(c) glutamine at position 22;
(d) glutamine at position 29;
(e) aspartic acid at position 33;
(f) methionine at position 34;
(g) threonine at position 41;
(h) threonine at position 50;
(i) serine at position 68;
(j) arginine at position 161;
(k) tyrosine acid at position 163;
(l) isoleucine at position 190;
(m) arginine acid at position 197;
(n) glutamic acid at position 199;
   or a nucleotide sequence which is the complement of any aforesaid sequence.

The position numbers are stated with reference to SEQ ID NO. 36.

Preferably the nucleic acid molecule comprises a nucleotide sequence as set forth in SEQ ID NO: 35 or a part of variant thereof as defined above, or a complement thereof.

The invention also extends to proteins or polypeptides encoded by the above-defined nucleic acids and use of the above-defined nucleic acids in the methods of the invention described elsewhere herein.

In general the term “gene” includes the ORF which encodes the protein, together with any regulatory sequences such as promoters, whereas the term “ORF” refers only to the part of the gene which is responsible for encoding the protein.

As referred to herein “functionally equivalent variants” or “functional equivalents” retain the activity of the entity to which they are related (or from which they are derived), e.g. encode or represent a protein with substantially the same properties, e.g. enzymatic or enzymatic subunit activity, and preferably retain the activity at substantially the same level as the parent entity. The properties or activities can be tested for using standard techniques that are known in the art. As used herein the term “substantially” can be taken to mean at least 90% and preferably at least 95, 96, 97, 98 or 99% of the activity of the parent entity.

A “part” of the nucleic acid molecule may contain at least 50%, more particularly at least 60, 70, 75, 80, 85, 90 or 95% of the nucleotides of the molecule. Thus by way of representative example it may be at least 180, or at least 200 bases in length, or at least 250, 280, 300, 500, 600, 700, 800, 900, 1000, 1500, 2000, 3000, 4000, 5000, 6000 or 7000 bases. In the context of a nucleic acid molecule representing the entire gene cluster, the fragment lengths will be longer. However, where molecules representing individual genes are concerned, representative part lengths will be lower. As mentioned above, a number of genes and ORFs have been identified within SEQ ID NO: 1, 26 and 37 and parts or fragments which comprise such genes or ORFs represent preferred “parts” or fragments of SEQ ID NO: 1, 26 and 37. However, also encompassed are parts or fragments of the SEQ ID NOs representing the individual genes or ORFs.

Nucleotide or amino acid sequence identity may be assessed by any convenient method. However, for determining the degree of sequence identity between sequences, computer programs that make multiple alignments of sequences are useful, for instance Clustal W (Thompson, J. D et al., 1994). Programs that compare and align pairs of sequences, like ALIGN (Myers, E. and Miller, W. 1988), FASTA (Pearson, W. R. and Lipman, D. J. 1988 and Pearson, W. R. 1990) and gapped BLAST (Altschul, S. F., et al., 1997) are also useful for this purpose. Furthermore, the Dali server at the European Bioinformatics institute offers structure-based alignments of protein sequences (Holm, 1993; Holm, 1995; Holm, 1998).

For example, nucleotide sequence identity may be determined using the BestFit program of the Genetics Computer Group (GCG) Version 10 Software package from the University of Wisconsin. The program uses the local homology algorithm of Smith and Waterman with the default values: Gap creation penalty=50, Gap extension penalty=3, Average match=10,000, Average mismatch=−9.000.

Thus for example, depending on the context, nucleotide sequence identity may be at least 70%, 75%, 85%, 87%, 90%, 91%, 92%, 93%, 94%, 95%, 96%, 97%, 98% or 99% to any nucleotide sequence (i.e. a nucleotide sequence of any SEQ ID NO.) stated herein (i.e. within the constraints and confines stated herein). Nucleotide sequences meeting the % sequence identity criteria defined herein may be regarded as “substantially identical” sequences or as functionally equivalent or variant sequences.

Programs for determining amino acid sequence identity are mentioned above, for example amino acid sequence identity or similarity may be determined using the BestFit program of the Genetics Computer Group (GCG) Version 10 Software package from the University of Wisconsin. The program uses the local homology algorithm of Smith and Waterman with the default values: Gap creation penalty −8, Gap extension penalty=2, Average match=2.912, Average mismatch=−2.003.

Thus for example, depending on the context, amino acid sequence identity may be at least 70%, 75%, 85%, 87%, 90%, 91%, 92%, 93%, 94%, 95%, 96%, 97%, 98% or 99% to any amino acid sequence (i.e. to an amino acid sequence of any SEQ ID NO.) stated herein (i.e. within the constraints and confines stated herein). Amino acid sequences meeting the % sequence identity criteria defined herein may be regarded as “substantially identical” sequences or as functionally equivalent or variant sequences.

The polypeptide/protein of the invention may be an isolated, purified or synthesized polypeptide. As noted above, the term “polypeptide” is used herein interchangeably with the term “protein” and includes any amino acid sequence of two or more amino acids, i.e. both short peptides and longer lengths are included.

A “part” of any protein or amino acid sequence as defined herein may contain at least 50%, more particularly at least 60, 70, 75, 80, 85, 90 or 95% of the amino acid residues of the molecule or sequence. A part may comprise at least 20 contiguous amino acids, preferably at least 30, 40, 50, 60, 70, 80, 90, 100, 110, 120, 150, 160, 170, 180, 190, 200, 210, 220, 240, 250, 260, 270 or 280 contiguous amino acids.

As noted above in relation to “functionally equivalent variants” or “functional equivalents”, a part of a nucleic acid or protein molecule, or of a nucleotide or amino acid sequence, as referred to herein advantageously retains the activity of the entity to which it is related (or from which it is derived), e.g. encodes or represents a protein with substantially the same properties, e.g. enzymatic or enzymatic subunit activity, and preferably retains the activity at substantially the same level as the parent entity. The part may thus correspond to, or comprise, an active site or functional part of the protein.

The nucleotide sequences described herein provide important tools and information which can be utilised in a number of ways to manipulate sarcinaxanthin biosynthesis, particularly to produce high levels of sarcinaxanthin through the heterologous expression of the biosynthetic machinery in host cells. By sarcinaxanthin biosynthetic machinery is meant a group of proteins (e.g. encoded by a gene cluster) that comprises one or more proteins that are involved in the sarcinaxanthin biosynthetic pathway, which is functional in sarcinaxanthin synthesis, but which is not necessarily restricted only to the presence of sarcinaxanthin biosynthetic enzymes or enzymatic domains, e.g. genes/proteins isolated from M. luteus strains. Thus, as noted above, certain proteins may replaced with functionally-equivalent counterparts from (e.g. derived from) other sources, that is proteins which catalyse the same conversions, or which exhibit the same or equivalent activity.

Although the nucleic acids used in the methods of the invention may correspond to native genes/ORFs or may encode native proteins, as noted above the respective nucleotide and/or amino acid sequences may be modified. The modification may take place by modifying one or more nucleotide sequences so as to cause the modification of one or more encoded proteins. This may result in alteration of enzyme activity e.g. improved enzymatic activity and consequently may enhance yields of sarcinaxanthin or derivatives thereof. Alternatively, such a modification may be desirable to facilitate the operation of the method, for example construction of an expression vector etc, or otherwise in the manipulation of the nucleic acids, or it may result in improved expression etc, or enable expression in a different host etc. Thus, by way of example, nucleic acid molecules of the invention may be utilised to manipulate or facilitate the biosynthetic process, for example by extending the host range or increasing yield or production efficiency etc.

As described in more detail below, recombinant expression of a nucleic acid molecule according to the invention may involve the introduction of one or more nucleic acid molecules into a host cell (e.g. a heterologous host cell) and the culturing (or growth) of that host cell under conditions which allow the nucleic acid molecule to be expressed and sarcinaxanthin or a derivative thereof to be produced (i.e. conditions which allow the expression product(s) of the nucleic acid molecule to synthesise sarcinaxanthin). In such a recombinant expression system, the nucleic acid molecule may be subject to modification before being introduced into the host cell and expressed.

In certain embodiments a host may be used which already contains some of the genes required to make precursors in the sarcinaxanthin pathway, e.g. a lycopene-producing host cell. In such a host, modification of the genes which are already present in the host may take place in situ. In other words, in a lycopene-producing host for example, the endogenous genes already present for lycopene production may be altered, for example to increase lycopene production, e.g. by gene replacement, the introduction of new regulatory sequences or mutagenesis.

In the methods of the invention, the nucleic acid molecules may be any of the nucleic acid molecules of the invention as defined herein, namely nucleic acid molecules containing nucleotide sequences corresponding to, or derived from, the Otnes7 genes. However, whilst in certain aspects this is preferred, particularly in the context of the biosynthetic pathway from lycopene, due to the greater efficiency of these genes in sarcinaxanthin production, this is not mandatory and nucleic acid molecules from or based on other sources may be used. Thus, for example, as noted above lycopene is a common intermediate in a number of pathways, and may be synthesised by a number of different organisms. Nucleic acid molecules based on known gene sequences for proteins involved in lycopene production may be used. In terms of the sarcinaxanthin biosynthesis pathway beyond lycopene, nucleic acid molecules corresponding to, or derived from, any M. luteus genes may be used, e.g. corresponding to, or derived from, the crtE2 and/or crtYgYh genes of any strain of M. luteus may be used, including in particular strain NCTC2665.

Thus, in one embodiment the method of the present invention may comprise introducing into a lycopene-producing host cell and expressing:

(a) a nucleic acid molecule encoding a protein capable of catalysing the conversion of lycopene to flavuxanthin, or alternatively put a protein having lycopene elongase activity;

(b) a nucleic acid molecule encoding a C₅₀ carotenoid γ-cyclase subunit and comprising:
(i) a nucleotide sequence as set forth in all or part of SEQ ID NO: 2 or SEQ ID NO: 12, or which is degenerate therewith, or which has at least 70% sequence identity to SEQ ID NO: 2 or 12;
(ii) a nucleotide sequence which hybridizes to SEQ ID NO: 2 or 12 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 3 or 13 or an amino acid sequence which is at least 70% identical to SEQ ID NO: 3 or 13; and

(c) a nucleic acid molecule encoding a C₅₀ carotenoid γ-cyclase subunit and comprising:
(i) a nucleotide sequence as set forth in all or part of SEQ ID NO: 4 or 14, or which is degenerate therewith, or which has at least 70% sequence identity to SEQ ID NO: 4 or 14;
(ii) a nucleotide sequence which hybridizes to SEQ ID NO: 4 or 14 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 5 or 15 or an amino acid sequence which is at least 70% identical to SEQ ID NO: 5 or 15.

Thus, in the context of (a), (b) and (c) above, the method may involve the introduction of a single nucleic acid molecule encoding, e.g. crtE2, crtYh and crtYg (or proteins with the equivalent functional activity) from either the NCTC2665 or preferably the Otnes7 strains of M. luteus. Alternatively, two or more separate molecules may be introduced. Preferably the nucleic acid molecules used in the invention comprise any combination of the nucleic acid molecules as defined herein.

In one embodiment of the invention the method results in the production of sarcinaxanthin to a level of at least 150 μg/g of cell dry weight (CDW). Preferably, sarcinaxanthin is produced to a level of at least 300, 500, 750, 1000, 2000, 2500 μg/g CDW.

In a further embodiment the method of the invention results in a host cell, wherein at least 91% of the total carotenoids produced is sarcinaxanthin. Preferably, at least 92, 93, 94, 95, 96, 97, 98 or 99% of the total carotenoids produced is sarcinaxanthin.

A lycopene-producing host cell may be any cell that is capable of producing lycopene, preferably in significant amounts, e.g. at least 0.5, 0.6, 0.7, 0.8, 1.0 or 1.5 mg/g CDW. In other words, a lycopene-producing cell comprises the biosynthetic machinery necessary to produce lycopene, wherein said machinery may be present naturally or endogenously as part of the host cell genome or said machinery or parts thereof may be introduced into said host cell to enable said cell to produce lycopene. For example, the sarcinaxanthin biosynthetic machinery comprises genes encoding enzymes capable of producing lycopene, i.e. crtE, crtB and crtI. Thus, the method of the invention includes the introduction and expression of one or more nucleic acid molecules comprising a nucleotide sequence as set forth in all or part of any one of SEQ ID NOs: 18, 20, 22, 27, 29, 31 and 33, or which are degenerate therewith, or which are at least 70% identical to SEQ ID NOs: 18, 20, 22, 27, 29, 31 or 33, or which are otherwise related to SEQ ID NOs 18, 20, 22, 27, 29, 31 or 33 by analogy to the definitions given above in relation to SEQ ID NOs. 2, 4, 12 or 14 or their corresponding amino acid sequences. Alternatively, the endogenous lycopene biosynthetic machinery of the host cell may be modified so as to enhance lycopene production in said host.

As mentioned above, the lycopene biosynthetic pathway has been extensively described and more than one pathway is known to exist, e.g. the MEP pathway described above and in the carotenoid biosynthetic pathway in plants and cyanobacteria (see e.g. Cunningham et al., 1994). Hence, any combination of genes encoding enzymes that result in the production of lycopene in the host cell, whether endogenous or heterologously expressed is encompassed by the present invention.

In a preferred aspect, the lycopene producing host cell comprises genes encoding the CrtE, CrtI and CrtB proteins from Pantoea ananatis or parts or functional equivalents thereof, wherein said genes are expressed. In other words, the host cell comprises genes encoding three enzymes for the biosynthesis of lycopene from isoprenyl pyrophosphate (IPP) and dimethylallyl pyrophosphate (DMAPP). Said genes may be integrated into the host genome or present in the form of a plasmid or equivalent thereof. Conveniently, the lycopene producing host cell may comprise the plasmid pAC-LYC (Cunningham and Gantt, 2007).

As discussed above, enzymes capable of catalysing the conversion of lycopene to flavuxanthin, i.e. lycopene elongases, are known in the art, e.g. crtEb from Corynebacterium glutamicum, and nucleic acid molecules encoding any enzymes with an equivalent functional activity may be used in the methods of the invention. In a preferred aspect of the present invention the nucleic acid molecule encoding a protein capable of catalysing the conversion of lycopene to flavuxanthin may be a nucleic acid molecule comprising:
(i) a nucleotide sequence as set forth in all or part of SEQ ID NO: 6, 7 or 10, or which is degenerate therewith, or which has at least 70% sequence identity to SEQ ID NO: 6, 7 or 10;
(ii) a nucleotide sequence which hybridizes to SEQ ID NO: 6, 7 or 10 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 8, 9 or 11 or an amino acid sequence which is at least 70% identical to SEQ ID NO: 8, 9 or 11.

More preferably, the nucleic molecule which encodes an enzymes capable of catalysing the conversion of lycopene to flavuxanthin is a nucleic acid molecule of the invention as defined above.

A sarcinaxanthin derivative can be defined as any modification of the sarcinaxanthin molecule, e.g. the addition of further chemical groups, wherein said groups may or may not alter the functional properties of sarcinaxanthin. Such a derivative may for example be a glycosylated derivative, for example which may carry one or two glycosyl groups. As described in the examples, the sarcinaxanthin biosynthetic gene cluster encodes a sarcinaxanthin glycosylase enzyme, which activity results in the production of both sarcinaxanthin mono- and diglucosides. Thus, in a preferred embodiment of the invention the method comprises the introduction of a further nucleic acid molecule into said host cell, wherein said nucleic acid molecule encodes an enzyme capable of glycosylating sarcinxanthin. More preferably, said nucleic acid molecule encodes crtX from M. luteus or a functional equivalent thereof. Most preferably, the nucleic acid comprises: (i) a nucleotide sequence as set forth in all or part of SEQ ID NO: 16 or 33, or which is degenerate therewith, or a nucleotide sequence with at least 70% sequence identity to SEQ ID NO: 16 or 33;
(ii) a nucleotide sequence which hybridizes to SEQ ID NO: 16 or 33 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 17 or 34 or which comprises an amino acid sequence which is at least 70% identical to SEQ ID NO: 17 or 34.

Further preferably, the nucleic acid molecule comprises a nucleotide sequence encoding a protein having sarcinaxanthin glycosylase activity and an amino acid sequence as set forth in all or part of SEQ ID NO: 34 or an amino acid sequence which is at least 90% identical to SEQ ID NO: 34, wherein said amino acid sequence comprises one or more of the following:
(a) histidine at position 62;
(b) serine at position 109;
(c) arginine at position 129;
(d) alanine at position 138;
(e) arginine at position 248;
(f) proline at position 251;
   or a nucleotide sequence which is the complement of any aforesaid sequence.

The position numbers are stated with reference to SEQ ID NO. 34.

Preferably the nucleic acid encodes a sarcinaxanthin glycosylase which enables sarcinaxanthin mono- or diglucoside production, as defined elsewhere herein. More preferably the nucleic acid molecule comprises a nucleotide sequence as set forth in SEQ ID NO: 33 or a part of variant thereof as defined above, or a complement thereof.

Alternatively, sarcinaxanthin produced according to the invention may be glycosylated by glycosylase enzymes or other glycosylation mechanisms which are present in the host cell. Further, the sarcinaxanthin produced according to the invention may be glycosylated in vitro according to procedures well known in the art.

Also included as part of the invention are cells into which a nucleic acid molecule has been introduced, namely a heterologous host cell, for example in accordance with any of the methods as hereinbefore defined, or cells into which a nucleic acid molecule of the invention has been introduced.

To enable heterologous expression of a nucleic acid molecule(s) of the invention, the invention also provides a vector, for example a cloning or preferably an expression vector, comprising a nucleic acid molecule of the invention. Said vector may then be introduced into the host cell for expression of said nucleic acid molecule and therefore production of sarcinaxanthin.

Generally speaking to perform the methods of the invention an appropriate expression vector may include appropriate control sequences such as for example translational (e.g. start and stop codons, ribosomal binding sites) and transcriptional control elements (e.g. promoter-operator regions, termination stop sequences) linked in matching reading frame with the nucleic acid molecules required for performance of the method of the invention as described herein. Appropriate vectors may include plasmids and viruses (including, e.g. bacteriophage). Preferred vectors include bacterial expression vectors, e.g. pBAD-vectors, pET-vectors and pTRC-vectors. The nucleic acid molecule may conveniently be fused with DNA encoding an additional polypeptide, e.g. glutathione-S-transferase, to produce a fusion protein on expression.

A range of vectors are possible and any convenient or desired vector may be used. A vast range of vectors and expression systems are known in the art and described in the literature and any of these may be used or modified for use according to the present invention. Vectors may be used which are based on the broad-host-range RK2 replicon, into which an appropriate strong promoter may be introduced. For example WO 98/08958 describes RK2-based plasmid vectors into which the Pm/xylS promoter system from a TOL plasmid has been introduced. Such vectors represent preferred vectors which may be used according to the present invention. Alternatively, any vector containing the Pm promoter may be used, whether in plasmid or any other form, e.g. a vector for chromosomal integration, for example a transposon-based vector.

Other vectors or expression systems which may be used include for example those based on the pET, pBT, pMyr, pSos, pTRG or pGen expression systems. Promoters that may be useful in the expression of the proteins according to the invention include, but are not limited to, the lac promoter, T7, Ptac, PtrcT7 RNA polymerase promoter (P₇φ10), λP_{L} and P_{BAD}. The vectors may, as noted above, be in autonomously replicating form, typically plasmids, or may be designed for chromosomal integration. This may depend on the host organism used, for example in the case of host cells of Bacillus sp. chromosomal integration systems are used industrially, but are less widely used in other prokaryotes. Generally speaking for chromosomal integration, transposon delivery vectors for suicide vectors may be used to achieve homologous recombination. In bacteria, plasmids are generally most widely used for protein production.

Thus viewed from a further aspect, the present invention provides a vector, preferably an expression vector, comprising a nucleic acid molecule as defined above.

Other aspects of the invention include methods for preparing recombinant nucleic acid molecules according to the invention, comprising inserting nucleotide sequences encoding the polypeptides of the invention into vector nucleic acid.

Any suitable expression system may be used in the host cell and will be dependent on the nature of said cells. The vector may comprise any number of other genetic elements, e.g. for selection, integration of the nucleic acids into the host genome, regulation of the expression of the nucleic acid molecules etc. The regulatory elements may be derived from various sources that are well known in the art. Such regulatory elements may result in the constitutive expression of said nucleic acid molecules or may be inducible. As noted above, in a preferred embodiment of the invention, the nucleic acid molecules used in the methods discussed above are under the control of the Pm/xylS promoter system.

The Pm/xylS promoter system has been shown to function in a wide range of gram negative bacterial species, and has been found useful for over-expression of recombinant proteins (Mermod et al., 1986; Ramos et al., 1988; Blatny et al. 1997a). The uninduced expression level from Pm is low, and the use of different effector compounds at various concentrations can be used to regulate the level of induced expression (Winther-Larsen et al., 2000a). Many of the inducers are low-cost compounds that enter the cell by passive diffusion.

The Pm/xylS expression system has been used in the construction of broad-host range expression vectors based on the RK2 minimal replicon (Blatny et al., 1997b; Blatny et al., 1997a; and WO98/08958). One of these vectors, pJB658, has proven useful for tightly regulated recombinant gene expression in several gram-negative species (Blatny et al., 1997b; Blatny et al, 1997a; Brautaset et al., 2000; Winther-Larsen et al., 2000b). For example, this vector has been used for recombinant expression of a host-toxic single-chain antibody fragment (scFv), hGM-CSF and hIFN-2ab (Sletta et al., 2004; Sletta et al., 2007).

Introduction of a vector (e.g. a plasmid) or more than one vector comprising the nucleic acid molecules as defined herein into the appropriate host cell can be performed using routine methods in the art. This may ultimately result in the integration of the nucleic acid molecule(s) into the genome of the host cell or said vector may exist as an autonomic replicating unit within the host cell.

The resultant modified host cell will therefore contain a sarcinaxanthin biosynthetic gene cluster, which encodes a sarcinaxanthin enzyme system. The sarcinaxanthin biosynthetic machinery will be expressed and thus synthesise sarcinaxanthin molecules.

A preferred embodiment of the present invention involves the isolation of genes from a native organism which synthesises sarcinaxanthin, e.g. M. luteus NCTC2665 or Otnes7, or from an organism which synthesizes a sarcinaxanthin precursor such as lycopene of flavuxanthin, optionally modifying said genes, and the introduction of said genes into a host cell, i.e. an organism other than M. luteus, for expression and production of sarcinaxanthin and derivatives thereof.

Generally speaking, the nucleic acid molecule will be expressed in a host cell under conditions in which the biosynthetic machinery may be expressed. The host cell may be grown or cultured under conditions which allow the nucleic acid molecules and biosynthetic machinery to be expressed, and sarcinaxanthin or a derivative thereof to be synthesised.

Thus, the nucleic acid molecule may be expressed in any desired host cell, but preferably it will be expressed in a cell or microorganism other than that from which it was (or from which it may be) derived and in which the molecule is natively present.

The methods of the invention for producing sarcinaxanthin or a derivative thereof may further comprise the step of recovering (e.g. isolating or purifying) sarcinaxanthin, e.g. from the culture medium in which the host cell was grown or from the host cell. This can be isolated or purified from the cell culture medium into which it has been transported or secreted if appropriate, or otherwise from the host cell in which it has been produced. Thus, for example, the cells of the producing organism may be harvested, e.g. by centrifugation, and sarcinaxanthin or a derivative thereof may be extracted following cell lysis, for example with organic solvent(s) (e.g., methanol and acetone in a ratio of 7:3). The sarcinaxanthin or derivatives thereof may be recovered from such an extract, for example by precipitation or evaporation. Further purification of a crude product obtained in this way may include e.g. chromatography, e.g. HPLC.

As noted above, in one aspect the invention provides a host cell containing one or more nucleic acid molecules as defined above, wherein said molecule(s) has been introduced into said host cell.

By way of representative example, the crtE2YgYh regions of the M. luteus strain Otnes7, may be amplified from genomic DNA and inserted into an expression vector, e.g. pJBphOx. Said expression vector may then be introduced into a host cell, e.g. E. coli XL1 Blue containing the pAC-LYC plasmid (described above). The host cell may then be cultivated such that the proteins encoded by the pAC-LYC and expression vectors are expressed thereby resulting in the production of sarcinaxanthin.

Alternatively, a host cell (e.g. microorganism) which endogenously contains one or more nucleic acid molecules required for synthesis of a sarcinaxanthin precursor, e.g. lycopene or flavuxanthin, may be modified by introduction of one or more nucleic acid molecules which encode proteins which are capable of catalysing the conversion of lycopene to flavuxanthin to sarcinaxanthin, for example by simple introduction of the nucleic acid molecule, or by e.g. gene replacement, for example to replace the gene encoding the flavuxanthin-converting activity in the host cell. Thus for example, C. glutamicum cells mays be modified to replace or supplement the crtYeYf genes with a nucleic acid molecule encoding a γ-cyclase activity, including any such molecule as defined herein.

The host cell for use in the methods of the invention may be any desired cell or organism, prokaryotic or eukaryotic, but generally it will be a microorganism particularly a bacterium. More particularly, the host cell will be an Escherichia coli cell or a Corynebacterium glutamicum cell. Other representative host cells include both Gram negative and Gram positive bacteria. Suitable bacteria include Escherichia sp., Salmonella, Klebsiella, Proteus, Yersinia, Azotobacter sp., Pseudomonas sp., Xanthomonas sp., Agrobacterium sp., Alcaligenes sp., Bordatella sp., Haemophilus influenzae, Methylophilus methylotrophus, Rhizobium sp., Thiobacillus sp. and Clavibacter sp. In a particularly preferred embodiment, expression of the desired gene product occurs in E. coli. Eukaryotic host cells may include yeast cells or mammalian cell lines.

Preferably the host cells do not endogenously contain all of the nucleic acid molecules required for the synthesis of sarcinaxanthin or a derivative thereof, but may preferably comprise nucleic acid molecules encoding proteins required for the synthesis of sarcinaxanthin precursors, e.g. lycopene, nonaflavuxanthin or flavuxanthin. A suitable example is the E. coli XL1 Blue strain comprising the pAC-LYC plasmid (Cunningham and Gantt, 2007).

The novel isolated strain referred to above, from which the gene cluster was also sequenced (isolate Otnes7), as deposited under deposit number DSM 23579 at the DSMZ, may be used for the production of sarcinaxanthin, but is not a preferred host cell of the methods of the invention. However, this strain represents an important aspect of the present invention and a preferred source of the nucleic acid molecules for use in the methods of the invention, particularly nucleic acid molecules encoding proteins crtE2, crtYg and crtYh. The endogenous nucleic acid molecules of the sarcinaxanthin biosynthetic gene cluster of this strain may be modified as described herein (i.e. directly or indirectly) to identify nucleic acid molecules that encode proteins with further improved enzyme activity/substrate to product conversion efficiency. Alternatively, the Otnes 7 strain may be mutagenized and screened to identify isolates with improved sarcinaxanthin activity. Genes from the sarcinaxanthin gene cluster may then be isolated and used in the methods of the invention.

A further aspect of the present invention is thus a strain of Micrococcus luteus as deposited under number DSM 23579 at the DSMZ, or a mutant or modified strain thereof which produces sarcinaxanthin or a derivative thereof.

The sarcinaxanthin produced by the methods of the invention may be further modified for example by glycosylation or other derivatisation, in order to exhibit or improve activity, e.g. antioxidant activity. Methods for glycosylating carotenoids are generally known in the art; the glycosylation may be effected intracellularly by providing the appropriate glycosylation enzymes or may be effected in vitro using chemical synthetic means.

Mutations can be made to the native sequences using conventional techniques. The substrates for mutation can be an entire cluster of genes or only one or two of them; the substrate for mutation may also be portions of one or more of these genes. Techniques for mutation are well known in the art and described in the literature. Such techniques include preparing synthetic oligonucleotides including the mutation(s) and inserting the mutated sequence into the gene using restriction endonuclease digestion. Alternatively, the mutations can be effected using a mismatched primer (generally 15-30 nucleotides in length) which hybridizes to the native nucleotide sequence, at a temperature below the melting temperature of the mismatched duplex. The primer can be made specific by keeping primer length and base composition within relatively narrow limits and by keeping the mutant base centrally located. Primer extension is effected using DNA polymerase, the product cloned and clones containing the mutated DNA, derived by segregation of the primer extended strand, selected. The technique is also applicable for generating multiple point mutations. PCR mutagenesis will also find use for effecting the desired mutations.

The vectors used to perform the various operations described above may be chosen to contain control sequences operably linked to the resulting coding sequences in a manner that expression of the coding sequences may be effected in the host. However, simple cloning vectors may be used as well.

The invention will now be described in more detail in the following non-limiting Examples with reference to the drawings in which:

FIG. 1: Proposed biosynthetic pathway for the individual steps in the formation of sarcinaxanthin and its glucosides from lycopene. CrtEBI: GGPP synthase, phytoene synthase, phytoene desaturase; CrtE2: lycopene elongase; CrtYg+CrtYf: C₅₀ carotenoid γ-cyclase; CrtX: C₅₀ carotenoid glycosyl transferase.

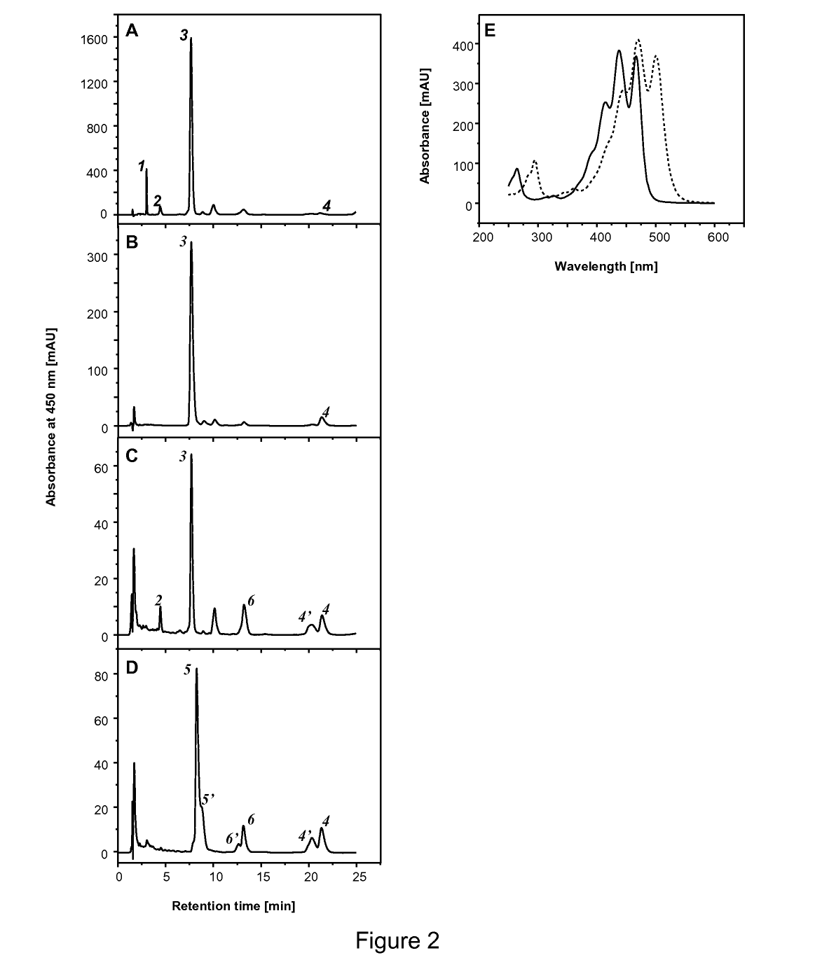

FIG. 2: HPLC elution profile of carotenoids extracted from M. luteus strain Otnes7 (A), lycopene-producing E. coli XL1 Blue pAC-LYC transformed with pCRT-E2YgYh-O7 (B), pCRT-E2YgYhX-O7 (C) and pCRT-E2-O7 (D). Peak 1, sarcinaxanthin diglucoside; peak 2, sarcinaxanthin monoglucoside; peak 3, sarcinaxanthin; peak 4, lycopene; peak 5, flavuxanthin; peak 6, nonaflavuxanthin; Peak 4′ 5′ and 6′ are the cis isomers of 4, 5 and 6 respectively. Absorption spectra of carotenoids from peaks 1, 2 and 3 (solid line) and peaks 4, 5 and 6 (scattered line) are depicted in graph (E).

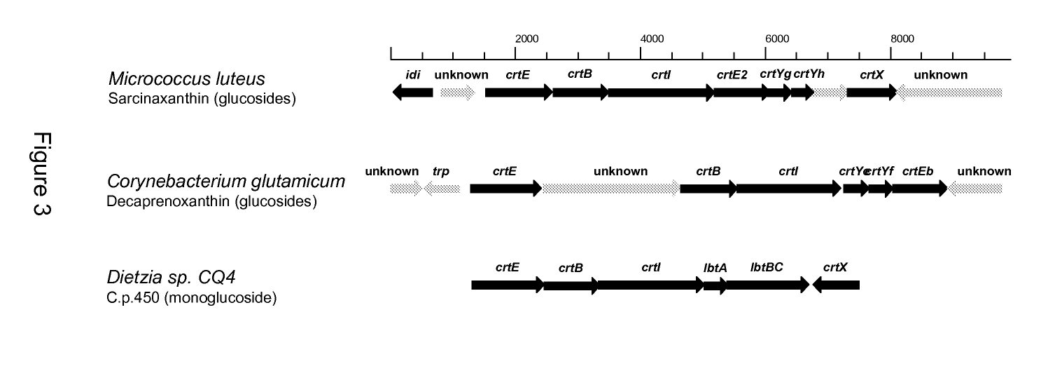

FIG. 3: Carotenoid biosynthesis gene clusters from M. luteus, C. glutamicum and Dietzia sp. leading to C₅₀ carotenoids sarcinaxanthin, decaprenoxanthin, C.p. 450 and its glycosylated derivatives, respectively. Genes indicated in grey are suggested not to be involved in carotenoid biosynthesis.

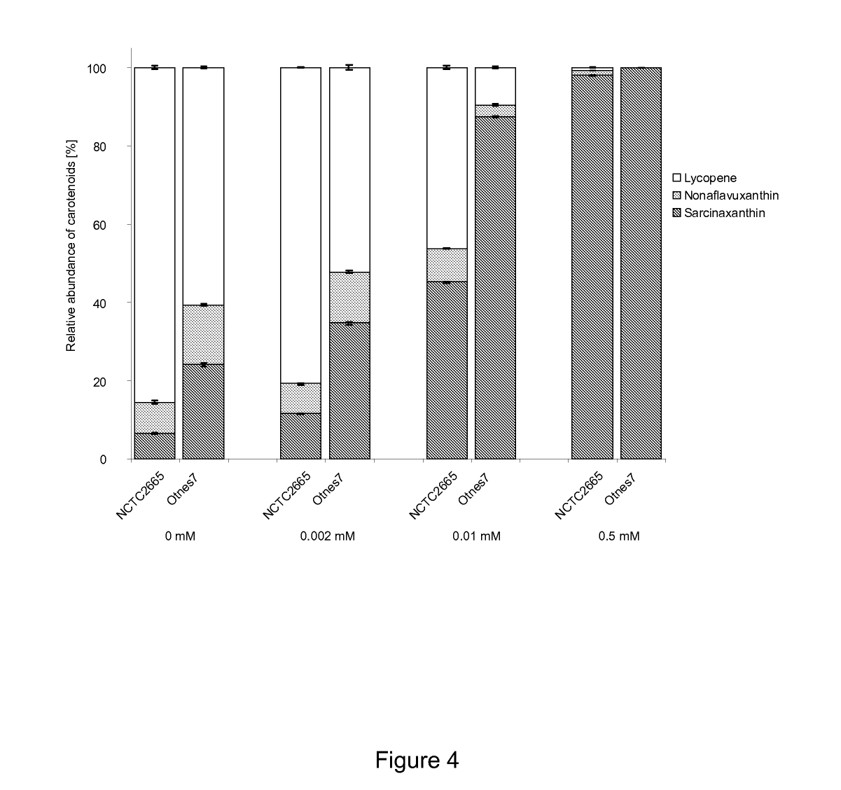

FIG. 4: The relative carotenoid abundance in extracts from E. coli pAC-LYC overexpressing crtE2YgYh genes from M. luteus strain Otnes7 and strain NCTC2665 cultivated in the presence of 0, 0.002, 0.01 and 0.5 mM m-toluate. The fraction of sarcinaxanthin, lycopene and intermediates are indicated by dark grey, white and light grey columns, respectively. Samples were analyzed after 48h of cultivation. The extracted total carotenoid was similar in the presented samples and 100% carotenoid abundance corresponds to [x]±[y] mg/g CDW total carotenoid.

### EXAMPLES

### Example 1

### Materials and Methods

Bacteria, Plasmids, Standard DNA Manipulations, and Growth Media

Bacterial strains and plasmids used in this work are listed in Table 2. Bacteria were cultivated in Luria-Bertani (LB) broth (Sambrook, Fritsch et al. 1989), and recombinant E. coli cultures were supplemented with ampicillin (100 μg/ml) and chloramphenicol (30 μg/ml). M. luteus and C. glutamicum strains were grown at 30° C. and 225 rpm agitation, while E. coli strains were generally grown at 37° C. and 225 rpm agitation. For heterologous production of carotenoids, 250 ml cultures of recombinant E. coli strains were grown at 28° C. with 180 rpm agitation in 500 ml Erlenmeyer shake flasks for 24 h in the presence of 0.5 mM of the Pm inducer m-toluate, unless otherwise stated. Standard DNA manipulations were performed according to Sambrook et al., (1989) and isolation of total DNA from M. luteus strains was performed as described elsewhere (Tripathi and Rawal 1998).

Vector Constructions

pCRT-EBIE2YgYh-2665 and pCRT-EBI-2665:

The complete crtEBIE2YgYh gene cluster of M. luteus NCTC2665 was PCR amplified from genomic DNA by using the primer pair crtE-F (5′-TTTTTCATATGGGTGAAGCGAGGACGGG-3′) and crtYh-R (5′-TTTTTGCGGCCGCTCAGCGATCGTCCGGGTGGGG-3′). The crtEBI region of M. luteus NCTC2665 was PCR amplified from genomic DNA by using the primer pair crtE-F (see above) and crtI-R (5′-TTTTTGCGGCCGCTCATGTGCCGCTCCCCCCGG). The resulting PCR products, crtEBIE2YgYh (5283 bp) and crtEBI (3693 bp), were end digested with NdeI and NotI (indicated in bold in primer sequences) and ligated into the corresponding sites of pJBphOx (Sletta et al., 2004), yielding plasmids pCRT-EBIE2YgYh-2665 and pCRT-EBI-2665, respectively.

pCRT-E2YgYh-2665 and pCRT-E2YgYh-O7:

The crtE2YhYg regions of M. luteus strains NCTC2665 and Otnes7 were PCR amplified from genomic DNA using primers crtE2-F (5′-TTTTTCATATGATCCGCACCCTCTTCTG-3′) and crtYh-R (see above). The obtained 1615 by PCR products were blunt end ligated into pGEM-Teasy vector system (Promega, Madison, Wisc.), and the resulting plasmids were digested with NdeI and NotI and the 1597 by inserts were ligated into the corresponding sites of pJBphOx, yielding plasmids pCRT-E2YgYh-2665 and pCRT-E2YgYh-O7, respectively.

pCRT-E2YgYhX-O7:

The crtE2YgYhX region of M. luteus strain Otnes7 was PCR amplified from genomic DNA using primers crtE2-F (see above) and crtYX-R: (5′-TTTTTCCTAGGAGATGGCCGCGAACATCCTG). The obtained PCR product was end digested with NdeI and BlnI (indicated in bold in the primer) and the corresponding 3085 by fragment ligated into the corresponding sites of pJBphOx, resulting in pCRT E2YgYhX-O7.

pCRT-E2Yg-O7 and pCRT-E2Yg-2665:

The crtE2Yg coding regions of M. luteus strains NCTC2665 and Otnes7 were PCR amplified from chromosomal DNA using primers crtE2-F (see above) and crtYg-R (5′-TTTTTGCGGCCGCTCACCGGCTCCCCCGGTCGGTC-3′). The obtained PCR products were end digested with NdeI and NotI (indicated in bold in primer sequence) and resulting 1247 by fragments ligated into the corresponding sites of pJBphOx, resulting in pCRT-E2Yg-O7 and pCRT-E2Yg-2665, respectively.

pCRT-E2-O7 and pCRT-E2-2665:

The crtE2 genes of M. luteus strains NCTC2665 and Otnes7 were PCR amplified from chromosomal DNA using primers crtE2-F (see above) and crtE2-R (5′-TTTTTGCGGCCGCTCATGCCGCCGCCCCCCGGG-3′). The resulting PCR products were end digested with NdeI and NotI (indicated in bold in the primer sequence) and the corresponding 890 by fragments ligated into likewise treated pJB658phOx, resulting in pCRT-E2-O7 and pCRT-E2-2665, respectively.

pCRT-YgYh-O7 and pCRT-YgYh-2665:

The YgYh regions of M. luteus strains NCTC2665 and Otnes7 were PCR amplified from genomic DNA by using primers crtYg-F (5′-TTTTTCATATGATCTACCTGCTGGCCCT-3′) and crtYh-R (see above). The resulting 734 by PCR products were end digested with digested with NdeI and NotI (indicated in bold in the primer sequences) and resulting 716 by fragments were ligated into the corresponding sites of pJB658phOx, resulting in pCRT-YgYh-O7 and pCRT-YgYh-2665, respectively.

pCRT-E2YeYf-Hybrid:

According to the gene sequences of crtE2 in M. luteus Otnes7 and crtYeYf in C. glutamicum MJ233-MV10, four primers crtE2-F (5′-TGACCAACGACCGGTAGCGGAG-3′) and crtE2-i-R (5′-CCCATCCACTAAACTTAAACATCATGCCGCCGCCCCCCGG-3′), crtYe-i-F (5′-TGTTTAAGTTTAGTGGATGGGTTGATCCCTATCATCGATATTTCAC-3′) and crtYf-R (5′-TTTTGCGGCCGCTTTTCCATCATGACTACGGCTTTTC) were used. Primers crtE2-i-R and crtYe-i-F contain homologous extensions of 21 bp (italic) at the 5′ ends as linker sequences in order to allow cross over PCR. Primer pair crtE2-F and crtE2-i-R was used to amplify a 1227 by fragment containing the crtE2 gene from genomic M. luteus DNA and primer pair crtYe-i-F and crtYf-R was used to amplify a 885 by crtYeYf containing fragment from genomic C. glutamicum DNA. The resulting PCR fragments were used as template for PCR with primer pair crtE2-F and crtYe-R to amplify a 2090 by hybrid DNA fragment containing crtE2 from M. luteus and crtYeYf from C. glutamicum connected by the 21-bp linker sequence. The resulting hybrid fragment was end digested with AgeI and NotI (indicated in bold in primer sequence) and the obtained 2070 by DNA fragment ligated into the corresponding sites of pJB658phOx, resulting in vector pCRT-E2YeYf-Hybrid.

pCRT-YeYfEb-MJ:

The crtYeYfEb genes from C. glutamicum strain MJ-233C-MV10 were PCR amplified from genomic DNA using primers crtYe-F1 (5′-TGGCTATCTCTAGAAAGGCCTACCCCTTAGGCTTTATGCAACAGAAACAATAATAATGGAG TCATGAACATATGATCCCTATCATCGATATTTCAC-3′) and crtYf-R (5′-TTTTGCGGCCGCCTGATCGGATAAAAGCAGAGTTATATC-3′). The resulting PCR product was digested with XbaI and NotI (indicated in bold in primer sequence) and the resulting 1789 by DNA fragment was ligated into the corresponding sites of pJBphOx, yielding pCRT-YeYfEb-MJ.

All the constructed vectors were verified by DNA sequencing and transformed by electroporation (Dower, Miller et al. 1988) into E. coli strain XL1-blue and the lycopene producing E. coli strain XL1-blue (pAC-LYC), respectively (Cunningham, Sun et al. 1994).

Extraction of Carotenoids from Bacterial Cell Cultures

To extract carotenoids from M. luteus strains, cells were harvested, washed with deionized H₂O, treated with lysozyme (20 mg/ml) and lipase (Fluka Chemicals, Germany) according to (Kaiser, Surmann et al. 2007) and the pigments were extracted with a mixture of methanol and acetone (7:3). For recombinant E. coli strains, 50 ml aliquots of the cell cultures were centrifuged at 10,000×g for 3 min and the pellets were washed with deionized H₂O, the cells were then frozen and thawed to facilitate extraction. Finally the pigments were extracted with 4 ml methanol/acetone at 55° C. for 15 min with thorough vortex every 5 min. When necessary, up to three extraction cycles were performed to remove all colours from the cell pellet. When selective extraction for xanthophylls was desired, pure methanol was used. 0.05% butylhydroxytoluene (BHT) was added to the organic solvent to contribute to the stabilization of carotenoids. Samples for preparative HPLC were in addition partitioned into 50% diethyl ether in petroleum ether. The collected upper phase was evaporated to dryness and dissolved in methanol.

Quantification of Carotenoids in Cell Extracts

Carotenoids were quantified on the basis of the area in the chromatographic analysis and by using a standard curve made by known concentrations of a trans-beta-apo-8′-carotenal and lycopene standard (Fluka). The correct concentrations of the standard was determined spectrophotometrically (Harker and Bramley 1999) by using the extinction coefficients E 1 cm 1% of 3450 for lycopene and 2590 for apo-carotenal. Standards were filtered through a syringe 0.2 μm polypropylene filter (Pall Gelman) and stored in amber glass vessels at −80° C. under N₂ atmosphere if not analyzed immediately.

LC-MS Analyses

LC-MS analyses were performed on an Agilent Ion Trap SL mass spectrometer equipped with an Agilent 1100 series HPLC system. The HPLC system was equipped with a diode array detector (DAD) which recorded UV/VIS spectra in the range from 200-650 nm. Two HPLC protocols were used for the analysis in this work. A high throughput protocol for a fast quantitative determination of known carotenoids was used as follows; the carotenoids were eluted isocratically in MeOH for 5 min. A Zorbax rapid resolution SB RP C₁₈ column with dimension 2.1*30 mm was used for the analyses. Column flow was kept at 0.4 mL/min and 10 μL extract was injected for each run. For detailed qualitative carotenoid separation a Zorbax SB RP C₁₈ with dimension 2.1*150 mm was used. The carotenoids were eluted isocratically in MeOH/Acetonitrile (7:3) for 25 minutes. The column flow was 250 μl/min and 10 or 20 μL sample was injected depending on the concentration.

For determination of the molecular masses of carotenoids, mass spectrometry (MS) was performed under the following conditions. Analytes were ionized using a chemical ionization source with settings 325° C. dry temperature, 350° C. vaporizer temperature, 50 psi nebulizer pressure and 5.0 L/min dry gas. The MS was operated in scan mode. For carotenoid identification, preparative HPLC was performed on an Agilent preparative HPLC 1100 series system equipped with two preparative HPLC pumps, a preparative autosampler and a preparative fraction collector. Mobile phases were methanol in channel 1 and acetonitrile in channel 2. Samples of 2 mL were injected at a flow rate of 20 mL/min to a Zorbax RP C18 2.1*250 mm preparative LC column. On-line MS analysis was performed by splitting the flow 1:200 after the column using an Agilent LC flow splitter and a make-up flow of 1 mL methanol/min was used to carry the analytes to the MS with less than 15 sec delay. The diode array detector was used to trigger fraction collection.

Carotenoid structure determination by NMR

All NMR spectra were recorded on a Bruker Avance 600 MHz instrument, fitted with a TCI cryoprobe using CDCl₃ as solvent with TMS as internal reference. ¹H and ¹³C signals were unambiguously assigned by the aid of ip-COSY, HSQC, HMBC, NOESY and HSQC-TOCSY experiments.

### Example 2

### Analysis of Carotenoids Produced by M. luteus Strains NCTC2665 and Otnes7

We initially characterised the major carotenoids synthesized by M. luteus, and the recently genome sequenced M. luteus NCTC2665 was chosen as one model strain. Cell extracts from shake flask cultures were analyzed by LC-MS and one major peak (peak 3) (FIG. 2A) was identical to that of the sarcinaxanthin standard purified and structurally identified by NMR earlier M. luteus (Stafsnes et al., 2010). In addition, two minor peaks, peak 1 and peak 2, were identified with the same absorption spectra as that of sarcinaxanthin (FIG. 2A). The retention time of peak 2 was equal to sarcinaxanthin monoglucoside identified by NMR earlier (Stafsnes et al., 2010), while peak 1 was more polar and therefore here predicted to represent sarcinaxanthin diglucoside (Table 3).

Several M. luteus strains from the sea surface microlayer of the mid-part of the Norwegian coast has previously been isolated and characterized for their sarcinaxanthin production capacities (Stafsnes et al., 2010). One selected isolate, designated Otnes7, forms bright yellow colonies on LB agar plates and with higher colour intensity than that of strain NCTC2665. Otnes7 was here classified as a M. luteus strain by 16S-rRNA sequence analysis (93% identical to NCTC2665), and this strain was included as a second model strain. Qualitative analysis of extracts confirmed that strain Otnes7 produces the same carotenoids as NCTC2665, while the total carotenoid level (190 μg/g CDW) of Otnes7 cells was higher than that of NCTC2665 cells (145 μg/g CDW). The latter result was in agreement with the different colour intensities of the respective bacterial colonies, and this was further investigated.

### Example 3

### Cloning and Genetic Characterisation of the M. luteus NCTC2665 crtEIBE2YgYh Sarcinaxanthin Biosynthetic Gene Cluster

The genome sequence of M. luteus strain NCTC2665 was deposited in the databases (Accession number: NC_{—}012803). In silico screening of the DNA sequence data resulted in identification of a putative carotenoid biosynthesis gene cluster consisting of eight open reading frames, or1007, or1009-or1014 and ORF1. The genetic organization of crt genes in M. luteus displayed certain similarities to the previously published biosynthetic gene clusters for the C₅₀ carotenoids C.p. 450 and decaprenoxanthin in Dietzia sp. (Tao, Yao et al. 2007) and C. glutamicum (Krubasik, Kobayashi et al. 2001), respectively (FIG. 3).

### Example 4

### Expression of the crtEIBE2YgYh Genes Resulted in Production of Non-Glycosylated Sarcinaxanthin in E. coli

To experimentally test if the identified M. luteus gene cluster encoded an active sarcinaxanthin biosynthetic pathway, the crtEBIE2YgYh region from NCTC2665 was cloned in frame and under transcriptional control of the positively regulated Pm promoter in plasmid pJBphOx (Sletta et al., 2004). This expression vector has many favourable properties useful for regulated expression of genes and pathways under relevant levels in gram-negative bacteria (for review, see Brautaset et al., 2009). The resulting plasmid pCRT-EBIE2YgYh-2665 was transformed into the non-carotenogenic E. coli host strain XL1-blue, and the recombinant strain was analysed for carotenoid production under induced conditions (0.5 mM m-toluic acid). LC-MS analysis of cell extracts revealed a small peak at identical retention time, absorption spectrum, and relative molecular mass as sarcinaxanthin identified in M. luteus strains. The recombinant E. coli strain produced small amounts of sarcinaxanthin (10 to 15 μg/g CDW), which was not present in plasmid free cells, confirming that the identified gene cluster encodes a sarcinaxanthin biosynthetic pathway from FFP.

### Example 5

### Sarcinaxanthin Production Levels can be Increased Up to 150-Fold by Expressing Otnes7 crtE2YgYh Genes and in a Lycopene Producing E. coli Host

To overcome the poor sarcinaxanthin production levels obtained (above) a recombinant strain E. coli XL1 Blue (pCRT-EBI-2665) was established, expressing three enzymes catalyzing the conversion of FFP into lycopene (FIG. 1). Analysis of this recombinant strain under induced conditions confirmed that it produced lycopene. However, the production levels (8-12 μg/g CDW) remained low; analogous with the sarcinaxanthin levels obtained with E. coli XL1 Blue (pCRT-EBIE2YgYh-2665) (see above). Therefore, E. coli XL1-blue was transformed with plasmid pAC-LYC (Cunningham and Gantt 2007) harbouring the Pantoea ananatis crtEBI genes encoding three enzymes for biosynthesis of lycopene from IPP (isoprenyl pyrophosphate) and DMAPP (dimethylallyl pyrophosphate). LC-MS analysis confirmed that the resulting strain XL1-blue (pAC-LYC) accumulated significant amounts of lycopene (1.8 mg/g CDW) as sole carotenoid. Therefore, all further carotenoid production experiments were performed by using XL1-blue (pAC-LYC) as a host.

XL1-blue (pAC-LYC) (pCRT-E2YgYh-2665), and LC-MS analysis of cell extracts revealed a total carotenoid accumulation of 2.3 mg/g CDW and about 90% of the total carotenoid produced was identified as sarcinaxanthin. These data demonstrated that the M. luteus NCTC2665 crtE2YgYh gene products can effectively convert lycopene into sarcinaxanthin in a lycopene producing cell under these conditions. We also established and analysed the strain XL1-blue (pAC-LYC) (pCRT-EBIE2YgYh-2665) and the results were similar as for XL1-blue (pAC-LYC) (pCRT-E2YgYh-2665) strain. The latter result implies that the M. luteus crtEBI gene products are not efficient for lycopene production in E. coli, and whether this is due to poor expression levels or low catalytic activities in this host, remained unknown.

An analogous strain XL1 Blue (pAC-LYC) (pCRT-E2YgYh-O7) was established, and the total carotenoid production level (2.5 mg/g CDW) of the resulting recombinant strain was slightly higher than that of analogous strain XL1 Blue (pAC-LYC) (pCRT-E2YgYh-2665). 97% of the total carotenoid produced by XL1 Blue (pAC-LYC) (pCRT-E2YgYh-O7) was sarcinaxanthin indicating efficient conversion of the lycopene. It should also be noted that the sarcinaxanthin production levels obtained in this heterologous host was above 10-fold higher than those obtained by the two M. luteus strains under such conditions (see above). To further compare the efficiency of using Otnes7 versus NCTC2665 derived biosynthetic genes, production analyses were performed with different Pm inducer concentrations (FIG. 4). The results demonstrated that strain XL1-blue (pAC-LYC) (pCRT-E2YgYh-O7) produced sarcinaxanthin to significantly higher levels than strain XL1-blue (pAC-LYC) (pCRT-E2YgYh-2665) under all conditions tested, thus confirming that Otnes7 genes are preferable for efficient sarcinaxanthin production in an E. coli host. This result was in agreement with the higher sarcinaxanthin production levels of Otnes7 compared to NCTC2665 (see above). DNA sequence analysis of the cloned Otnes7 crtE2YgYh fragment revealed in total 24 nucleotide substitutions compared to the corresponding NCTC2665 DNA sequence, resulting in three amino acid substitutions in CrtE2, six in CrtYg, and two substitutions plus one insertion in CrtYh. It is proposed that one or more of these sequence variations positively affects the expression level or the catalytic properties of the respective proteins.

### Example 6

### Expression of crtE2 and crtE2Yg Resulted in Accumulation of C₄₅ Nonaflavuxanthin and C₅₀ Flavuxanthin

To elucidate the detailed biosynthetic steps for the conversion of lycopene to sarcinaxanthin, recombinant strain XL1 Blue (pAC-LYC) (pCRT-E2-2665) was established and analysed for carotenoid production. Two different carotenoids were accumulated in the cells in addition to lycopene (FIG. 2D); all three compounds shared identical UV/Vis profiles. No sarcinaxanthin was detected. The minor carotenoid had a molecular mass of 620 Da, indicating a C₄₅ carotenoid and the major carotenoid had a molecular mass of 704 Da indicating a C₅₀ carotenoid. The major carotenoid was separated by preparative HPLC and analyzed by NMR. Inspection of ¹H, ¹³C and HSQC spectra revealed chemical shifts in agreement with reported data for the acyclic C₅₀ carotenoid flavuxanthin (Krubasik, Takaichi et al. 2001). The minor carotenoid was identified as nonaflavuxanthin on the basis of the UV/Vis profile and the mass (Table 3). These results verified that the M. luteus crtE2 gene encodes a lycopene elongase catalyzing the sequential elongation of the C₄₀ carotenoid lycopene via the C₄₅ carotenoid nonaflavuxanthin to the C₅₀ carotenoid flavuxanthin. A similar analysis by using the analogous strain XL1 Blue (pAC-LYC) (pCRT-E2-O7) gave the same conclusion. Interestingly, the relative conversion of lycopene was substantially higher in the latter strain (79% vs. 23%), which was in agreement with the generally higher sarcinaxanthin production level obtained when expressing Otnes7 genes (see FIG. 4).

We then constructed and analysed recombinant strains XL1 Blue (pAC-LYC) (pCRT-E2Yg-O7) and XL1 Blue (pAC-LYC) (pCRT-E2Yg-2665). The carotenoids produced by both strains were flavuxanthin, nonaflavuxanthin and lycopene and their relative abundance was similar to strains XL1 Blue (pAC-LYC) (pCRT-E2-O7) and XL1 Blue (pAC-LYC) (pCRT-E2-2665), respectively. Taken together our data thus imply that the CrtYg and CrtYh polypeptides must function together as an active C₅₀ carotenoid cyclase catalyzing cyclization of flavuxanthin to sarcinaxanthin in vivo. To our knowledge, this γ-type of carotenoid cyclase enzyme has not previously been described. To unravel if this cyclase can also catalyse cyclization of lycopene, we established and analysed recombinant strains XL1 Blue (pAC-LYC) (pCRT-YgYh-O7) and XL1 Blue (pAC-LYC) (pCRT-YgYh-2665). HPLC analysis showed that both strains accumulated lycopene, confirming that the crtYgYh gene products can not use lycopene as a substrate in vivo.

### Example 7

### The crtX Gene Product Encodes an Active Glycosyl Transferase that can be Used to Produce Monoglycosylated Sarcinaxanthin in E. coli Host

Immediately downstream of crtYh there is a an ORF encoding a hypothetical protein, followed by or1007 which encodes a putative polypeptide sharing 43% primary sequence identity to the putative glycosyl transferase protein CrtX (FIG. 3) from Dietzia sp., suggested to be involved in the glycosylation of C.p. 450 (Tao, Yao et al. 2007). To our knowledge, no analogous gene has been found in the C. glutamicum genome sequence and still this bacterium can synthesize glycosylated decaprenoxanthin (Krubasik, Takaichi et al. 2001). The or1007 gene was herein named crtX, and to unravel its biological function we constructed and analysed recombinant strain XL1 Blue (PAC-LYC) (pCRT-E2YgYhX-O7). The resulting HPLC profile (FIG. 2C) revealed sarcinaxanthin as the major carotenoid (peak 3), but an additional more polar carotenoid was eluted earlier (peak 2) which had an identical retention time and absorption spectrum to that of sarcinaxanthin monoglucoside from M. luteus Otnes 7 (FIGS. 2C and E). Another minor peak was observed with the same retention time as that of sarcinaxanthin diglucoside; however, the detected amount was too low for a confident analysis of the mass and absorption spectrum. Interestingly, about 10% of the total produced sarcinaxanthin was glycosylated both in M. luteus and when produced heterologously in E. coli. These results confirmed that crtX encodes an active glycosyl transferase that is necessary for the glycosylation of sarcinaxanthin under the conditions tested.

Based on all accumulated data we could deduce the complete biosynthetic pathway of sarcinaxanthin and its glucosides from FFP and via lycopene in M. luteus (FIG. 1), and this represents to our knowledge the first experimentally confirmed biosynthetic pathway of a γ-cyclic C₅₀ carotenoid.

TABLE 2
Bacterial strains and plasmids used for heterologous production
of sarcinaxanthin and other C₅₀ carotenoids

| Strain/Plasmid | Relevant characteristics | Reference source |
|---|---|---|
| Strain | | |
| E. coli DH5α | General cloning host | Gibco-BRL |
| E. coli XL1-blue | General cloning host | Stratagene |
| M. luteus NCTC2665 | | National collection |
| | | of Type Cultures |
| M. luteus Otnes7 | Marine wild type isolate | This work |
| C. glutamicum MJ-233C- | Tn31831 mutant of C. glutamicum MJ-233C; | (Kurusu, Kainuma |
| MV10 | contains wild type crt gene cluster | et al. 1990; Vertes, |
| | | Asai et al. 1994; |
| | | Krubasik, Takaichi |
| | | et al. 2001) |
| Plasmid | | |
| pGEM-T | Amp^{r}; Standard cloning vector | Promega, |
| | | Madison, USA |
| pJBphOx | Amp^{r}, pJB658 derivative | (Sletta, Nedal et al. |
| | | 2004) |
| pAC-LYC | Cm^{r}, lycopene producing plasmid containing | (Cunningham, |
| | crtEIB from P. ananatis, p15A ori | Chamovitz et al. |
| | | 1993) |
| pGEM-TcrtE2YgYh-O7 | Amp^{r}, pGEM-T with crtE2YgYh fragment | This work |
| | from strain Otnes7 | |
| pGEM-TcrtE2YgYh-2665 | Amp^{r}, pGEM-T with crtE2YgYh fragment | This work |
| | from strain NCTC2665 | |
| pCRT-EBIE2YgYh-2665 | Amp^{r}, pJBphOx with phOx fragment | This work |
| | substituted with crtEBIE2YgYh fragment from | |
| | strain Otnes7 | |
| pCRT-EBI-2665 | Amp^{r}, pJBphOx with phOx fragment | This work |
| | substituted with crtEBI fragment from strain | |
| | NCTC 2665 | |
| pCRT-E2YgYh-O7 | Amp^{r}, pJBphOx with phOx fragment | This work |
| | substituted with crtE2YgYh fragment from | |
| | strain Otnes7 | |
| pCRT-E2YgYh-2665 | Amp^{r}, pJBphOx with phOx fragment | This work |
| | substituted with crtE2YgYh fragment from | |
| | strain NCTC 2665 | |
| pCRT-E2Yg-O7 | Amp^{r}, pJBphOx with phOx fragment | This work |
| | substituted with crtE2Yg fragment from strain | |
| | Otnes7 | |
| pCRT-E2Yg-2665 | Amp^{r}, pJBphOx with phOx fragment | This work |
| | substituted with crtE2Yg fragment from strain | |
| | NCTC2665 | |
| pCRT-E2-O7 | Amp^{r}, pJBphOx with phOx fragment | This work |
| | substituted with crtE2 fragment from strain | |
| | Otnes7 | |
| pCRT-E2-2665 | Amp^{r}, pJBphOx with phOx fragment | This work |
| | substituted with crtE2 fragment from strain | |
| | NCTC2665 | |
| pCRT-YgYh-O7 | Amp^{r}, pJBphOx with phOx fragment | This work |
| | substituted with crtYgYh fragment from strain | |
| | Otnes7 | |
| | Amp^{r}, pJBphOx with phOx fragment | |
| pCRT-YgYh-2665 | substituted with crtYgYh fragment from strain | This work |
| | NCTC2665 | |
| pCRT-E2YgYhX-O7 | Amp^{r}, pJBphOx with phOx fragment | This work |
| | substituted with crtE2YgYhX fragment from | |
| | strain Otnes7 | |
| pCRT-E2-O7-YeYf-MJ | Amp^{r}, pJBphOx with phOx fragment | This work |
| | substituted with crtE2 fragment from strain | |
| | Otnes7 and YeYf from C. glutamicum MJ- | |
| | 233C-MV10 | |
| pCRT-YeYfEb-MJ | Amp^{r}, pJBphOx with phOx fragment | This work |
| | substituted with crtYeYfEb fragment from | |
| | C. glutamicum MJ-233C-MV10 | |
| pCRT-E2Yg-2665-Yf-MJ | Amp^{r}, pJBphOx with phOx fragment | This work |
| | substituted with a crtE2Yg fragment from | |
| | strain Otnes7 and crtYf fragment from | |
| | C. glutamicum | |

TABLE 3
Characteristics of carotenoids extracted from M. luteus strain Otnes7
and carotenoids produced heterologously with E. coli strains^{a}.

| | | | | Relative | Retention |
|---|---|---|---|---|---|
| Carotenoid | λₘₐₓ (nm) in the HPLC | | | molecular | time |
| (trivial name) | eluent | | | mass (m/z) | Rₜ (min) |
| Sarcinaxanthin | 414 | 438 | 467 | 1028 | 3.0 |
| diglucoside | | | | | |
| Sarcinaxanthin | 414 | 438 | 467 | 886 | 4.5 |
| monoglucoside | | | | | |
| Sarcinaxanthin | 414 | 438 | 467 | 704 | 7.7 |
| Flavuxanthin | 445 | 470 | 501 | 704 | 8.2 |
| Nonaflavuxanthin | 445 | 470 | 501 | 620 | 13.2 |
| Lycopene | 445 | 470 | 501 | 536 | 21.3 |
| Decaprenoxanthin | 414 | 438 | 467 | 704 | 10.1 |

^{a}Carotenoids dissolved in MeOH and separated by HPLC using the system including the Zorbax C18 150*30 column

Sequences:

```
SEQ ID NO: 1 - M.luteus NCTC2665 sarcinaxanthin gene cluster
      1 gcggagtcct cgtccgcctc ggcgtcgtcg ctgtccgcgg ccccggccga ctacgaggcc
    61 ggcacgtgct tcaccgcccc gctcggcgcg cgtgacctgt cctccttcga gaccaccgac
  121 tgcgagggcg cccacaccgc ggagtacctg tgggccgtgc cggccgtggc cgagggtgag
  181 gaggccgacc ccgccgccgc ccagacctgc accgcccagg cccagcgcct gagcgaggag
  241 aaggaggacc agctgaacgg ggccgtcctg acctcctccg agctgggcaa ctacggcacc
  301 gacgagaagc actgcgtcgt gtacggggtc tccggtgagt gggagggtca gatcgtggac
  361 ccggagatca ccctggagac ggcgtccgcc gacgcctgat cccgccggcg gccccgtgcg
  421 tcgtgagatc gcgccgcccg ggaccgccgc ggatggacgc gggaccggcg cggcccgtag
  481 tgtcttctgc gtccagaagt tagacggtcg aacaggtgcg gcggtcggtg ccgcgtcgtg
  541 tccgccaccg aggaggcgcc atgggtgaag cgaggacggg cggcgaggcc gcgctctccg
  601 gggtgaccgc cgagctggac gccgcgctcc gacacgccgc ggcccaggcg cccggatccg
  661 ccgccttcgc cgagctgctc gactcgctcc acgtccatgt gggcgccggc aagctcatcc
  721 gcccccgtct cgtcgagctc ggctggcgcc tggcgaccgc cgacccggtc cctccgtccg
  781 gccgcgctgc cgtcgaccga ctcggggccg ccttcgaact gctgcacacc gcgctgctcg
  841 tccacgacga cgtcatcgat cgggacgtgc tgcggcgcgg ccagcccgcc gtgcacgcct
  901 ccgcccggca ccgcctcgag gcccgcgggg tgcccgccgc ggacgccgcc cacgccgggg
  961 tcgccgtcgc cctcatcgcg ggggacgtcc tgctcaccca ggcgttccgg ctcgccgcca
1021 cctgtgccgc cgacaccgcc cgggccgccg aggccgccgc cgtcgtcttc gacgccgccg
1081 ccgtgactgc ggccggcgag ctcgaggacg tgctcctggg gctgtcccgc cacaccggtg
1141 aggagcccga tcccgaccgc atcctcgcca tgcaacggct caagacggcg cactacacgg
1201 tcggcgcgcc cctgcgcgcc ggcgccctcc tggccggggc ggatcccgac ctcgcccggg
1261 cgatgggcga ggccggcgcc gacctcggcg ccgcctacca ggtgatcgac gacgtcctcg
1321 gcgtgttcgg cgatcccggg gagaccggca agtccgccga cggcgacctg cgcgagggca
1381 aggccaccgt gctcaccgcc cacggccgcc gcatccccgc cgtccgcgcc ctgctcgacg
1441 cgggcccggc cacccccgcg gacatcgagg ccgcccgccg cgccctcgag gcggccggtg
1501 cccgggagca cgccctcgac gtcgccgccg agctcaccgt ccgcgcccgc gagcgcatcg
1561 cggccctgcc cctggacgag acggtccggg cggagttcgc cgacgcctgc cacgccgtgc
1621 tgacccggag gtcctgagat ggccgcgccc accccgagcc ctgccgcgct gtacacgcgg
1681 acggcccaca ccgcagcggc ccaggtgatc cgccgctact ccacgtcctt ctcctgggcc
1741 tgccgcaccc tgccccggca ggcacgccag gacgtggcca cgatctacgc catggtccgc
1801 gtcgccgacg aggtggtcga cggcgtcgcg gtggccgccg ggctcgacga ggccggggtc
1861 cgcgccgccc tggacgacta cgagcgggcg tgtgaggccg cgatggcgtc gggcttcgcc
1921 accgacccgg tcctgcacgc cttcgccgac gtggcccgtc gccacggcat caccccggag
1981 ctgacccgtc ccttcttcgc ctccatgcgc gcggacctgg ggatccgcga gcacggcgcc
2041 gagtccctgg acgcctacat ccacggctcg gccgaggtgg tggggctgat gtgcctgcag
2101 gtcttcctct ccctccccgg cacgcgggcc cggaccccgg gccagcggca ggagctgcgc
2161 gcgcaggcct cccggctggg ggcggcgttc cagaaggtca acttcctcag ggacctggcc
2221 gcggaccacc acgagctggg ccgcacctac ctgcccggtg ccgcaccggg cgtgctcacc
2281 gaggcccgca aggccgagct cgtggccgag gtccgcgccg acctcgacgc cgccctgccc
2341 ggcatccgtg tcctggaccc cggggccggg cgcgccgtgg ccctggcgca cggactgttc
2401 gcggccctgg tggaccggat cgaggcgacc ccggcggccg agctggccca ccgccgtgtc
2461 cgggtgccgg accatcagaa ggcccggatc gccgcccgcg tcctggcacg gggccgccgg
2521 ggaggccgcc gatgagcgcc cgggacaccg ctctcggccc gcgcaccgtg gtggtgggcg
2581 gcggtttcgc cggactggcc acggcgggcc tgttggcccg cgacgggcac cgggtgacgc
2641 tgctggagcg cggcgccgtc ctgggcggcc gtgccggacg ctggtccgag gcggggttca
2701 ccttcgatac cgggccctcc tggtacctga tgcccgaggt gatcgaccgc tggttccgcc
2761 tcatggggac ctccgccgcc gaacggctgg acctgcgccg tctggacccc ggctaccggg
2821 tgtacttcga ggggcacctc cacgagcccc ccgtggacgt gcgcaccggc cacgcggaga
2881 cgctgttcga gtccctcgag cccggcgccg ggcgccggct gcgggcctac ctcgactccg
2941 cgtcccggat ctacgggctc gccaaggagc acttcctcta cacggacttc cgccggccgg
3001 ccgccctggc ccacccggac gtcctgcgcg ccctgccggc cctcgggccc cagctgctgg
3061 ggggcctgcg ctcccacgtc gcggcccgct tccaggaccc ccggctgcgc cagatcctgg
3121 gctacccggc ggtcttcctc ggcacgtccc ccgaccgtgc ccccgccatg taccacctga
3181 tgtcccatct ggacctcgcc gacggcgtgc agtaccccct cggcgggttc gcggccctcg
3241 tggacgccat ggcggaggtc gtgcgcgagg ccggcgtgga gatccgcacc ggggtcgagg
3301 cgaccgccgt ggaggtcgcg gaccgtcccg cccccgccgg ccgcctcgga cgcctggccg
3361 cccgcctgcc caggccggga gcagcccgcg gggacgaggg ccgacgtcgc cgcccgggcc
3421 gggtgaccgg cgtcgcctgg cggtccgacg acggcgccgc gggacgcctc gacgccgatg
3481 tggtggtggc cgccgcggac ctgcaccacg tgcagacccg tctgctgcct cccggccggc
3541 gcgtcgcgga gtccacgtgg gaccggcgcg accccggccc ctccggcgtg ctcgtgtgcg
3601 tgggggtgcg cggatccctg ccccagctgg cccatcacac cctgctgttc acggcggact
3661 gggaggacaa cttcgggcgc atcgagcggg gggaggacct cgccgcggac acgtcgatct
3721 acgtctcgcg cacctccgcc acggacccgg gcgtggcccc ggagggcgac gagaacctct
3781 tcatcctcgt cccggccccc gccgagccgg ggtgggggcg cggcggcatc cgggtccgtg
3841 acggccaggg ctggcgggtg gaccgcgccg gggacgccca ggtggaggcc gtggcggacc
3901 gggccctcga tcagctggcc cgctgggccg ggatccccga cctggccgag cgcatcgtgg
3961 tgcggcgcac ctacgggccc ggtgacttcg ccgcggacgt gcacgcctgg cggggttcgc
4021 tgctgggccc cgggcacacg ctggcgcagt cggccatgtt ccgcccctcg gtgcgggacg
4081 cggacgtggc cggcctgatg tacgcgggct cctcggtgcg cccgggaatc ggggtgccca
4141 tgtgcctgat ctccgccgaa gtggtccggg acgaactgcg ccacgacgcg cgcagggccc
4201 ggcccgcggg ccccgggggg agcggcacat gatccgcacc ctcttctggg tgtcccggcc
4261 ggtcagctgg gtgaacacgg cctacccgtt cgccgccgcc gcgatcctga ccggggggct
4321 gcccgcgtgg ctggtggtcc tgggcgtcgt gttcttcctg gtgccctaca acctggccat
4381 gtacggcatc aatgacgtgt tcgacttcgc ctcggacctg cgcaaccccc gcaagggggg
4441 tgtggagggc tccgtgctgg gcgaccccgc ggtgcgccgc cgggtgctgg cgtggtcggt
4501 gctgctgccc gtgccgttcg tggccgtgct cgcgggctgg tccgccgtgc ggggcgagtg
4561 ggccgccgtg ctggtgctcg cggtgagcct gttcgcggtg gtggcgtact cctgggcggg
4621 gctgcggttc aaggagcggc ccttcctgga cgccgccacc tccgccaccc acttcgtctc
4681 ccccgcggtc tacggcctcg cgctggccgg ggcgaccccc acgcccgccc tggcggcgct
4741 gctgggggcg ttcttcctgt ggggcatggc ctcgcagatg ttcggggcgg tgcaggacgt
4801 ggtgccggac cgggaggggg gcctggcctc ggtggccacc gtgctgggcg ctcggcgcac
4861 cgtcctgctc gccgccggcc tgtacgcggc ggcgggcctg ctgctgctgg ccaccgaccc
4921 gccgggcccg ctcgcggcgc tgctggccgt gccctacgtg gtgaacaccc tgcgcttccg
4981 ccgcatcacg gacgccacct cgggcgcggc ccaccgcggc tggcagctgt tccttccgct
5041 gaactacgtg accggcttcc tcgtgaccct gctgctgatc gggtgggcgc tgacccgggg
5101 ggcggcggca tgatctacct gctggccctg ctgggtgtca tcggctgcat gctgctggtg
5161 gaccggcgct tcgagctgtt cctgtggcat cgcccgctcc cggcgctgct ggtgctggcc
5221 gccggggtgg cctacttctt cgcctgggac ctgtggggga tcgccgaagg cgtgttcctg
5281 caccggcagt cgccctacat gaccggggtg atgctcgccc cccagctgcc cctggaggag
5341 gggttcttcc tgctcttcct cagccagatc acgatggtgc tgttcaccgg ggcgctgcgc
5401 ctgctgcgcg gccggcgagg tgacgcccgt gccgcgacgg cggccgatcc gaccgaccgg
5461 gggagccggt gaccttcctc gacctcgtcc tcgtcttcgt gggcttcgcc ctggccgtgc
5521 tcgtgggcgc cgccctcgtc ggccgcgtgc ggggcgagca cctgcgggcc gtggcggcca
5581 ccctggtggc cctgtgggcc ctcacggcgg tcttcgacaa cgtgatgatc gccgcggggc
5641 tcttcgacta cggccatgag ctgctggtgg gtgcctacgt gggccaggcg cccgtggagg
5701 acttcgccta cccgctcggc tccgccctgc tgctgccggc gctctggctg ctgctgacga
5761 gccgtcgtgc cgatcggcgc ggccgtcggc cgggacgccg cccccacccg gacgatcgct
5821 gacatgctgc cgttgatccc cgcagacctg ctgcgcgcgc tcggcctgat cctcgtcccg
5881 gtcgcggcgg tgcacgccgg atggccgtcc gcggcggcga tgctgctcgt gttcggctcc
5941 cagtggctca cccgctggct cgccccgggc ggcgccctgg actgggccgc gcaggcggtc
6001 ctgctgctgg ccgggtggct gagcgtcatc ggcctctacc cgcgggtgcc gtggctggac
6061 ctgctcgtgc acgccgccgc ctccgccgtg gtcgcctgtc tgacggcact ggtggtgggg
6121 gcgtggctcc ggcgtcgggg gaccgaggcc gggcaggccg tggcgctgct cggcccgggc
6181 ctggccgggc tggggatcgc ggccgccgcc gtggccctgg gcgtggtgtg ggagctggcc
6241 gaatggtggg ggcacacggc ggtgaccccg gagatcggcg tgggctacac ggacaccatc
6301 ggcgacctcg ccgccgatct cgtcggcgcc ggggtcggcg ccgccctcgc cgtgtgccgg
6361 gggcgcaccc ggtgaccccg gcccgcccca cggtctccgt ggtcgtcccg gtgctcgacg
6421 acgccgagca cctgcgcgtg tgcctcgcgc tgctggccgc ccagagccgg ccggcgctgg
6481 aggtggtggt ggtggacaac ggctgcgtgg acgactcggc ggtgctcgcc cgcgccgccg
6541 gcgcgcgggt ggtgcgcgag ccgcgccgcg gggtcccggc cgcggcggcc gccggcctgg
6601 acgccgcggt cggggagctg ctggtgcgct gcgacgccga cacgcggatg cccgcggact
6661 ggctcgaacg gatcgtggcc cggttcgacg ccgaccccgg gctcgacgcc ctcaccgggc
6721 cggggacctt ccacgaccag cccggcctcc ggggacaggt gcgggcggcg ctctacaccg
6781 gcacgtaccg ctggggggcg ggcgccgcgg tggcggccac ccccgtctgg ggctccaact
6841 gcgccctgcg cgccgaggcg tggcaggctg tgcggacccg cgtccaccgc gaacgcgggg
6901 acgtgcacga tgacctggac ctgtccttcc agctggccct ggccggccgc cggatccggt
6961 tcgatccgga cctgcgggtg gaggtcgccg ggcgcatctt ccactccctg cgccagcggg
7021 tgcggcaggg ccggatggcg gtcaccaccc tgcaggtcaa ctgggcccga ctgtcccccg
7081 ggcggcgttg gctgcgccgg gcggcccggg cacacccccg gtcccgctgg gggcgtggcc
7141 ccgacggtca gtcccgggac tga
SEQ ID NO: 2 - M.luteus NCTC2665 crtYa nucleotide sequence
atgatctacctgctggccctgctgggtgtcatcggctgcatgctgctggtggaccggcgcttcgagctgttcctgtggcatcgcccgctc
ccggcgctgctggtgctggccgccggggtggcctacttcttcgcctgggacctgtgggggatcgccgaaggcgtgttcctgcaccggca
gtcgccctacatgaccggggtgatgctcgccccccagctgcccctggaggaggggttcttcctgctcttcctcagccagatcacgatgg
tgctgttcaccggggcgctgcgcctgctgcgcggccggcgaggtgacgcccgtgccgcgacggcggccgatccgaccgaccggg
ggagccggtga
SEQ ID NO: 3 - M.luteus NCTC2665 CrtYq polypeptide sequence
MIYLLALLGVIGCMLLVDRRFELFLWHRPLPALLVLAAGVAYFFAWDLWGIAEGVFLHRQSPYM
TGVMLAPQLPLEEGFFLLFLSQITMVLFTGALRLLRGRRGDARAATAADPTDRGSR
SEQ ID NO: 4 - M.luteus NCTC2665 crtYh nucleotide sequence
gtgaccttcctcgacctcgtcctcgtcttcgtgggcttcgccctggccgtgctcgtgggcgccgccctcgtcggccgcgtgcggggcgag
cacctgcgggccgtggcggccaccctggtggccctgtgggccctcacggcggtcttcgacaacgtgatgatcgccgcggggctcttc
gactacggccatgagctgctggtgggtgcctacgtgggccaggcgcccgtggaggacttcgcctacccgctcggctccgccctgctg
ctgccggcgctctggctgctgctgacgagccgtcgtgccgatcggcgcggccgtcggccgggacgccgcccccacccggacgatc
gctga
SEQ ID NO: 5 - M.luteus NCTC2665 CrtYh polypeptide sequence
VTFLDLVLVFVGFALAVLVGAALVGRVRGEHLRAVAATLVALWALTAVFDNVMIAAGLFDYGHE
LLVGAYVGQAPVEDFAYPLGSALLLPALWLLLTSRRADRRGRRPGRRPHPDDR
SEQ ID NO: 6 - M.luteus NCTC2665 crtE2 nucleotide sequence
atgatccgcaccctcttctgggtgtcccggccggtcagctgggtgaacacggcctacccgttcgccgccgccgcgatcctgaccggg
gggctgcccgcgtggctggtggtcctgggcgtcgtgttcttcctggtgccctacaacctggccatgtacggcatcaatgacgtgttcga
cttcgcctcggacctgcgcaacccccgcaaggggggtgtggagggctccgtgctgggcgaccccgcggtgcgccgccgggtgctggc
gtggtcggtgctgctgcccgtgccgttcgtggccgtgctcgcgggctggtccgccgtgcggggcgagtgggccgccgtgctggtgctc
gcggtgagcctgttcgcggtggtggcgtactcctgggcggggctgcggttcaaggagcggcccttcctggacgccgccacctccgcc
acccacttcgtctcccccgcggtctacggcctcgcgctggccggggcgacccccacgcccgccctggcggcgctgctgggggcgttc
ttcctgtggggcatggcctcgcagatgttcggggcggtgcaggacgtggtgccggaccgggaggggggcctggcctcggtggccac
cgtgctgggcgctcggcgcaccgtcctgctcgccgccggcctgtacgcggcggcgggcctgctgctgctggccaccgacccgccgg
gcccgctcgcggcgctgctggccgtgccctacgtggtgaacaccctgcgcttccgccgcatcacggacgccacctcgggcgcggcc
caccgcggctggcagctgttccttccgctgaactacgtgaccggcttcctcgtgaccctgctgctgatcgggtgggcgctgacccggg
gggcggcggcatga
SEQ ID NO: 7 - C.glutamicum crtEb nucleotide sequence
atgatggaaaaaataagactgattctattgtcatctcgccccattagctgggtcaataccgcctacccttttgggctggcatacctat
taaatgcaggagagattgactggctgttttggctaggcatcgtgttttttcttatcccgtataacatcgccatgtatggcatcaacgat
gtttttgattacgaatctgacatacgtaatccccgcaaaggcggcgtcgagggggccgtgctcccgaaaagttcccacagcacactgtt
atgggcatcggctatctcaacaattcctttcctagttattcttttcatatttggcacctggatgtcgtctttatggctgacaatct
cagtgctagcagtgattgcttattcagcaccgaaattgcgttttaaagaacgcccctttatcgatgctctaacatcttcta
ctcacttcacttcacctgcattaatcggtgcaacgatcactggaacatctccttcagcagcgatgtggatagcactgggatccttt
ttcttgtggggcatggccagtcagatccttggagcagtacaggatgttaatgcagaccgggaagctaatctgagctcaattgcc
actgtaattggggcgcgtggagccattcggctatcagtagtactttatttactagctgctgttttagtcactactttgcc
taatccggcgtggatcatcgggattgcgattctaacttacgtatttgatgcattttggaacattacagatgccagttgtga
acaggctaatcgcagttggaaagttttcctgtggctgaactactttggtgataacgatactgttaatagcaattcatcagatataa
SEQ ID NO: 8 - M.luteus NCTC2665 CrtE2 polypeptide sequence
MIRTLFWVSRPVSWVNTAYPFAAAAILTGGLPAWLWLGWFFLVPYNLAMYGINDVFDFASDL
RNPRKGGVEGSVLGDPAVRRRVLAWSVLLPVPFVAVLAGWSAVRGEWAAVLVLAVSLFAWA
YSWAGLRFKERPFLDAATSATHFVSPAVYGLALAGATPTPALAALLGAFFLWGMASQMFGAV
QDWPDREGGLASVATVLGARRTVLLAAGLYAAAGLLLLATDPPGPLAALLAVPYVVNTLRFRR
ITDATSGAAHRGWQLFLPLNYVTGFLVTLLLIGWALTRGAAA
SEQ ID NO: 9 - C.glutamicum CrtEb polypeptide sequence
MMEKIRLILLSSRPISWVNTAYPFGLAYLLNAGEIDWLFWLGIVFFLIPYNIAMYGINDVFDYESDI
RNPRKGGVEGAVLPKSSHSTLLWASAISTIPFLVILFIFGTWMSSLWLTISVLAVIAYSAPKLRFK
ERPFIDALTSSTHFTSPALIGATITGTSPSAAMWIALGSFFLWGMASQILGAVQDVNADREANLS
SIATVIGARGAIRLSWLYLLAAVLVTTLPNPAWIIGIAILTYVFDAARFWNITDASCEQANRSWKV
FLWLNYFVGAVITILLIAIHQI
SEQ ID NO: 10 - M.luteus Otnes7 crtE2 nucleotide sequence
atgatccgcaccctcttctgggcgtcccggccggtcagctgggtgaacacggcgtacccgttcgccgccgccgcgatcctgaccggg
gggctgcccgcgtggctggtggtcctgggcgtcgtgttcttcctcgtgccctacaacctggccatgtacggcatcaatgacgtgttcga
cttcgcctcggacctgcgcaacccccgcaaggggggcgtggagggctccgtgctgggcgaccccgcggtgcgccgccgggtgctggt
gtggtcggtgctgctgcccgtcccgttcgtggccgtgctcgcgggctggtccgccgtgcggggcgagtgggccgccgtgctggtgctg
gcggtgagcctgttcgcggtggtggcgtactcctgggcggggctgcggttcaaggagcggcccttcctggacgccgcgacctccgcc
acccacttcgtctcccccgcggtctacggcctcgtgctggccggggcgacccccacgcccgccctggcggcgctgctgggggccttct
tcctgtggggcatggcctcgcagatgttcggggcggtgcaggacgtggtgccggaccgggaggggggcctggcctcggtggccac
cgtgctgggcgctcggcgcaccgtcctgctcgccgccggcctgtacgcggcggcgggcctgctgctgctggccaccgacccgccgg
gcccccttgcggcgctgctggccgtgccctacgtggtgaacaccctgcgcttccgccgcatcacggacgccacctcgggcgcggcc
caccgcggctggcagctgttcctccccctgaactacgtgaccggcttcctcgtgaccctgctgctgatcgggtgggcgctgacccggg
gggcggcggcatga
SEQ ID NO: 11 - M.luteus Otnes7 CrtE2 polypeptide sequence
MIRTLFWASRPVSWVNTAYPFAAAAILTGGLPAWLWLGWFFLVPYNLAMYGINDVFDFASDL
RNPRKGGVEGSVLGDPAVRRRVLVWSVLLPVPFVAVLAGWSAVRGEWAAVLVLAVSLFAVVA
YSWAGLRFKERPFLDAATSATHFVSPAVYGLVLAGATPTPALAALLGAFFLWGMASQMFGAV
QDWPDREGGLASVATVLGARRTVLLAAGLYAAAGLLLLATDPPGPLAALLAVPYVVNTLRFRR
ITDATSGAAHRGWQLFLPLNYVTGFLVTLLLIGWALTRGAAA
SEQ ID NO: 12 - M.luteus Otnes7 crtYq nucleotide sequence
atgatctacctgctggccctgctgggtgtcatcggctgcatgctgctggtggaccggcgcttcgagctgttcctgtggcatcgcccgctc
ccggcgctgctggtgctggccgccggggtggcctacttcgtcgcctgggacctgtgggggatcgccgaaggcgtgttcctgcaccggc
agtcgccctacgtgaccggggtgatgctcgccccccagctgcccctggaggaggggttcttcctgctcttcctcagccagatcacgatg
gtgctgttcaccggggcgctgcgcctgctgcgcggccggggacgcgacgcccgtgccgcgacgccggccgatccgaccgacggg
gggagccggtga
SEQ ID NO: 13 - M.luteus Otnes7 CrtYq polypeptide sequence
MIYLLALLGVIGCMLLVDRRFELFLWHRPLPALLVLAAGVAYFVAWDLWGIAEGVFLHRQSPYV
TGVMLAPQLPLEEGFFLLFLSQITMVLFTGALRLLRGRGRDARAATPADPTDGGSR
SEQ ID NO: 14 - M.luteus Otnes7 crtYh nucleotide sequence
gtgaccttcctcgacctcgtcctcgtcttcgtgggcttcgccctggccgtgctcgtgggcgccgccctcgtcggccgcgtgcggggcgag
cacctgcgggccgtggcggccaccctggtggccctgtgggccctcacggcggtcttcgacaacgtgatgatcgccgcggggctcttc
gactacggccatgagctgctggtgggtgcctacgtgggccaggcgcccgtggaggacttcgcctacccgctcggctccgccctgctg
ctgccggcgctctggctgctgctgacgagccgtggtcgtgccggtcggcgcggccctcggccgggacgccgcccccacccggacg
atcgctga
SEQ ID NO: 15 - M.luteus Otnes7 CrtYh polypeptide sequence
VTFLDLVLVFVGFALAVLVGAALVGRVRGEHLRAVAATLVALWALTAVFDNVMIAAGLFDYGHE
LLVGAYVGQAPVEDFAYPLGSALLLPALWLLLTSRGRAGRRGPRPGRRPHPDDR
SEQ ID NO: 16 - M.luteus NCTC2665 crtX nucleotide sequence
gtgaccccggcccgccccacggtctccgtggtcgtcccggtgctcgacgacgccgagcacctgcgcgtgtgcctcgcgctgctggcc
gcccagagccggccggcgctggaggtggtggtggtggacaacggctgcgtggacgactcggcggtgctcgcccgcgccgccggc
gcgcgggtggtgcgcgagccgcgccgcggggtcccggccgcggcggccgccggcctggacgccgcggtcggggagctgctggt
gcgctgcgacgccgacacgcggatgcccgcggactggctcgaacggatcgtggcccggttcgacgccgaccccgggctcgacgc
cctcaccgggccggggaccttccacgaccagcccggcctccggggacaggtgcgggcggcgctctacaccggcacgtaccgctg
gggggcgggcgccgcggtggcggccacccccgtctggggctccaactgcgccctgcgcgccgaggcgtggcaggctgtgcggac
ccgcgtccaccgcgaacgcggggacgtgcacgatgacctggacctgtccttccagctggccctggccggccgccggatccggttcg
atccggacctgcgggtggaggtcgccgggcgcatcttccactccctgcgccagcgggtgcggcagggccggatggcggtcaccac
cctgcaggtcaactgggcccgactgtcccccgggcggcgttggctgcgccgggcggcccgggcacacccccggtcccgctggggg
cgtggccccgacggtcagtcccgggactga
SEQ ID NO: 17 - M.luteus NCTC2665 CrtX polypeptide sequence
VTPARPTVSWVPVLDDAEHLRVCLALLAAQSRPALEWWDNGCVDDSAVLARAAGARVVRE
PRRGVPAAAAAGLDAAVGELLVRCDADTRMPADWLERIVARFDADPGLDALTGPGTFHDQPG
LRGQVRAALYTGTYRWGAGAAVAATPVWGSNCALRAEAWQAVRTRVHRERGDVHDDLDLSF
QLALAGRRIRFDPDLRVEVAGRIFHSLRQRVRQGRMAVTTLQVNWARLSPGRRWLRRAARAH
PRSRWGRGPDGQSRD
SEQ ID NO: 18 - M.luteus NCTC2665 crtE nucleotide sequence
atgggtgaagcgaggacgggcggcgaggccgcgctctccggggtgaccgccgagctggacgccgcgctccgacacgccgcgg
cccaggcgcccggatccgccgccttcgccgagctgctcgactcgctccacgtccatgtgggcgccggcaagctcatccgcccccgtc
tcgtcgagctcggctggcgcctggcgaccgccgacccggtccctccgtccggccgcgctgccgtcgaccgactcggggccgccttc
gaactgctgcacaccgcgctgctcgtccacgacgacgtcatcgatcgggacgtgctgcggcgcggccagcccgccgtgcacgcctc
cgcccggcaccgcctcgaggcccgcggggtgcccgccgcggacgccgcccacgccggggtcgccgtcgccctcatcgcggggg
acgtcctgctcacccaggcgttccggctcgccgccacctgtgccgccgacaccgcccgggccgccgaggccgccgccgtcgtcttc
gacgccgccgccgtgactgcggccggcgagctcgaggacgtgctcctggggctgtcccgccacaccggtgaggagcccgatccc
gaccgcatcctcgccatgcaacggctcaagacggcgcactacacggtcggcgcgcccctgcgcgccggcgccctcctggccggg
gcggatcccgacctcgcccgggcgatgggcgaggccggcgccgacctcggcgccgcctaccaggtgatcgacgacgtcctcggc
gtgttcggcgatcccggggagaccggcaagtccgccgacggcgacctgcgcgagggcaaggccaccgtgctcaccgcccacgg
ccgccgcatccccgccgtccgcgccctgctcgacgcgggcccggccacccccgcggacatcgaggccgcccgccgcgccctcga
ggcggccggtgcccgggagcacgccctcgacgtcgccgccgagctcaccgtccgcgcccgcgagcgcatcgcggccctgcccct
ggacgagacggtccgggcggagttcgccgacgcctgccacgccgtgctgacccggaggtcctga
SEQ ID NO: 19 - M.luteus NCTC2665 CrtE polypeptide sequence
MGEARTGGEAALSGVTAELDAALRHAAAQAPGSAAFAELLDSLHVHVGAGKLIRPRLVELGWR
LATADPVPPSGRAAVDRLGAAFELLHTALLVHDDVIDRDVLRRGQPAVHASARHRLEARGVPA
ADAAHAGVAVALIAGDVLLTQAFRLAATCAADTARAAEAAAVVFDAAAVTAAGELEDVLLGLSR
HTGEEPDPDRILAMQRLKTAHYTVGAPLRAGALLAGADPDLARAMGEAGADLGAAYQVIDDVL
GVFGDPGETGKSADGDLREGKATVLTAHGRRIPAVRALLDAGPATPADIEAARRALEAAGARE
HALDVAAELTVRARERIAALPLDETVRAEFADACHAVLTRRS
SEQ ID NO: 20 - M.luteus NCTC2665 crtB nucleotide sequence
atggccgcgcccaccccgagccctgccgcgctgtacacgcggacggcccacaccgcagcggcccaggtgatccgccgctactcc
acgtccttctcctgggcctgccgcaccctgccccggcaggcacgccaggacgtggccacgatctacgccatggtccgcgtcgccga
cgaggtggtcgacggcgtcgcggtggccgccgggctcgacgaggccggggtccgcgccgccctggacgactacgagcgggcgt
gtgaggccgcgatggcgtcgggcttcgccaccgacccggtcctgcacgccttcgccgacgtggcccgtcgccacggcatcaccccg
gagctgacccgtcccttcttcgcctccatgcgcgcggacctggggatccgcgagcacggcgccgagtccctggacgcctacatccac
ggctcggccgaggtggtggggctgatgtgcctgcaggtcttcctctccctccccggcacgcgggcccggaccccgggccagcggca
ggagctgcgcgcgcaggcctcccggctgggggcggcgttccagaaggtcaacttcctcagggacctggccgcggaccaccacga
gctgggccgcacctacctgcccggtgccgcaccgggcgtgctcaccgaggcccgcaaggccgagctcgtggccgaggtccgcgc
cgacctcgacgccgccctgcccggcatccgtgtcctggaccccggggccgggcgcgccgtggccctggcgcacggactgttcgcg
gccctggtggaccggatcgaggcgaccccggcggccgagctggcccaccgccgtgtccgggtgccggaccatcagaaggcccg
gatcgccgcccgcgtcctggcacggggccgccggggaggccgccgatga
SEQ ID NO: 21 - M.luteus NCTC2665 CrtB polypeptide sequence
MAAPTPSPAALYTRTAHTAAAQVIRRYSTSFSWACRTLPRQARQDVATIYAMVRVADEVVDGV
AVAAGLDEAGVRAALDDYERACEAAMASGFATDPVLHAFADVARRHGITPELTRPFFASMRAD
LGIREHGAESLDAYIHGSAEWGLMCLQVFLSLPGTRARTPGQRQELRAQASRLGAAFQKVNF
LRDLAADHHELGRTYLPGAAPGVLTEARKAELVAEVRADLDAALPGIRVLDPGAGRAVALAHGL
FAALVDRIEATPAAELAHRRVRVPDHQKARIAARVLARGRRGGRR
SEQ ID NO: 22 - M.luteus NCTC2665 crtl nucleotide sequence
atgagcgcccgggacaccgctctcggcccgcgcaccgtggtggtgggcggcggtttcgccggactggccacggcgggcctgttggc
ccgcgacgggcaccgggtgacgctgctggagcgcggcgccgtcctgggcggccgtgccggacgctggtccgaggcggggttcac
cttcgataccgggccctcctggtacctgatgcccgaggtgatcgaccgctggttccgcctcatggggacctccgccgccgaacggctg
gacctgcgccgtctggaccccggctaccgggtgtacttcgaggggcacctccacgagccccccgtggacgtgcgcaccggccacg
cggagacgctgttcgagtccctcgagcccggcgccgggcgccggctgcgggcctacctcgactccgcgtcccggatctacgggctc
gccaaggagcacttcctctacacggacttccgccggccggccgccctggcccacccggacgtcctgcgcgccctgccggccctcgg
gccccagctgctggggggcctgcgctcccacgtcgcggcccgcttccaggacccccggctgcgccagatcctgggctacccggcg
gtcttcctcggcacgtcccccgaccgtgcccccgccatgtaccacctgatgtcccatctggacctcgccgacggcgtgcagtaccccct
cggcgggttcgcggccctcgtggacgccatggcggaggtcgtgcgcgaggccggcgtggagatccgcaccggggtcgaggcgac
cgccgtggaggtcgcggaccgtcccgcccccgccggccgcctcggacgcctggccgcccgcctgcccaggccgggagcagccc
gcggggacgagggccgacgtcgccgcccgggccgggtgaccggcgtcgcctggcggtccgacgacggcgccgcgggacgcct
cgacgccgatgtggtggtggccgccgcggacctgcaccacgtgcagacccgtctgctgcctcccggccggcgcgtcgcggagtcc
acgtgggaccggcgcgaccccggcccctccggcgtgctcgtgtgcgtgggggtgcgcggatccctgccccagctggcccatcacac
cctgctgttcacggcggactgggaggacaacttcgggcgcatcgagcggggggaggacctcgccgcggacacgtcgatctacgtct
cgcgcacctccgccacggacccgggcgtggccccggagggcgacgagaacctcttcatcctcgtcccggcccccgccgagccgg
ggtgggggcgcggcggcatccgggtccgtgacggccagggctggcgggtggaccgcgccggggacgcccaggtggaggccgt
ggcggaccgggccctcgatcagctggcccgctgggccgggatccccgacctggccgagcgcatcgtggtgcggcgcacctacgg
gcccggtgacttcgccgcggacgtgcacgcctggcggggttcgctgctgggccccgggcacacgctggcgcagtcggccatgttcc
gcccctcggtgcgggacgcggacgtggccggcctgatgtacgcgggctcctcggtgcgcccgggaatcggggtgcccatgtgcctg
atctccgccgaagtggtccgggacgaactgcgccacgacgcgcgcagggcccggcccgcgggccccggggggagcggcacat
ga
SEQ ID NO: 23 - M.luteus NCTC2665 Crtl polypeptide sequence
MSARDTALGPRTVVVGGGFAGLATAGLLARDGHRVTLLERGAVLGGRAGRWSEAGFTFDTG
PSWYLMPEVIDRWFRLMGTSAAERLDLRRLDPGYRVYFEGHLHEPPVDVRTGHAETLFESLEP
GAGRRLRAYLDSASRIYGLAKEHFLYTDFRRPAALAHPDVLRALPALGPQLLGGLRSHVAARF
QDPRLRQILGYPAVFLGTSPDRAPAMYHLMSHLDLADGVQYPLGGFAALVDAMAEVVREAGV
EIRTGVEATAVEVADRPAPAGRLGRLAARLPRPGAARGDEGRRRRPGRVTGVAWRSDDGAA
GRLDADWVAAADLHHVQTRLLPPGRRVAESTWDRRDPGPSGVLVCVGVRGSLPQLAHHTLL
FTADWEDNFGRIERGEDLAADTSIYVSRTSATDPGVAPEGDENLFILVPAPAEPGWGRGGIRV
RDGQGWRVDRAGDAQVEAVADRALDQLARWAGIPDLAERIVVRRTYGPGDFAADVHAWRGS
LLGPGHTLAQSAMFRPSVRDADVAGLMYAGSSVRPGIGVPMCLISAEVVRDELRHDARRARP
AGPGGSGT
SEQ ID NO: 24 - M.luteus NCTC2665 ORF1 nucleotide sequence
gtgccgatcggcgcggccgtcggccgggacgccgcccccacccggacgatcgctgacatgctgccgttgatccccgcagacctgct
gcgcgcgctcggcctgatcctcgtcccggtcgcggcggtgcacgccggatggccgtccgcggcggcgatgctgctcgtgttcggctc
ccagtggctcacccgctggctcgccccgggcggcgccctggactgggccgcgcaggcggtcctgctgctggccgggtggctgagc
gtcatcggcctctacccgcgggtgccgtggctggacctgctcgtgcacgccgccgcctccgccgtggtcgcctgtctgacggcactg
gtggtgggggcgtggctccggcgtcgggggaccgaggccgggcaggccgtggcgctgctcggcccgggcctggccgggctggggat
cgcggccgccgccgtggccctgggcgtggtgtgggagctggccgaatggtgggggcacacggcggtgaccccggagatcggcgt
gggctacacggacaccatcggcgacctcgccgccgatctcgtcggcgccggggtcggcgccgccctcgccgtgtgccgggggcgc
acccggtga
SEQ ID NO: 25 - M.luteus NCTC2665 ORF1 polypeptide sequence
VPIGAAVGRDAAPTRTIADMLPLIPADLLRALGLILVPVAAVHAGWPSAAAMLLVFGSQWLTRW
LAPGGALDWAAQAVLLLAGWLSVIGLYPRVPWLDLLVHAAASAVVACLTALWGAWLRRRGTE
AGQAVALLGPGLAGLGIAAAAVALGWWELAEWWGHTAVTPEIGVGYTDTIGDLAADLVGAC
GAALAVCRGRTR
SEQ ID NO: 26 - M.luteus Otnes7 Sarcinaxanthin gene cluster
      1 atgggtgaag cgaggacggg cggcgaggcc gcgctctccg gggtgaccgc cgagctggac
    61 gccgcgctcc gacatgccgc ggcccaggca cccggatccg ccgccttcgc cgagctgctc
  121 gactcgctcc acgtccatgt gggcgccggc aagctcatcc gcccccgtct cgtcgagctc
  181 ggctggcgcc tggcgaccgc cgacccggtc cctccgtccg gccgcgctgc cgtcgaccga
  241 ctcggggccg ccttcgaact gctgcacacc gcgctgctcg tccacgacga cgtcatcgat
  301 cgggacgtgc tgcggcgcgg ccagcccgcc gtgcacgcct ccgcccggca ccgcctcgag
  361 gcccgcgggg tgcccgccgc ggacgccgcc cacgccgggg tcgccgtcgc cctcatcgcg
  421 ggggacgtcc tgctcaccca ggcgttccgg ctcgccgcca cctgtgccgc cgacaccgcc
  481 cgggccgccg aggccgccgc cgtcgtcttc gacgccgccg ccgtgaccgc ggccggcgag
  541 ctcgaagacg tgctcctggg gctgtcccgc cacaccggtg aggagcccga tcccgaccgc
  601 atcctcgcca tgcaacggct caagacggcg cactacacgg tcggcgcgcc cctgcgcgcc
  661 ggcgccctcc tggccggggc ggatcccgac ctcgcccggg cgatgggcga ggccggcgcc
  721 gacctcggcg ccgcctacca ggtgatcgac gacgtcctcg gcgtgttcgg cgatcccggg
  781 gagaccggca agtccgccga cggcgacctg cgcgagggca aggccaccgt gctcaccgcc
  841 cacggccgcc tcatccccgc cgtccgcgcc ctgctcgacg cgggcccggc cacccccgcg
  901 gacatcgagg ccgcccgccg cgccctcgag gcggccggtg cccgggagca cgccctcgac
  961 gtcgccgccg agctcaccgt ccgcgcccgc gagcgcatcg cggccctgcc cctggacgag
1021 acggtccggg cggagttcgc cgacgcctgc cacgccgtgc tgacccggag gtcctgagat
1081 ggccgcgccc accccgagcc ctgccgcgct gtacacgcgg acggcccaca ccgcagcggc
1141 ccaggtgatc cgccgctact ccacgtcctt ctcctgggcc tgccgcaccc tgccccggca
1201 ggcacgccag gacgtggcca cgatctacgc catggtccgc gtcgccgacg aggtggtcga
1261 cggcgtcgcg gtggccgccg ggctcgacga ggccggggtc cgcgccgccc tggacgacta
1321 cgagcgggcg tgtgaggctg cgatggcgtc gggcttcgcc accgacccgg tcctgcacgc
1381 cttcgccgac gtggcccgtc gccacggcat caccccggag ctgacccgtc ccttcttcgc
1441 ctccatgcgc gcggacctgg ggatccgcga gcacggcgcc gagtcgctgg acgcctacat
1501 ccacggctcg gccgaggtgg tggggctgat gtgcctgcag gtcttcctct ccctccccgg
1561 cacgcgggcc cggaccccgg gccagcggca ggagctgcgc gcgcaggcct cccggctggg
1621 ggcggcgttc cagaaggtca acttcctcag ggacctggcc gcggaccacc acgagctggg
1681 ccgcacctac ctgcccggtg ccgcaccggg cgtgctcacc gaggcccgca aggccgagct
1741 cgtggccgag gtccgcgccg acctcgacgc cgccctgccc ggcatccgtg tcctggaccc
1801 cggggccggg cgcgccgtgg ccctggcgca cggactgttc gcggccctgg tggaccggat
1861 cgaggcgacc ccggcggccg agctggccca ccgccgtgtc cgggtgccgg accatcagaa
1921 ggcccggatc gccgcccgcg tcctggcacg gggccgccgg ggaggccgcc gatgagcgcc
1981 cgggacaccg ctctcggccc gcgcaccgtg gtggtgggcg gcggtttcgc cggactggcc
2041 acggcgggcc tgttggcccg cgacgggcac cgggtgacgc tgctggagcg cggcgccgtc
2101 ctgggcggcc gtgccggacg ctggtctgag gcggggttca ccttcgatac cgggccctcc
2161 tggtacctga tgcccgaggt gatcgaccgc tggttccgcc tcatggggac ctccgccgcc
2221 gaacggctgg acctgcgccg tctggacccc ggctaccggg tgtacttcga ggggcacctc
2281 cacgagcccc ccgtggacgt gcgcaccggc cacgcggaga cgctgttcga gtccctcgag
2341 cccggcgccg ggcgccggct gcgggcctac ctcgactccg cgtcccggat ctacgggctc
2401 gccaaggagc acttcctcta cacggacttc cgccggccgg ccgccctggc ccacccggac
2461 gtcctgcgcg ccctgccggc cctcgggccc cagctgctgg ggggcctgcg ctcccacgtg
2521 gcggcccgct tccaggatcc ccggctgcgc cagatcctgg gctacccggc ggtcttcctc
2581 ggcacgtccc ccgaccgtgc ccccgccatg taccacctga tgtcccatct ggacctcgcc
2641 gacggcgtgc agtaccccct cggcgggttc gcggccctcg tggacgccat ggcggaggtc
2701 gtgcgcgagg ccggcgtgga gatccgcacc ggggtcgagg cgaccgccgt cgaggtggtg
2761 gaccgtcccg cccccgccgg ccgcctcgga cgcctggccg cccgcctgcc caggccggga
2821 gcagcccgcg gggacgaggg ccgacgtcgc cgcccgggcc aggtgaccgg cgtcgcctgg
2881 cggtccgacg acggcgccgc gggacgcctc gacgccgatg tggtggtggc cgccgcggac
2941 ctgcaccacg tgcagacccg tctgctgcct cccggccggc gcgtcgcgga gtccacgtgg
3001 gaccggcgcg accccggccc ctccggcgtg ctcgtgtgcg tgggggtgcg cggatccctg
3061 ccccagctgg cccatcacac cctgctgttc acggcggact gggaggacaa cttcgggcgc
3121 atcgagcggg gagaggacct cgccgcggac acgtcgatct acgtctcgcg cacctccgcc
3181 acggacccgg gcgtggcccc ggagggcgac gagaacctct tcatcctcgt cccggccccc
3241 gccgagccgg ggtgggggcg cggcggcatc cgggtccgtg acggcgaggg ctggcgggtg
3301 gaccgcgccg gggacgccca ggtggaggcc gtggcggacc gggccctcga ccagctggcc
3361 cgctgggccg ggatcccgga cctggccgag cgcatcgtgg tgcggcgcac ctacgggccc
3421 ggtgacttcg ccgcggacgt gcacgcctgg cggggttcgc tgctgggccc cgggcacacg
3481 ctggcgcagt cggccatgtt ccgtccctcg gtgcgggacg cggacgtggc cggcctgatg
3541 tacgcgggct cctcggtgcg cccgggcatc ggggtgccca tgtgtctgat ctccgccgaa
3601 gtggtccggg acgaactgcg ccacgacgcg cgcagggccc ggcccgcggg ccccgggggg
3661 agcggcacat gatccgcacc ctcttctggg cgtcccggcc ggtcagctgg gtgaacacgg
3721 cgtacccgtt cgccgccgcc gcgatcctga ccggggggct gcccgcgtgg ctggtggtcc
3781 tgggcgtcgt gttcttcctc gtgccctaca acctggccat gtacggcatc aatgacgtgt
3841 tcgacttcgc ctcggacctg cgcaaccccc gcaagggggg cgtggagggc tccgtgctgg
3901 gcgaccccgc ggtgcgccgc cgggtgctgg tgtggtcggt gctgctgccc gtcccgttcg
3961 tggccgtgct cgcgggctgg tccgccgtgc ggggcgagtg ggccgccgtg ctggtgctgg
4021 cggtgagcct gttcgcggtg gtggcgtact cctgggcggg gctgcggttc aaggagcggc
4081 ccttcctgga cgccgcgacc tccgccaccc acttcgtctc ccccgcggtc tacggcctcg
4141 tgctggccgg ggcgaccccc acgcccgccc tggcggcgct gctgggggcc ttcttcctgt
4201 ggggcatggc ctcgcagatg ttcggggcgg tgcaggacgt ggtgccggac cgggaggggg
4261 gcctggcctc ggtggccacc gtgctgggcg ctcggcgcac cgtcctgctc gccgccggcc
4321 tgtacgcggc ggcgggcctg ctgctgc tg gccaccgacc cgccgggccc ccttgcggcg
4381 ctgctggccg tgccctacgt ggtgaacacc ctgcgcttcc gccgcatcac ggacgccacc
4441 tcgggcgcgg cccaccgcgg ctggcagctg ttcctccccc tgaactacgt gaccggcttc
4501 ctcgtgaccc tgctgctgat cgggtgggcg ctgacccggg gggcggcggc atgatctacc
4561 tgctggccct gctgggtgtc atcggctgca tgctgctggt ggaccggcgc ttcgagctgt
4621 tcctgtggca tcgcccgctc ccggcgctgc tggtgctggc cgccggggtg gcctacttcg
4681 tcgcctggga cctgtggggg atcgccgaag gcgtgttcct gcaccggcag tcgccctacg
4741 tgaccggggt gatgctcgcc ccccagctgc ccctggagga ggggttcttc ctgctcttcc
4801 tcagccagat cacgatggtg ctgttcaccg gggcgctgcg cctgctgcgc ggccggggac
4861 gcgacgcccg tgccgcgacg ccggccgatc cgaccgacgg ggggagccgg tgaccttcct
4921 cgacctcgtc ctcgtcttcg tgggcttcgc cctggccgtg ctcgtgggcg ccgccctcgt
4981 cggccgcgtg cggggcgagc acctgcgggc cgtggcggcc accctggtgg ccctgtgggc
5041 cctcacggcg gtcttcgaca acgtgatgat cgccgcgggg ctcttcgact acggccatga
5101 gctgctggtg ggtgcctacg tgggccaggc gcccgtggag gacttcgcct acccgctcgg
5161 ctccgccctg ctgctgccgg cgctctggct gctgctgacg agccgtggtc gtgccggtcg
5221 gcgcggccct cggccgggac gccgccccca cccggacgat cgctgagcgg ccgcaaaaaa
5281 atcactagtg cggccgcctg caggtcgacc atatgggaga gctcccaacg cgttggatgc
5341 atagcttgag tattctatag tgtcacctaa atagctggcg
SEQ ID NO: 27 - M.luteus Otnes7 crtE nucleotide sequence
atgggtgaagcgaggacgggcggcgaggccgcgctctccggggtgaccgccgagctggacgccgcgctccgacatgccgcggc
ccaggcacccggatccgccgccttcgccgagctgctcgactcgctccacgtccatgtgggcgccggcaagctcatccgcccccgtct
cgtcgagctcggctggcgcctggcgaccgccgacccggtccctccgtccggccgcgctgccgtcgaccgactcggggccgccttcg
aactgctgcacaccgcgctgctcgtccacgacgacgtcatcgatcgggacgtgctgcggcgcggccagcccgccgtgcacgcctcc
gcccggcaccgcctcgaggcccgcggggtgcccgccgcggacgccgcccacgccggggtcgccgtcgccctcatcgcggggga
cgtcctgctcacccaggcgttccggctcgccgccacctgtgccgccgacaccgcccgggccgccgaggccgccgccgtcgtcttcg
acgccgccgccgtgaccgcggccggcgagctcgaagacgtgctcctggggctgtcccgccacaccggtgaggagcccgatcccg
accgcatcctcgccatgcaacggctcaagacggcgcactacacggtcggcgcgcccctgcgcgccggcgccctcctggccgggg
cggatcccgacctcgcccgggcgatgggcgaggccggcgccgacctcggcgccgcctaccaggtgatcgacgacgtcctcggcg
tgttcggcgatcccggggagaccggcaagtccgccgacggcgacctgcgcgagggcaaggccaccgtgctcaccgcccacggc
cgcctcatccccgccgtccgcgccctgctcgacgcgggcccggccacccccgcggacatcgaggccgcccgccgcgccctcgag
gcggccggtgcccgggagcacgccctcgacgtcgccgccgagctcaccgtccgcgcccgcgagcgcatcgcggccctgcccctg
gacgagacggtccgggcggagttcgccgacgcctgccacgccgtgctgacccggaggtcctga
SEQ ID NO: 28 - M.luteus Otnes7 CrtE polypeptide sequence
MGEARTGGEAALSGVTAELDAALRHAAAQAPGSAAFAELLDSLHVHVGAGKLIRPRLVELGWR
LATADPVPPSGRAAVDRLGAAFELLHTALLVHDDVIDRDVLRRGQPAVHASARHRLEARGVPA
ADAAHAGVAVALIAGDVLLTQAFRLAATCAADTARAAEAAAVVFDAAAVTAAGELEDVLLGLSR
HTGEEPDPDRILAMQRLKTAHYTVGAPLRAGALLAGADPDLARAMGEAGADLGAAYQVIDDVL
GVFGDPGETGKSADGDLREGKATVLTAHGRLIPAVRALLDAGPATPADIEAARRALEAAGARE
HALDVAAELTVRARERIAALPLDETVRAEFADACHAVLTRRS
SEQ ID NO: 29 - M.luteus Otnes7 crtB nucleotide sequence
atggccgcgcccaccccgagccctgccgcgctgtacacgcggacggcccacaccgcagcggcccaggtgatccgccgctactcc
acgtccttctcctgggcctgccgcaccctgccccggcaggcacgccaggacgtggccacgatctacgccatggtccgcgtcgccga
cgaggtggtcgacggcgtcgcggtggccgccgggctcgacgaggccggggtccgcgccgccctggacgactacgagcgggcgt
gtgaggctgcgatggcgtcgggcttcgccaccgacccggtcctgcacgccttcgccgacgtggcccgtcgccacggcatcaccccg
gagctgacccgtcccttcttcgcctccatgcgcgcggacctggggatccgcgagcacggcgccgagtcgctggacgcctacatccac
ggctcggccgaggtggtggggctgatgtgcctgcaggtcttcctctccctccccggcacgcgggcccggaccccgggccagcggca
ggagctgcgcgcgcaggcctcccggctgggggcggcgttccagaaggtcaacttcctcagggacctggccgcggaccaccacga
gctgggccgcacctacctgcccggtgccgcaccgggcgtgctcaccgaggcccgcaaggccgagctcgtggccgaggtccgcgc
cgacctcgacgccgccctgcccggcatccgtgtcctggaccccggggccgggcgcgccgtggccctggcgcacggactgttcgcg
gccctggtggaccggatcgaggcgaccccggcggccgagctggcccaccgccgtgtccgggtgccggaccatcagaaggcccg
gatcgccgcccgcgtcctggcacggggccgccggggaggccgccgatga
SEQ ID NO: 30 - M.luteus Qtnes7 CrtB polypeptide sequence
MAAPTPSPAALYTRTAHTAAAQVIRRYSTSFSWACRTLPRQARQDVATIYAMVRVADEVVDGV
AVAAGLDEAGVRAALDDYERACEAAMASGFATDPVLHAFADVARRHGITPELTRPFFASMRAD
LGIREHGAESLDAYIHGSAEWGLMCLQVFLSLPGTRARTPGQRQELRAQASRLGAAFQKVNF
LRDLAADHHELGRTYLPGAAPGVLTEARKAELVAEVRADLDAALPGIRVLDPGAGRAVALAHGL
FAALVDRIEATPAAELAHRRVRVPDHQKARIAARVLARGRRGGRR
SEQ ID NO: 31 - M.luteus Otnes7 crtl nucleotide sequence
atgagcgcccgggacaccgctctcggcccgcgcaccgtggtggtgggcggcggtttcgccggactggccacggcgggcctgttggc
ccgcgacgggcaccgggtgacgctgctggagcgcggcgccgtcctgggcggccgtgccggacgctggtctgaggcggggttcac
cttcgataccgggccctcctggtacctgatgcccgaggtgatcgaccgctggttccgcctcatggggacctccgccgccgaacggctg
gacctgcgccgtctggaccccggctaccgggtgtacttcgaggggcacctccacgagccccccgtggacgtgcgcaccggccacg
cggagacgctgttcgagtccctcgagcccggcgccgggcgccggctgcgggcctacctcgactccgcgtcccggatctacgggctc
gccaaggagcacttcctctacacggacttccgccggccggccgccctggcccacccggacgtcctgcgcgccctgccggccctcgg
gccccagctgctggggggcctgcgctcccacgtggcggcccgcttccaggatccccggctgcgccagatcctgggctacccggcgg
tcttcctcggcacgtcccccgaccgtgcccccgccatgtaccacctgatgtcccatctggacctcgccgacggcgtgcagtaccccctc
ggcgggttcgcggccctcgtggacgccatggcggaggtcgtgcgcgaggccggcgtggagatccgcaccggggtcgaggcgacc
gccgtcgaggtggtggaccgtcccgcccccgccggccgcctcggacgcctggccgcccgcctgcccaggccgggagcagcccgc
ggggacgagggccgacgtcgccgcccgggccaggtgaccggcgtcgcctggcggtccgacgacggcgccgcgggacgcctcg
acgccgatgtggtggtggccgccgcggacctgcaccacgtgcagacccgtctgctgcctcccggccggcgcgtcgcggagtccac
gtgggaccggcgcgaccccggcccctccggcgtgctcgtgtgcgtgggggtgcgcggatccctgccccagctggcccatcacaccc
tgctgttcacggcggactgggaggacaacttcgggcgcatcgagcggggagaggacctcgccgcggacacgtcgatctacgtctcg
cgcacctccgccacggacccgggcgtggccccggagggcgacgagaacctcttcatcctcgtcccggcccccgccgagccgggg
tgggggcgcggcggcatccgggtccgtgacggcgagggctggcgggtggaccgcgccggggacgcccaggtggaggccgtgg
cggaccgggccctcgaccagctggcccgctgggccgggatcccggacctggccgagcgcatcgtggtgcggcgcacctacgggc
ccggtgacttcgccgcggacgtgcacgcctggcggggttcgctgctgggccccgggcacacgctggcgcagtcggccatgttccgtc
cctcggtgcgggacgcggacgtggccggcctgatgtacgcgggctcctcggtgcgcccgggcatcggggtgcccatgtgtctgatct
ccgccgaagtggtccgggacgaactgcgccacgacgcgcgcagggcccggcccgcgggccccggggggagcggcacatga
SEQ ID NO: 32 - M.luteus Otnes7 Crtl polypeptide sequence
MSARDTALGPRTVWGGGFAGLATAGLLARDGHRVTLLERGAVLGGRAGRWSEAGFTFDTG
PSWYLMPEVIDRWFRLMGTSAAERLDLRRLDPGYRVYFEGHLHEPPVDVRTGHAETLFESLEP
GAGRRLRAYLDSASRIYGLAKEHFLYTDFRRPAALAHPDVLRALPALGPQLLGGLRSHVAARF
QDPRLRQILGYPAVFLGTSPDRAPAMYHLMSHLDLADGVQYPLGGFAALVDAMAEVVREAGV
EIRTGVEATAVEWDRPAPAGRLGRLAARLPRPGAARGDEGRRRRPGQVTGVAWRSDDGAA
GRLDADWVAAADLHHVQTRLLPPGRRVAESTWDRRDPGPSGVLVCVGVRGSLPQLAHHTLL
FTADWEDNFGRIERGEDLAADTSIYVSRTSATDPGVAPEGDENLFILVPAPAEPGWGRGGIRV
RDGEGWRVDRAGDAQVEAVADRALDQLARWAGIPDLAERIWRRTYGPGDFAADVHAWRGS
LLGPGHTLAQSAMFRPSVRDADVAGLMYAGSSVRPGIGVPMCLISAEVVRDELRHDARRARP
AGPGGSGT
SEQ ID NO: 33 - M.luteus Otnes7 CrtX nucleotide sequence
gtgaccccggcccgccccacggtctccgtggtcgtcccggtgctcgacgacgccgagcacctgcgcgtgtgcctcgccctgctggcc
gcccagagccggccggcgctggaggtggtggtggtggacaacggctgcgtggacgactcggcggtgctcgcccgcgccgccggc
gcgcgggtggtgcacgagccgcgccgcggggtcccggccgcggcggccgccggcctggacgccgcggtcggggagctgctggt
gcgctgcgacgccgacacgcggatgcccgcggactggctcgaacggatcgtggcccggttcgacgccgactccgggctcgacgc
cctcaccgggccggggaccttccacgaccagcccggcctccgggggcgggtgcgggcggcgctctacaccggcgcgtaccgctg
gggggcgggcgccgcggtggcggccacccccgtctggggctccaactgcgccctgcgcgccgaggcgtggcaggctgtacggac
ccgcgtccaccgcgagcgcggggacgtgcacgatgacctggacctgtccttccagctggccttggccggccgccggatccggttcg
atccggacctgcgggtggaggtcgccgggcgcatcttccactccctgcgccagcgggtgcggcagggccggatggcggtcaccac
cctgcaggtcaactgggcccggctgtcccccgggcggcggtggctgcgccgggcggcccgggcacgcccccggccccgctgggg
gcgtggccccgacggtcagtcccgcgactga
SEQ ID NO: 34 - M.luteus Otnes7 CrtX polypeptide sequence
VTPARPTVSWVPVLDDAEHLRVCLALLAAQSRPALEWWDNGCVDDSAVLARAAGARVVHE
PRRGVPAAAAAGLDAAVGELLVRCDADTRMPADWLERIVARFDADSGLDALTGPGTFHDQPG
LRGRVRAALYTGAYRWGAGAAVAATPVWGSNCALRAEAWQAVRTRVHRERGDVHDDLDLSF
QLALAGRRIRFDPDLRVEVAGRIFHSLRQRVRQGRMAVTTLQVNWARLSPGRRWLRRAARAR
PRPRWGRGPDGQSRD
SEQ ID NO: 35 - M.luteus Otnes7 ORF1 nucleotide sequence
gtgccggtcggcgcggccctcggccgggacgccgcccccacccggacgatcgctgacatgctgcagctgatccccgcagacctgc
agcgcgcgctcgacatgatcctcgtcccggtcgcgacggtgcacgcaggatggccgtccgcgacggcgatgctgctcgtgttcggct
cccagtggctcacccgctggctcgccccgagcggcgccctggactgggccgcgcaggcggtcctgctgctggccgggtggctgag
cgtcatcggcctctacccacgggtgccgtggctggacctgctcgtgcacgccgccgcctccgccgtggtcgcctgtctgacggcactg
gtggtgggggcatggctccggcgtcgggggaccgaggccgggcaggccgtggcgctgctcggcccgggcctggccggtctgggg
atcgcggccgccgccgtggccctgggcgtggtgtgggagctggccgaatggcgggggtacacggcggtgacccccgagatcggtg
tgggctacacggacaccatcggcgacctcgccgccgatctcgtcggcgccgggatcggcgccgccctcgccgtgcgccgggagcg
cacccggtga
SEQ ID NO: 36 - M.luteus Otnes7 ORF1 polypeptide sequence
VPVGAALGRDAAPTRTIADMLQLIPADLQRALDMILVPVATVHAGWPSATAMLLVFGSQWLTR
WLAPSGALDWAAQAVLLLAGWLSVIGLYPRVPWLDLLVHAAASAWACLTALVVGAWLRRRG
TEAGQAVALLGPGLAGLGIAAAAVALGWWELAEWRGYTAVTPEIGVGYTDTIGDLAADLVGA
GIGAALAVRRERTR
SEQ ID NO: 37 - M.luteus Otnes7 full-length Sarcinaxanthin gene cluster
atgggtgaagcgaggacgggcggcgaggccgcgctctccggggtgaccgccgagctggacgccgcgctccgacatgccgcggc
ccaggcacccggatccgccgccttcgccgagctgctcgactcgctccacgtccatgtgggcgccggcaagctcatccgcccccgtct
cgtcgagctcggctggcgcctggcgaccgccgacccggtccctccgtccggccgcgctgccgtcgaccgactcggggccgccttcg
aactgctgcacaccgcgctgctcgtccacgacgacgtcatcgatcgggacgtgctgcggcgcggccagcccgccgtgcacgcctcc
gcccggcaccgcctcgaggcccgcggggtgcccgccgcggacgccgcccacgccggggtcgccgtcgccctcatcgcggggga
cgtcctgctcacccaggcgttccggctcgccgccacctgtgccgccgacaccgcccgggccgccgaggccgccgccgtcgtcttcg
acgccgccgccgtgaccgcggccggcgagctcgaagacgtgctcctggggctgtcccgccacaccggtgaggagcccgatcccg
accgcatcctcgccatgcaacggctcaagacggcgcactacacggtcggcgcgcccctgcgcgccggcgccctcctggccgggg
cggatcccgacctcgcccgggcgatgggcgaggccggcgccgacctcggcgccgcctaccaggtgatcgacgacgtcctcggcg
tgttcggcgatcccggggagaccggcaagtccgccgacggcgacctgcgcgagggcaaggccaccgtgctcaccgcccacggc
cgcctcatccccgccgtccgcgccctgctcgacgcgggcccggccacccccgcggacatcgaggccgcccgccgcgccctcgag
gcggccggtgcccgggagcacgccctcgacgtcgccgccgagctcaccgtccgcgcccgcgagcgcatcgcggccctgcccctg
gacgagacggtccgggcggagttcgccgacgcctgccacgccgtgctgacccggaggtcctgagatggccgcgcccaccccgag
ccctgccgcgctgtacacgcggacggcccacaccgcagcggcccaggtgatccgccgctactccacgtccttctcctgggcctgccg
caccctgccccggcaggcacgccaggacgtggccacgatctacgccatggtccgcgtcgccgacgaggtggtcgacggcgtcgc
ggtggccgccgggctcgacgaggccggggtccgcgccgccctggacgactacgagcgggcgtgtgaggctgcgatggcgtcggg
cttcgccaccgacccggtcctgcacgccttcgccgacgtggcccgtcgccacggcatcaccccggagctgacccgtcccttcttcgcc
tccatgcgcgcggacctggggatccgcgagcacggcgccgagtcgctggacgcctacatccacggctcggccgaggtggtgggg
ctgatgtgcctgcaggtcttcctctccctccccggcacgcgggcccggaccccgggccagcggcaggagctgcgcgcgcaggcctc
ccggctgggggcggcgttccagaaggtcaacttcctcagggacctggccgcggaccaccacgagctgggccgcacctacctgccc
ggtgccgcaccgggcgtgctcaccgaggcccgcaaggccgagctcgtggccgaggtccgcgccgacctcgacgccgccctgccc
ggcatccgtgtcctggaccccggggccgggcgcgccgtggccctggcgcacggactgttcgcggccctggtggaccggatcgagg
cgaccccggcggccgagctggcccaccgccgtgtccgggtgccggaccatcagaaggcccggatcgccgcccgcgtcctggcac
ggggccgccggggaggccgccgatgagcgcccgggacaccgctctcggcccgcgcaccgtggtggtgggcggcggtttcgccgg
actggccacggcgggcctgttggcccgcgacgggcaccgggtgacgctgctggagcgcggcgccgtcctgggcggccgtgccgg
acgctggtctgaggcggggttcaccttcgataccgggccctcctggtacctgatgcccgaggtgatcgaccgctggttccgcctcatgg
ggacctccgccgccgaacggctggacctgcgccgtctggaccccggctaccgggtgtacttcgaggggcacctccacgagccccc
cgtggacgtgcgcaccggccacgcggagacgctgttcgagtccctcgagcccggcgccgggcgccggctgcgggcctacctcga
ctccgcgtcccggatctacgggctcgccaaggagcacttcctctacacggacttccgccggccggccgccctggcccacccggacg
tcctgcgcgccctgccggccctcgggccccagctgctggggggcctgcgctcccacgtggcggcccgcttccaggatccccggctgc
gccagatcctgggctacccggcggtcttcctcggcacgtcccccgaccgtgcccccgccatgtaccacctgatgtcccatctggacctc
gccgacggcgtgcagtaccccctcggcgggttcgcggccctcgtggacgccatggcggaggtcgtgcgcgaggccggcgtggag
atccgcaccggggtcgaggcgaccgccgtcgaggtggtggaccgtcccgcccccgccggccgcctcggacgcctggccgcccgc
ctgcccaggccgggagcagcccgcggggacgagggccgacgtcgccgcccgggccaggtgaccggcgtcgcctggcggtccg
acgacggcgccgcgggacgcctcgacgccgatgtggtggtggccgccgcggacctgcaccacgtgcagacccgtctgctgcctcc
cggccggcgcgtcgcggagtccacgtgggaccggcgcgaccccggcccctccggcgtgctcgtgtgcgtgggggtgcgcggatcc
ctgccccagctggcccatcacaccctgctgttcacggcggactgggaggacaacttcgggcgcatcgagcggggagaggacctcg
ccgcggacacgtcgatctacgtctcgcgcacctccgccacggacccgggcgtggccccggagggcgacgagaacctcttcatcctc
gtcccggcccccgccgagccggggtgggggcgcggcggcatccgggtccgtgacggcgagggctggcgggtggaccgcgccg
gggacgcccaggtggaggccgtggcggaccgggccctcgaccagctggcccgctgggccgggatcccggacctggccgagcgc
atcgtggtgcggcgcacctacgggcccggtgacttcgccgcggacgtgcacgcctggcggggttcgctgctgggccccgggcacac
gctggcgcagtcggccatgttccgtccctcggtgcgggacgcggacgtggccggcctgatgtacgcgggctcctcggtgcgcccggg
catcggggtgcccatgtgtctgatctccgccgaagtggtccgggacgaactgcgccacgacgcgcgcagggcccggcccgcgggc
cccggggggagcggcacatgatccgcaccctcttctgggcgtcccggccggtcagctgggtgaacacggcgtacccgttcgccgcc
gccgcgatcctgaccggggggctgcccgcgtggctggtggtcctgggcgtcgtgttcttcctcgtgccctacaacctggccatgtacgg
catcaatgacgtgttcgacttcgcctcggacctgcgcaacccccgcaaggggggcgtggagggctccgtgctgggcgaccccgcgg
tgcgccgccgggtgctggtgtggtcggtgctgctgcccgtcccgttcgtggccgtgctcgcgggctggtccgccgtgcggggcgagtg
ggccgccgtgctggtgctggcggtgagcctgttcgcggtggtggcgtactcctgggcggggctgcggttcaaggagcggcccttcctg
gacgccgcgacctccgccacccacttcgtctcccccgcggtctacggcctcgtgctggccggggcgacccccacgcccgccctggc
ggcgctgctgggggccttcttcctgtggggcatggcctcgcagatgttcggggcggtgcaggacgtggtgccggaccgggaggggg
gcctggcctcggtggccaccgtgctgggcgctcggcgcaccgtcctgctcgccgccggcctgtacgcggcggcgggcctgctgctgc
tggccaccgacccgccgggcccccttgcggcgctgctggccgtgccctacgtggtgaacaccctgcgcttccgccgcatcacggac
gccacctcgggcgcggcccaccgcggctggcagctgttcctccccctgaactacgtgaccggcttcctcgtgaccctgctgctgatcg
ggtgggcgctgacccggggggcggcggcatgatctacctgctggccctgctgggtgtcatcggctgcatgctgctggtggaccggcg
cttcgagctgttcctgtggcatcgcccgctcccggcgctgctggtgctggccgccggggtggcctacttcgtcgcctgggacctgtg
gatcgccgaaggcgtgttcctgcaccggcagtcgccctacgtgaccggggtgatgctcgccccccagctgcccctggaggaggggtt
cttcctgctcttcctcagccagatcacgatggtgctgttcaccggggcgctgcgcctgctgcgcggccggggacgcgacgcccgtgcc
gcgacgccggccgatccgaccgacggggggagccggtgaccttcctcgacctcgtcctcgtcttcgtgggcttcgccctggccgtgct
cgtgggcgccgccctcgtcggccgcgtgcggggcgagcacctgcgggccgtggcggccaccctggtggccctgtgggccctcacg
gcggtcttcgacaacgtgatgatcgccgcggggctcttcgactacggccatgagctgctggtgggtgcctacgtgggccaggcgccc
gtggaggacttcgcctacccgctcggctccgccctgctgctgccggcgctctggctgctgctgacgagccgtggtcgtgccggtcggc
gcggccctcggccgggacgccgcccccacccggacgatcgctgacatgctgcagctgatccccgcagacctgcagcgcgcgctc
gacatgatcctcgtcccggtcgcgacggtgcacgcaggatggccgtccgcgacggcgatgctgctcgtgttcggctcccagtggctca
cccgctggctcgccccgagcggcgccctggactgggccgcgcaggcggtcctgctgctggccgggtggctgagcgtcatcggcctc
tacccacgggtgccgtggctggacctgctcgtgcacgccgccgcctccgccgtggtcgcctgtctgacggcactggtggtgggggcat
ggctccggcgtcgggggaccgaggccgggcaggccgtggcgctgctcggcccgggcctggccggtctggggatcgcggccgccg
ccgtggccctgggcgtggtgtgggagctggccgaatggcgggggtacacggcggtgacccccgagatcggtgtgggctacacgga
caccatcggcgacctcgccgccgatctcgtcggcgccgggatcggcgccgccctcgccgtgcgccgggagcgcacccggtgacc
ccggcccgccccacggtctccgtggtcgtcccggtgctcgacgacgccgagcacctgcgcgtgtgcctcgccctgctggccgcccag
agccggccggcgctggaggtggtggtggtggacaacggctgcgtggacgactcggcggtgctcgcccgcgccgccggcgcgcgg
gtggtgcacgagccgcgccgcggggtcccggccgcggcggccgccggcctggacgccgcggtcggggagctgctggtgcgctgc
gacgccgacacgcggatgcccgcggactggctcgaacggatcgtggcccggttcgacgccgactccgggctcgacgccctcacc
gggccggggaccttccacgaccagcccggcctccgggggcgggtgcgggcggcgctctacaccggcgcgtaccgctggggggc
gggcgccgcggtggcggccacccccgtctggggctccaactgcgccctgcgcgccgaggcgtggcaggctgtacggacccgcgt
ccaccgcgagcgcggggacgtgcacgatgacctggacctgtccttccagctggccttggccggccgccggatccggttcgatccgg
acctgcgggtggaggtcgccgggcgcatcttccactccctgcgccagcgggtgcggcagggccggatggcggtcaccaccctgca
ggtcaactgggcccggctgtcccccgggcggcggtggctgcgccgggcggcccgggcacgcccccggccccgctgggggcgtg
gccccgacggtcagtcccgcgactga
```

### REFERENCES

Altschul, S. F., et al., 1997, “Gapped BLAST and PSI-BLAST: a new generation of protein database search programs”. Nucleic Acids Res. 25: 3389-3402
Blatny et al., 1997a Plasmid. 38:35-51
Blatny et al., 1997b Appl. Environ. Microbiol. 63(2):370-379
Brautaset et al., 2000 Metab. Enq. 2(2):104-114
Brautaset, T., Lale, R., and Valla, S. (2009). “Positively regulated bacterial expression systems.” Microbial Biotechnology 2: 15-30
Cunningham, F. X., Jr., D. Chamovitz, et al. (1993). “Cloning and functional expression in Escherichia coli of a cyanobacterial gene for lycopene cyclase, the enzyme that catalyzes the biosynthesis of beta-carotene.” FEBS Lett 328(1-2): 130-8
Cunningham, F. X., Jr. and E. Gantt (2007). “A portfolio of plasmids for identification and analysis of carotenoid pathway enzymes: Adonis aestivalis as a case study.” Photosynth Res 92(2): 245-59
Cunningham, F. X., Jr., Z. Sun, et al. (1994). “Molecular structure and enzymatic function of lycopene cyclase from the cyanobacterium Synechococcus sp strain PCC7942.” Plant Cell 6(8): 1107-21
Das, A., S.-H. Yoon, et al. (2007). “An update on microbial carotenoid production: application of recent metabolic engineering tools.” Applied Microbiology and Biotechnology 77(3): 505-512
Dower, W. J., J. F. Miller, et al. (1988). “High efficiency transformation of E. coli by high voltage electroporation.” Nucleic Acids Res 16(13): 6127-45
Fang, T. J. and Y. S. Cheng (1992). “Isolation of astaxanthin over-producing mutants of Phaffia rhodozyma and their fermentation kinetics.” Zhonqhua Min Guo Wei Shenq Wu Ji Mian Yi Xue Za Zhi 25(4): 209-22
Fraser, P. D. and P. M. Bramley (2004). “The biosynthesis and nutritional uses of carotenoids.” Prog Lipid Res 43(3): 228-65
Harker, M. and P. M. Bramley (1999). “Expression of prokaryotic 1-deoxy-D-xylulose-5-phosphatases in Escherichia coli increases carotenoid and ubiquinone biosynthesis.” FEBS Lett 448(1): 115-9
Holm, 1993, J. of Mol. Biology, 233: 123-38
Holm, 1995, Trends in Biochemical Sciences, 20: 478-480
Holm, 1998, Nucleic Acid Research, 26: 316-9
Kaiser, P., P. Surmann, et al. (2007). “A small-scale method for quantitation of carotenoids in bacteria and yeasts.” J Microbiol Methods 70(1): 142-9
Kim, D., J. S. Lee, Y. K. Park, J. F. Kim, H. Jeong, T. K. Oh, B. S. Kim, and C. H. Lee. 2007. Biosynthesis of antibiotic prodiginines in the marine bacterium Hahella chejuensis KCTC 2396. J. Appl. Microbiol. 102, 937-944.
Krubasik, P., M. Kobayashi, et al. (2001). “Expression and functional analysis of agene cluster involved in the synthesis of decaprenoxanthin reveals the mechanisms for C50 carotenoid formation.” Eur J Biochem 268(13): 3702-8.
Krubasik, P. and G. Sandmann (2000). “A carotenogenic gene cluster from Brevibacterium linens with novel lycopene cyclase genes involved in the synthesis of aromatic carotenoids.” Mol Gen Genet. 263(3): 423-32
Krubasik, P., S. Takaichi, et al. (2001). “Detailed biosynthetic pathway to decaprenoxanthin diglucoside in Corynebacterium glutamicum and identification of novel intermediates.” Arch Microbiol 176(3): 217-23
Kurusu, Y., M. Kainuma, et al. (1990). “Electroporation-transformation system for coryneform bacteria by auxotrophic complementation.” Agric Biol Chem 54(2): 443-7
Mermod et al., J. Bacteriol. 167(2):447-454, 1986
Myers, E. and Miller, W. 1988, “Optical Alignments in Linear Space”, CABIOS 4: 11-17
Pearson, W. R. and Lipman, D. J. 1988, “Improved tools for biological sequence analysis”, PNAS 85:2444-2448
Pearson, W. R. 1990, “Rapid and sensitive sequence comparison with FASTP and FASTA” Methods in Enzymology 183:63-98
Raja, R., S. Hemaiswarya, et al. (2007). “Exploitation of Dunaliella for beta-carotene production.” Appl Microbiol Biotechnol 74(3): 517-23
Ramos et al. FEBS Lett, 226(2):241-246, 1988
Reichenbach, H., W. Kohl, A. Böttger-Vetter, and H. Achenbach. 1980. Flexirubin-type pigments in flavobacterium. Arch. Microbiol. 126, 291-293
Rodriguez-Concepcion, M. and A. Boronat (2002). “Elucidation of the methylerythritol phosphate pathway for isoprenoid biosynthesis in bacteria and plastids. A metabolic milestone achieved through genomics.” Plant Physiol 130(3): 1079-89
Sambrook, J., E. F. Fritsch, et al. (1989). “Molecular cloning: a Laboratory Manual”, 2nd edn. Cols Spring Harbor Laboratory Press, Cold Spring Harbor, N.Y.
Sletta et al., 2004 Appl. Env. Microbiol. 70(12):7033-7039
Sletta et al., 2007 Appl. Env. Microbiol. 73(3):906-912
Stafsnes M H, J. K., Kildahl-Andersen G, Valla S, Ellingsen T E, Bruheim P. (2010). “Isolation and characterization of marine pigmented bacteria from Norwegian coastal waters and screening for carotenoids with UVA-blue light absorbing properties” The Journal of Microbiology 48(1): 16-23
Tao, L., H. Yao, et al. (2007). “Genes from a Dietzia sp. for synthesis of C40 and C50 beta-cyclic carotenoids.” Gene 386(1-2): 90-7
Thompson, J. D et al., 1994, “CLUSTAL W: Improving the sensitivity of progressive multiple sequence alignment through sequence weighting, position-specific gap penalties and weight matrix choice”. Nucleic Acids Res 22: 4673-4680
Tripathi, G. and S. K. Rawal (1998). “Simple and efficient protocol for isolation of high molecular weight DNA from Streptomyces aureofaciens.” Biotechnology Techniques 12(8): 629-631
Vertes, A. A., Y. Asai, et al. (1994). “Transposon mutagenesis of coryneform bacteria.” Mol Gen Genet. 245(4): 397-405
Winther-Larsen et al., 2000a Metab. Enq. 2:79-91
Winther-Larsen et al., 2000b Metab. Enq. 2:92-103

1. A method of producing sarcinaxanthin or a derivative thereof, said method comprising introducing into and expressing in a host cell one or more nucleic acid molecules encoding an activity in the sarcinaxanthin biosynthetic pathway, wherein said one or more nucleic acid molecules comprise:
(i) a nucleotide sequence as set forth in SEQ ID NO: 37 or a part thereof;
(ii) a nucleotide sequence with at least 90% sequence identity to SEQ ID NO: 37, or a part thereof; or
(iii) a nucleotide sequence complementary to (i) or (ii).

2. The method of claim 1, wherein said one or more nucleic acid molecules comprise:
(i) a nucleotide sequence as set forth in SEQ ID NO: 26 or a part thereof;
(ii) a nucleotide sequence with at least 90% sequence identity to SEQ ID NO: 26, or a part thereof; or
(iii) a nucleotide sequence complementary to (i) or (ii).

3. The method of claim 1, wherein said one or more nucleic acid molecules encode the sarcinaxanthin biosynthetic pathway.

4. The method of claim 1, further comprising the step of isolating the sarcinaxanthin or derivative thereof from the host cell.

5. The method of claim 1, wherein said method comprises introducing into and expressing in a host cell:
(a) one or more nucleic acid molecules comprising nucleotide sequences encoding one or more proteins capable of synthesising flavuxanthin; and
(b) one or more nucleic acid molecules comprising nucleotide sequences encoding one or more proteins having or contributing to C₅₀ carotenoid γ-cyclase activity,
wherein said one or more proteins of (b) are capable of catalysing the conversion of flavuxanthin to sarcinaxanthin.

6. The method of claim 5, wherein said host cell is a lycopene-producing host cell, preferably wherein said lycopene-producing host cell is capable of producing lycopene at levels of at least 0.5 mg/g CDW, further preferably, wherein the lycopene producing host cell comprises the plasmid pAC-LYC.

7. The method of claim 6, wherein said one or more proteins of (a) are capable of catalysing the conversion of lycopene to flavuxanthin.

8. The method of claim 7, wherein said one or more proteins have lycopene elongase activity.

9. The method of claim 5, wherein said one or more nucleic acid molecule of (b) comprises:
(1) a nucleic acid molecule encoding a C₅₀ carotenoid γ-cyclase subunit and comprising:
(i) a nucleotide sequence as set forth in all or part of SEQ ID NO: 12 or SEQ ID NO: 2, or which is degenerate therewith, or which has at least 90% sequence identity to SEQ ID NO: 12 or 2; or
(ii) a nucleotide sequence encoding a protein having all or part of an amino acid sequence as set forth in SEQ ID NO: 13 or 3 or an amino acid sequence which is at least 90% identical to SEQ ID NO: 13 or 3; and
(2) a nucleic acid molecule encoding a C₅₀ carotenoid γ-cyclase subunit and comprising:
(i) a nucleotide sequence as set forth in all or part of SEQ ID NO: 14 or 4, or which is degenerate therewith, or which has at least 90% sequence identity to SEQ ID NO: 14 or 4; or
(ii) a nucleotide sequence encoding a protein having all or part of an amino acid sequence as set forth in SEQ ID NO: 15 or 5 or an amino acid sequence which is at least 90% identical to SEQ ID NO: 15 or 5.

10. The method of claim 5, wherein said one or more nucleic acid molecules of (a) comprise:
(i) a nucleotide sequence as set forth in all or part of SEQ ID NO: 10, 6 or 7, or which is degenerate therewith, or which has at least 90% sequence identity to SEQ ID NO: 10, 6 or 7; or
(ii) a nucleotide sequence encoding a protein having all or part of an amino acid sequence as set forth in SEQ ID NO: 11, 8 or 9, or an amino acid sequence which is at least 90% identical to SEQ ID NO: 11, 8 or 9.

11. The method of claim 5 any one of claims of claims 5 to 8, wherein said one or more nucleic acid molecule comprises a nucleotide sequence encoding all or part of a protein having an amino acid sequence selected from the sequences as set forth in any one of SEQ ID NO: 11, 13 and 15 or an amino acid sequence which has at least 90% sequence identity to SEQ ID NO: 11, 13 or 15.

12. The method of claim 11, wherein said nucleotide sequence encodes a protein which when expressed in a lycopene-producing host cell together with each of the other said proteins results in at least 91% of the total carotenoids produced being sarcinaxanthin, or a nucleic acid molecule which comprises a nucleotide sequence which is the complement of any aforesaid sequence.

13. The method of claim 11, wherein said nucleotide sequence encodes a protein which when expressed in a lycopene-producing host cell together with each of the other said proteins results in sarcinaxanthin production to a level of at least 150 μg/g of cell dry weight (CDW).

14. The method of claim 1, wherein said one or more nucleic acid molecules comprise:
(i) a nucleotide sequence selected from sequences as set forth in SEQ ID NO: 10, 12 and 14;
(ii) a nucleotide sequence which is degenerate with the sequence of any one of SEQ ID NOs: 10, 12 or 14;
(iii) a nucleotide sequence which has at least 90% sequence identity to any one of SEQ ID NOs: 10, 12 or 14;
(iv) a nucleotide sequence which is a part of the nucleotide sequence of any one of SEQ ID NOs: 10, 12 or 14 or of a nucleotide sequence which is degenerate therewith; or
(v) a nucleotide sequence which is complementary to any of (i) to (iv) above.

15. The method of claim 14, wherein said one or more nucleic acid molecules comprises a nucleotide sequence encoding a protein having lycopene elongase activity and an amino acid sequence as set forth in all or part of SEQ ID NO: 11 or an amino acid sequence which is at least 90% identical to SEQ ID NO: 11, wherein said amino acid sequence comprises one or more of the following:
(a) alanine at position 8;
(b) valine at position 88;
(c) valine at position 158;
or a nucleotide sequence which is the complement of any aforesaid sequence, wherein the position numbers are stated with reference to SEQ ID NO. 11, preferably wherein the nucleic acid molecule comprises a nucleotide sequence as set forth in SEQ ID NO: 10 or a part of variant thereof, or a complement thereof.

16. The method of claim 14, wherein said one or more nucleic acid molecules comprises a nucleotide sequence encoding a protein which contributes to C₅₀ carotenoid γ-cyclase activity and which has an amino acid sequence as set forth in all or part of SEQ ID NO: 13 or an amino acid sequence which is at least 90% identical to SEQ ID NO: 13, wherein said amino acid sequence comprises one or more of the following:
(a) valine at position 44;
(b) valine at position 64;
(c) glycine at position 103;
(d) arginine at position 104;
(e) proline at position 111;
(f) glycine at position 117;
or a nucleotide sequence which is the complement of any aforesaid sequence, wherein the position numbers are stated with reference to SEQ ID NO. 13, preferably wherein the nucleic acid molecule comprises a nucleotide sequence as set forth in SEQ ID NO: 12 or a part of variant thereof, or a complement thereof.

17. The method of claim 14, wherein said one or more nucleic acid molecules comprises a nucleotide sequence encoding a protein which contributes to C₅₀ carotenoid γ-cyclase activity and which has an amino acid sequence as set forth in all or part of SEQ ID NO: 15 or an amino acid sequence which is at least 90% identical to SEQ ID NO: 15, wherein said amino acid sequence comprises one or more of the following:
(a) a glycine residue at position 100;
(b) a glycine residue at position 103;
(c) a proline residue at position 107;
or a nucleotide sequence which is the complement of any aforesaid sequence, wherein the position numbers are stated with reference to SEQ ID NO. 15, preferably wherein the nucleic acid molecule comprises a nucleotide sequence as set forth in SEQ ID NO: 14 or a part of variant thereof, or a complement thereof.

18. The method of claim 1 comprising the introduction of a further nucleic acid molecule into said host cell, wherein said nucleic acid molecule encodes an enzyme capable of glycosylating sarcinxanthin.

19. The method of claim 18, wherein said further nucleic acid molecule encodes crtX from M. luteus or a functional equivalent thereof, preferably wherein the nucleic acid comprises:
(i) a nucleotide sequence as set forth in all or part of SEQ ID NO: 33 or 16, or which is degenerate therewith, or a nucleotide sequence with at least 70% sequence identity to SEQ ID NO: 33 or 16;
(ii) a nucleotide sequence which hybridizes to SEQ ID NO: 33 or 16 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 34 or 17 or which comprises an amino acid sequence which is at least 70% identical to SEQ ID NO: 34 or 17.

20. The method of claim 19, wherein said further nucleic acid molecule comprises a nucleotide sequence encoding a protein having sarcinaxanthin glycosylase activity and an amino acid sequence as set forth in all or part of SEQ ID NO: 34 or an amino acid sequence which is at least 90% identical to SEQ ID NO: 34, wherein said amino acid sequence comprises one or more of the following:
(a) histidine at position 62;
(b) serine at position 109;
(c) arginine at position 129;
(d) alanine at position 138;
(e) arginine at position 248;
(f) proline at position 251;
or a nucleotide sequence which is the complement of any aforesaid sequence, wherein the position numbers are stated with reference to SEQ ID NO. 34, preferably wherein the nucleic acid molecule comprises a nucleotide sequence as set forth in SEQ ID NO: 33 or a part of variant thereof, or a complement thereof.

21. The method of claim 1, wherein the expression of one or more said nucleic acid molecules is inducible.

22. The method of claim 1, wherein said host cell is a microorganism particularly a bacterium.

23. The method of claim 22, wherein said bacterium is selected from Escherichia sp., Salmonella, Klebsiella, Proteus, Yersinia, Azotobacter sp., Pseudomonas sp., Xanthomonas sp., Agrobacterium sp., Alcaligenes sp., Bordatella sp., Haemophilus influenzae, Methylophilus methylotrophus, Rhizobium sp., Thiobacillus sp. and Clavibacter sp., preferably wherein the host cell is an Escherichia coli cell or a Corynebacterium glutamicum cell.

24. An isolated nucleic acid molecule comprising or consisting of all or a part of a nucleotide sequence as set forth in SEQ ID NO: 37 or which has at least 90% sequence identity to SEQ ID NO. 37, which molecule encodes one or more proteins having activity in the biosynthesis of sarcinaxanthin, and wherein any nucleic acid molecule which comprises a nucleotide sequence which is a part of SEQ ID NO. 37 or which is at least 90% identical to SEQ ID NO. 37 encodes proteins which are able to synthesise sarcinaxanthin at substantially the same level as the proteins encoded by SEQ ID NO: 37 when expressed in a host cell.

25. The nucleic acid molecule of claim 24, wherein said part of said nucleic acid molecule comprises or consists of all or a part of a nucleotide sequence as set forth in SEQ ID NO: 26 or which has at least 90% sequence identity to SEQ ID NO. 26, which molecule encodes one or more proteins having activity in the biosynthesis of sarcinaxanthin, and wherein any nucleic acid molecule which comprises a nucleotide sequence which is a part of SEQ ID NO. 26 or which is at least 90% identical to SEQ ID NO. 26 encodes proteins which are able to synthesise sarcinaxanthin at substantially the same level as the proteins encoded by SEQ ID NO: 26 when expressed in a host cell.

26. The nucleic acid molecule of claim 24, wherein said part of said nucleic acid molecule comprises a nucleotide sequence encoding all or part of a protein having an amino acid sequence as set forth in SEQ ID NO: 11 or an amino acid sequence which is at least 90% identical to SEQ ID NO: 11 and wherein said nucleotide sequence encodes a lycopene elongase with a lycopene to flavuxanthin conversion efficiency of at least 30%, when expressed in a host cell, or a nucleic acid molecule which comprises a nucleotide sequence which is the complement of any aforesaid sequence.

27. The nucleic acid molecule of claim 26, wherein said part of said nucleic acid molecule comprises:
(i) a nucleotide sequence as set forth in SEQ ID NO: 10;
(ii) a nucleotide sequence which is degenerate with the sequence of SEQ ID NO: 10;
(iii) a nucleotide sequence which has at least 90% sequence identity to SEQ ID NO: 10;
(iv) a nucleotide sequence which is a part of the nucleotide sequence of SEQ ID NO: 10 or of a nucleotide sequence which is degenerate therewith; or
(v) a nucleotide sequence which is complementary to any of (i) to (iv) above.

28. The nucleic acid molecule of claim 24, wherein said part of said nucleic acid molecule comprises a nucleotide sequence encoding all or part of a protein having an amino acid sequence selected from the sequences as set forth in any one of SEQ ID NO: 11, 13 and 15 or an amino acid sequence which has at least 90% sequence identity to SEQ ID NO: 11, 13 or 15, and wherein said nucleotide sequence encodes a protein which when expressed in a lycopene-producing host cell together with each of the other said proteins results in at least 91% of the total carotenoids produced being sarcinaxanthin, or a nucleic acid molecule which comprises a nucleotide sequence which is the complement of any aforesaid sequence.

29. The nucleic acid molecule of claim 24, wherein said part of said nucleic acid molecule comprises a nucleotide sequence encoding all or part of a protein having an amino acid sequence selected from the sequences as set forth in any one of SEQ ID NO: 11, 13 and 15 or an amino acid sequence which has at least 90% sequence identity to SEQ ID NO: 11, 13 or 15, wherein said nucleotide sequence encodes a protein which when expressed in a lycopene-producing host cell together with each of the other said proteins results in sarcinaxanthin production to a level of at least 150 μg/g of cell dry weight (CDW).

30. The nucleic acid molecule of claim 28, wherein said nucleic acid molecule comprises:
(i) a nucleotide sequence selected from sequences as set forth in SEQ ID NO: 10, 12 and 14;
(ii) a nucleotide sequence which is degenerate with the sequence of any one of SEQ ID NOs: 10, 12 or 14;
(iii) a nucleotide sequence which has at least 90% sequence identity to any one of SEQ ID NOs: 10, 12 or 14;
(iv) a nucleotide sequence which is a part of the nucleotide sequence of any one of SEQ ID NOs: 10, 12 or 14 or of a nucleotide sequence which is degenerate therewith; or
(v) a nucleotide sequence which is complementary to any of (i) to (iv) above.

31. The nucleic acid molecule of claim 30, wherein said nucleic acid molecule comprises a nucleotide sequence encoding a protein having lycopene elongase activity and an amino acid sequence as set forth in all or part of SEQ ID NO: 11 or an amino acid sequence which is at least 90% identical to SEQ ID NO: 11, wherein said amino acid sequence comprises one or more of the following:
(a) alanine at position 8;
(b) valine at position 88;
(c) valine at position 158;
or a nucleotide sequence which is the complement of any aforesaid sequence, wherein the position numbers are stated with reference to SEQ ID NO. 11, preferably wherein the nucleic acid molecule comprises a nucleotide sequence as set forth in SEQ ID NO: 10 or a part of variant thereof, or a complement thereof.

32. The nucleic acid molecule of claim 30, wherein said nucleic acid molecule comprises a nucleotide sequence encoding a protein which contributes to C₅₀ carotenoid γ-cyclase activity and which has an amino acid sequence as set forth in all or part of SEQ ID NO: 13 or an amino acid sequence which is at least 90% identical to SEQ ID NO: 13, wherein said amino acid sequence comprises one or more of the following:
(a) valine at position 44;
(b) valine at position 64;
(c) glycine at position 103;
(d) arginine at position 104;
(e) proline at position 111;
(f) glycine at position 117;
or a nucleotide sequence which is the complement of any aforesaid sequence, wherein the position numbers are stated with reference to SEQ ID NO. 13, preferably wherein the nucleic acid molecule comprises a nucleotide sequence as set forth in SEQ ID NO: 12 or a part of variant thereof, or a complement thereof.

33. The nucleic acid molecule of claim 30, wherein said nucleic acid molecule comprises a nucleotide sequence encoding a protein which contributes to C₅₀ carotenoid γ-cyclase activity and which has an amino acid sequence as set forth in all or part of SEQ ID NO: 15 or an amino acid sequence which is at least 90% identical to SEQ ID NO: 15, wherein said amino acid sequence comprises one or more of the following:
(a) a glycine residue at position 100;
(b) a glycine residue at position 103;
(c) a proline residue at position 107;
or a nucleotide sequence which is the complement of any aforesaid sequence, wherein the position numbers are stated with reference to SEQ ID NO. 15, preferably wherein the nucleic acid molecule comprises a nucleotide sequence as set forth in SEQ ID NO: 14 or a part of variant thereof, or a complement thereof.

34. The nucleic acid molecule of claim 24, wherein said part of said nucleic acid molecule comprises a nucleotide sequence encoding all or part of a protein having an amino acid sequence as set forth in SEQ ID NO: 34 or an amino acid sequence which is at least 90% identical to SEQ ID NO: 34 and wherein said nucleotide sequence encodes a sarcinaxanthin glycosylase enzyme, which activity results in the production of both sarcinaxanthin mono- and diglucosides, when expressed in a host cell, or a nucleic acid molecule which comprises a nucleotide sequence which is the complement of any aforesaid sequence.

35. The nucleic acid molecule of claim 34, wherein said part of said nucleic acid molecule comprises:
(i) a nucleotide sequence as set forth in SEQ ID NO: 33;
(ii) a nucleotide sequence which is degenerate with the sequence of SEQ ID NO: 33,
(iii) a nucleotide sequence which has at least 90% sequence identity to SEQ ID NO: 33;
(iv) a nucleotide sequence which is a part of the nucleotide sequence of SEQ ID NO: 33 or of a nucleotide sequence which is degenerate therewith; or
(v) a nucleotide sequence which is complementary to any of (i) to (iv) above.

36. The nucleic acid molecule of claim 35, wherein said nucleic acid molecule comprises a nucleotide sequence encoding a protein having sarcinaxanthin glycosylase activity and an amino acid sequence as set forth in all or part of SEQ ID NO: 34 or an amino acid sequence which is at least 90% identical to SEQ ID NO: 34, wherein said amino acid sequence comprises one or more of the following:
(a) histidine at position 62;
(b) serine at position 109;
(c) arginine at position 129;
(d) alanine at position 138;
(e) arginine at position 248;
(f) proline at position 251;
or a nucleotide sequence which is the complement of any aforesaid sequence, wherein the position numbers are stated with reference to SEQ ID NO. 34, preferably wherein the nucleic acid molecule comprises a nucleotide sequence as set forth in SEQ ID NO: 33 or a part of variant thereof, or a complement thereof.

37. A vector comprising the isolated nucleic acid molecule of claim 24.

38. An isolated protein encoded by the nucleic acid molecule of claim 24.

39. A strain of Micrococcus luteus as deposited under number DSM 23579 at the DSMZ, or a mutant or modified strain thereof which produces sarcinaxanthin or a derivative thereof.


SEQUENCE LISTING (51 sequences)

SEQ ID NO: 1 - DNA, 7163 bp, Micrococcus luteus NCTC2665
gcggagtcctcgtccgcctcggcgtcgtcgctgtccgcggccccggccgactacgaggccggcacgtgcttcaccgccccgctcggcgcgcgtgacctgtcctccttcgagaccaccgactgcgagggcgcccacaccgcggagtacctgtgggccgtgccggccgtggccgagggtgaggaggccgaccccgccgccgcccagacctgcaccgcccaggcccagcgcctgagcgaggagaaggaggaccagctgaacggggccgtcctgacctcctccgagctgggcaactacggcaccgacgagaagcactgcgtcgtgtacggggtctccggtgagtgggagggtcagatcgtggacccggagatcaccctggagacggcgtccgccgacgcctgatcccgccggcggccccgtgcgtcgtgagatcgcgccgcccgggaccgccgcggatggacgcgggaccggcgcggcccgtagtgtcttctgcgtccagaagttagacggtcgaacaggtgcggcggtcggtgccgcgtcgtgtccgccaccgaggaggcgccatgggtgaagcgaggacgggcggcgaggccgcgctctccggggtgaccgccgagctggacgccgcgctccgacacgccgcggcccaggcgcccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgactgcggccggcgagctcgaggacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgccgcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctgagatggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggccgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtccctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtccgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtcgcggcccgcttccaggacccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtggaggtcgcggaccgtcccgcccccgccggccgcctcggacgcctggccgcccgcctgcccaggccgggagcagcccgcggggacgagggccgacgtcgccgcccgggccgggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggggaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggccagggctggcgggtggaccgcgccggggacgcccaggtggaggccgtggcggaccgggccctcgatcagctggcccgctgggccgggatccccgacctggccgagcgcatcgtggtgcggcgcacctacgggcccggtgacttcgccgcggacgtgcacgcctggcggggttcgctgctgggccccgggcacacgctggcgcagtcggccatgttccgcccctcggtgcgggacgcggacgtggccggcctgatgtacgcgggctcctcggtgcgcccgggaatcggggtgcccatgtgcctgatctccgccgaagtggtccgggacgaactgcgccacgacgcgcgcagggcccggcccgcgggccccggggggagcggcacatgatccgcaccctcttctgggtgtcccggccggtcagctgggtgaacacggcctacccgttcgccgccgccgcgatcctgaccggggggctgcccgcgtggctggtggtcctgggcgtcgtgttcttcctggtgccctacaacctggccatgtacggcatcaatgacgtgttcgacttcgcctcggacctgcgcaacccccgcaaggggggtgtggagggctccgtgctgggcgaccccgcggtgcgccgccgggtgctggcgtggtcggtgctgctgcccgtgccgttcgtggccgtgctcgcgggctggtccgccgtgcggggcgagtgggccgccgtgctggtgctcgcggtgagcctgttcgcggtggtggcgtactcctgggcggggctgcggttcaaggagcggcccttcctggacgccgccacctccgccacccacttcgtctcccccgcggtctacggcctcgcgctggccggggcgacccccacgcccgccctggcggcgctgctgggggcgttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccgctcgcggcgctgctggccgtgccctacgtggtgaacaccctgcgcttccgccgcatcacggacgccacctcgggcgcggcccaccgcggctggcagctgttccttccgctgaactacgtgaccggcttcctcgtgaccctgctgctgatcgggtgggcgctgacccggggggcggcggcatgatctacctgctggccctgctgggtgtcatcggctgcatgctgctggtggaccggcgcttcgagctgttcctgtggcatcgcccgctcccggcgctgctggtgctggccgccggggtggcctacttcttcgcctgggacctgtgggggatcgccgaaggcgtgttcctgcaccggcagtcgccctacatgaccggggtgatgctcgccccccagctgcccctggaggaggggttcttcctgctcttcctcagccagatcacgatggtgctgttcaccggggcgctgcgcctgctgcgcggccggcgaggtgacgcccgtgccgcgacggcggccgatccgaccgaccgggggagccggtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtcgtgccgatcggcgcggccgtcggccgggacgccgcccccacccggacgatcgctgacatgctgccgttgatccccgcagacctgctgcgcgcgctcggcctgatcctcgtcccggtcgcggcggtgcacgccggatggccgtccgcggcggcgatgctgctcgtgttcggctcccagtggctcacccgctggctcgccccgggcggcgccctggactgggccgcgcaggcggtcctgctgctggccgggtggctgagcgtcatcggcctctacccgcgggtgccgtggctggacctgctcgtgcacgccgccgcctccgccgtggtcgcctgtctgacggcactggtggtgggggcgtggctccggcgtcgggggaccgaggccgggcaggccgtggcgctgctcggcccgggcctggccgggctggggatcgcggccgccgccgtggccctgggcgtggtgtgggagctggccgaatggtgggggcacacggcggtgaccccggagatcggcgtgggctacacggacaccatcggcgacctcgccgccgatctcgtcggcgccggggtcggcgccgccctcgccgtgtgccgggggcgcacccggtgaccccggcccgccccacggtctccgtggtcgtcccggtgctcgacgacgccgagcacctgcgcgtgtgcctcgcgctgctggccgcccagagccggccggcgctggaggtggtggtggtggacaacggctgcgtggacgactcggcggtgctcgcccgcgccgccggcgcgcgggtggtgcgcgagccgcgccgcggggtcccggccgcggcggccgccggcctggacgccgcggtcggggagctgctggtgcgctgcgacgccgacacgcggatgcccgcggactggctcgaacggatcgtggcccggttcgacgccgaccccgggctcgacgccctcaccgggccggggaccttccacgaccagcccggcctccggggacaggtgcgggcggcgctctacaccggcacgtaccgctggggggcgggcgccgcggtggcggccacccccgtctggggctccaactgcgccctgcgcgccgaggcgtggcaggctgtgcggacccgcgtccaccgcgaacgcggggacgtgcacgatgacctggacctgtccttccagctggccctggccggccgccggatccggttcgatccggacctgcgggtggaggtcgccgggcgcatcttccactccctgcgccagcgggtgcggcagggccggatggcggtcaccaccctgcaggtcaactgggcccgactgtcccccgggcggcgttggctgcgccgggcggcccgggcacacccccggtcccgctgggggcgtggccccgacggtcagtcccgggactga

SEQ ID NO: 2 - DNA, 363 bp, Micrococcus luteus NCTC2665
atgatctacctgctggccctgctgggtgtcatcggctgcatgctgctggtggaccggcgcttcgagctgttcctgtggcatcgcccgctcccggcgctgctggtgctggccgccggggtggcctacttcttcgcctgggacctgtgggggatcgccgaaggcgtgttcctgcaccggcagtcgccctacatgaccggggtgatgctcgccccccagctgcccctggaggaggggttcttcctgctcttcctcagccagatcacgatggtgctgttcaccggggcgctgcgcctgctgcgcggccggcgaggtgacgcccgtgccgcgacggcggccgatccgaccgaccgggggagccggtga

SEQ ID NO: 3 - protein, 120 aa, Micrococcus luteus NCTC2665
MIYLLALLGVIGCMLLVDRRFELFLWHRPLPALLVLAAGVAYFFAWDLWGIAEGVFLHRQSPYMTGVMLAPQLPLEEGFFLLFLSQITMVLFTGALRLLRGRRGDARAATAADPTDRGSR

SEQ ID NO: 4 - DNA, 354 bp, Micrococcus luteus NCTC2665
gtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtcgtgccgatcggcgcggccgtcggccgggacgccgcccccacccggacgatcgctga

SEQ ID NO: 5 - protein, 117 aa, Micrococcus luteus NCTC2665
VTFLDLVLVFVGFALAVLVGAALVGRVRGEHLRAVAATLVALWALTAVFDNVMIAAGLFDYGHELLVGAYVGQAPVEDFAYPLGSALLLPALWLLLTSRRADRRGRRPGRRPHPDDR

SEQ ID NO: 6 - DNA, 885 bp, Micrococcus luteus NCTC2665
atgatccgcaccctcttctgggtgtcccggccggtcagctgggtgaacacggcctacccgttcgccgccgccgcgatcctgaccggggggctgcccgcgtggctggtggtcctgggcgtcgtgttcttcctggtgccctacaacctggccatgtacggcatcaatgacgtgttcgacttcgcctcggacctgcgcaacccccgcaaggggggtgtggagggctccgtgctgggcgaccccgcggtgcgccgccgggtgctggcgtggtcggtgctgctgcccgtgccgttcgtggccgtgctcgcgggctggtccgccgtgcggggcgagtgggccgccgtgctggtgctcgcggtgagcctgttcgcggtggtggcgtactcctgggcggggctgcggttcaaggagcggcccttcctggacgccgccacctccgccacccacttcgtctcccccgcggtctacggcctcgcgctggccggggcgacccccacgcccgccctggcggcgctgctgggggcgttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccgctcgcggcgctgctggccgtgccctacgtggtgaacaccctgcgcttccgccgcatcacggacgccacctcgggcgcggcccaccgcggctggcagctgttccttccgctgaactacgtgaccggcttcctcgtgaccctgctgctgatcgggtgggcgctgacccggggggcggcggcatga

SEQ ID NO: 7 - DNA, 864 bp, Corynebacterium glutamicum
atgatggaaaaaataagactgattctattgtcatctcgccccattagctgggtcaataccgcctacccttttgggctggcatacctattaaatgcaggagagattgactggctgttttggctaggcatcgtgttttttcttatcccgtataacatcgccatgtatggcatcaacgatgtttttgattacgaatctgacatacgtaatccccgcaaaggcggcgtcgagggggccgtgctcccgaaaagttcccacagcacactgttatgggcatcggctatctcaacaattcctttcctagttattcttttcatatttggcacctggatgtcgtctttatggctgacaatctcagtgctagcagtgattgcttattcagcaccgaaattgcgttttaaagaacgcccctttatcgatgctctaacatcttctactcacttcacttcacctgcattaatcggtgcaacgatcactggaacatctccttcagcagcgatgtggatagcactgggatcctttttcttgtggggcatggccagtcagatccttggagcagtacaggatgttaatgcagaccgggaagctaatctgagctcaattgccactgtaattggggcgcgtggagccattcggctatcagtagtactttatttactagctgctgttttagtcactactttgcctaatccggcgtggatcatcgggattgcgattctaacttacgtatttgatgccgcacgattttggaacattacagatgccagttgtgaacaggctaatcgcagttggaaagttttcctgtggctgaactactttgttggtgctgtgataacgatactgttaatagcaattcatcagatataa

SEQ ID NO: 8 - protein, 294 aa, Micrococcus luteus NCTC2665
MIRTLFWVSRPVSWVNTAYPFAAAAILTGGLPAWLVVLGVVFFLVPYNLAMYGINDVFDFASDLRNPRKGGVEGSVLGDPAVRRRVLAWSVLLPVPFVAVLAGWSAVRGEWAAVLVLAVSLFAVVAYSWAGLRFKERPFLDAATSATHFVSPAVYGLALAGATPTPALAALLGAFFLWGMASQMFGAVQDVVPDREGGLASVATVLGARRTVLLAAGLYAAAGLLLLATDPPGPLAALLAVPYVVNTLRFRRITDATSGAAHRGWQLFLPLNYVTGFLVTLLLIGWALTRGAAA

SEQ ID NO: 9 - protein, 287 aa, Corynebacterium glutamicum
MMEKIRLILLSSRPISWVNTAYPFGLAYLLNAGEIDWLFWLGIVFFLIPYNIAMYGINDVFDYESDIRNPRKGGVEGAVLPKSSHSTLLWASAISTIPFLVILFIFGTWMSSLWLTISVLAVIAYSAPKLRFKERPFIDALTSSTHFTSPALIGATITGTSPSAAMWIALGSFFLWGMASQILGAVQDVNADREANLSSIATVIGARGAIRLSVVLYLLAAVLVTTLPNPAWIIGIAILTYVFDAARFWNITDASCEQANRSWKVFLWLNYFVGAVITILLIAIHQI

SEQ ID NO: 10 - DNA, 885 bp, Micrococcus luteus Otnes 7
atgatccgcaccctcttctgggcgtcccggccggtcagctgggtgaacacggcgtacccgttcgccgccgccgcgatcctgaccggggggctgcccgcgtggctggtggtcctgggcgtcgtgttcttcctcgtgccctacaacctggccatgtacggcatcaatgacgtgttcgacttcgcctcggacctgcgcaacccccgcaaggggggcgtggagggctccgtgctgggcgaccccgcggtgcgccgccgggtgctggtgtggtcggtgctgctgcccgtcccgttcgtggccgtgctcgcgggctggtccgccgtgcggggcgagtgggccgccgtgctggtgctggcggtgagcctgttcgcggtggtggcgtactcctgggcggggctgcggttcaaggagcggcccttcctggacgccgcgacctccgccacccacttcgtctcccccgcggtctacggcctcgtgctggccggggcgacccccacgcccgccctggcggcgctgctgggggccttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccccttgcggcgctgctggccgtgccctacgtggtgaacaccctgcgcttccgccgcatcacggacgccacctcgggcgcggcccaccgcggctggcagctgttcctccccctgaactacgtgaccggcttcctcgtgaccctgctgctgatcgggtgggcgctgacccggggggcggcggcatga

SEQ ID NO: 11 - protein, 294 aa, Micrococcus luteus Otnes 7
MIRTLFWASRPVSWVNTAYPFAAAAILTGGLPAWLVVLGVVFFLVPYNLAMYGINDVFDFASDLRNPRKGGVEGSVLGDPAVRRRVLVWSVLLPVPFVAVLAGWSAVRGEWAAVLVLAVSLFAVVAYSWAGLRFKERPFLDAATSATHFVSPAVYGLVLAGATPTPALAALLGAFFLWGMASQMFGAVQDVVPDREGGLASVATVLGARRTVLLAAGLYAAAGLLLLATDPPGPLAALLAVPYVVNTLRFRRITDATSGAAHRGWQLFLPLNYVTGFLVTLLLIGWALTRGAAA

SEQ ID NO: 12 - DNA, 363 bp, Micrococcus luteus Otnes 7
atgatctacctgctggccctgctgggtgtcatcggctgcatgctgctggtggaccggcgcttcgagctgttcctgtggcatcgcccgctcccggcgctgctggtgctggccgccggggtggcctacttcgtcgcctgggacctgtgggggatcgccgaaggcgtgttcctgcaccggcagtcgccctacgtgaccggggtgatgctcgccccccagctgcccctggaggaggggttcttcctgctcttcctcagccagatcacgatggtgctgttcaccggggcgctgcgcctgctgcgcggccggggacgcgacgcccgtgccgcgacgccggccgatccgaccgacggggggagccggtga

SEQ ID NO: 13 - protein, 120 aa, Micrococcus luteus Otnes 7
MIYLLALLGVIGCMLLVDRRFELFLWHRPLPALLVLAAGVAYFVAWDLWGIAEGVFLHRQSPYVTGVMLAPQLPLEEGFFLLFLSQITMVLFTGALRLLRGRGRDARAATPADPTDGGSR

SEQ ID NO: 14 - DNA, 356 bp, Micrococcus luteus Otnes 7
gtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtggtcgtgccggtcggcgcggccctcggccgggacgccgcccccacccggacgatcgctg

SEQ ID NO: 15 - protein, 118 aa, Micrococcus luteus Otnes 7
VTFLDLVLVFVGFALAVLVGAALVGRVRGEHLRAVAATLVALWALTAVFDNVMIAAGLFDYGHELLVGAYVGQAPVEDFAYPLGSALLLPALWLLLTSRGRAGRRGPRPGRRPHPDDR

SEQ ID NO: 16 - DNA, 792 bp, Micrococcus luteus NCTC2665
gtgaccccggcccgccccacggtctccgtggtcgtcccggtgctcgacgacgccgagcacctgcgcgtgtgcctcgcgctgctggccgcccagagccggccggcgctggaggtggtggtggtggacaacggctgcgtggacgactcggcggtgctcgcccgcgccgccggcgcgcgggtggtgcgcgagccgcgccgcggggtcccggccgcggcggccgccggcctggacgccgcggtcggggagctgctggtgcgctgcgacgccgacacgcggatgcccgcggactggctcgaacggatcgtggcccggttcgacgccgaccccgggctcgacgccctcaccgggccggggaccttccacgaccagcccggcctccggggacaggtgcgggcggcgctctacaccggcacgtaccgctggggggcgggcgccgcggtggcggccacccccgtctggggctccaactgcgccctgcgcgccgaggcgtggcaggctgtgcggacccgcgtccaccgcgaacgcggggacgtgcacgatgacctggacctgtccttccagctggccctggccggccgccggatccggttcgatccggacctgcgggtggaggtcgccgggcgcatcttccactccctgcgccagcgggtgcggcagggccggatggcggtcaccaccctgcaggtcaactgggcccgactgtcccccgggcggcgttggctgcgccgggcggcccgggcacacccccggtcccgctgggggcgtggccccgacggtcagtcccgggactga

SEQ ID NO: 17 - protein, 263 aa, Micrococcus luteus NCTC2665
VTPARPTVSVVVPVLDDAEHLRVCLALLAAQSRPALEVVVVDNGCVDDSAVLARAAGARVVREPRRGVPAAAAAGLDAAVGELLVRCDADTRMPADWLERIVARFDADPGLDALTGPGTFHDQPGLRGQVRAALYTGTYRWGAGAAVAATPVWGSNCALRAEAWQAVRTRVHRERGDVHDDLDLSFQLALAGRRIRFDPDLRVEVAGRIFHSLRQRVRQGRMAVTTLQVNWARLSPGRRWLRRAARAHPRSRWGRGPDGQSRD

SEQ ID NO: 18 - DNA, 1077 bp, Micrococcus luteus NCTC2665
atgggtgaagcgaggacgggcggcgaggccgcgctctccggggtgaccgccgagctggacgccgcgctccgacacgccgcggcccaggcgcccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgactgcggccggcgagctcgaggacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgccgcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctga

SEQ ID NO: 19 - protein, 358 aa, Micrococcus luteus NCTC2665
MGEARTGGEAALSGVTAELDAALRHAAAQAPGSAAFAELLDSLHVHVGAGKLIRPRLVELGWRLATADPVPPSGRAAVDRLGAAFELLHTALLVHDDVIDRDVLRRGQPAVHASARHRLEARGVPAADAAHAGVAVALIAGDVLLTQAFRLAATCAADTARAAEAAAVVFDAAAVTAAGELEDVLLGLSRHTGEEPDPDRILAMQRLKTAHYTVGAPLRAGALLAGADPDLARAMGEAGADLGAAYQVIDDVLGVFGDPGETGKSADGDLREGKATVLTAHGRRIPAVRALLDAGPATPADIEAARRALEAAGAREHALDVAAELTVRARERIAALPLDETVRAEFADACHAVLTRRS

SEQ ID NO: 20 - DNA, 897 bp, Micrococcus luteus NCTC2665
atggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggccgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtccctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatga

SEQ ID NO: 21 - protein, 298 aa, Micrococcus luteus NCTC2665
MAAPTPSPAALYTRTAHTAAAQVIRRYSTSFSWACRTLPRQARQDVATIYAMVRVADEVVDGVAVAAGLDEAGVRAALDDYERACEAAMASGFATDPVLHAFADVARRHGITPELTRPFFASMRADLGIREHGAESLDAYIHGSAEVVGLMCLQVFLSLPGTRARTPGQRQELRAQASRLGAAFQKVNFLRDLAADHHELGRTYLPGAAPGVLTEARKAELVAEVRADLDAALPGIRVLDPGAGRAVALAHGLFAALVDRIEATPAAELAHRRVRVPDHQKARIAARVLARGRRGGRR

SEQ ID NO: 22 - DNA, 1701 bp, Micrococcus luteus NCTC2665
atgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtccgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtcgcggcccgcttccaggacccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtggaggtcgcggaccgtcccgcccccgccggccgcctcggacgcctggccgcccgcctgcccaggccgggagcagcccgcggggacgagggccgacgtcgccgcccgggccgggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggggaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggccagggctggcgggtggaccgcgccggggacgcccaggtggaggccgtggcggaccgggccctcgatcagctggcccgctgggccgggatccccgacctggccgagcgcatcgtggtgcggcgcacctacgggcccggtgacttcgccgcggacgtgcacgcctggcggggttcgctgctgggccccgggcacacgctggcgcagtcggccatgttccgcccctcggtgcgggacgcggacgtggccggcctgatgtacgcgggctcctcggtgcgcccgggaatcggggtgcccatgtgcctgatctccgccgaagtggtccgggacgaactgcgccacgacgcgcgcagggcccggcccgcgggccccggggggagcggcacatga

SEQ ID NO: 23 - protein, 566 aa, Micrococcus luteus NCTC2665
MSARDTALGPRTVVVGGGFAGLATAGLLARDGHRVTLLERGAVLGGRAGRWSEAGFTFDTGPSWYLMPEVIDRWFRLMGTSAAERLDLRRLDPGYRVYFEGHLHEPPVDVRTGHAETLFESLEPGAGRRLRAYLDSASRIYGLAKEHFLYTDFRRPAALAHPDVLRALPALGPQLLGGLRSHVAARFQDPRLRQILGYPAVFLGTSPDRAPAMYHLMSHLDLADGVQYPLGGFAALVDAMAEVVREAGVEIRTGVEATAVEVADRPAPAGRLGRLAARLPRPGAARGDEGRRRRPGRVTGVAWRSDDGAAGRLDADVVVAAADLHHVQTRLLPPGRRVAESTWDRRDPGPSGVLVCVGVRGSLPQLAHHTLLFTADWEDNFGRIERGEDLAADTSIYVSRTSATDPGVAPEGDENLFILVPAPAEPGWGRGGIRVRDGQGWRVDRAGDAQVEAVADRALDQLARWAGIPDLAERIVVRRTYGPGDFAADVHAWRGSLLGPGHTLAQSAMFRPSVRDADVAGLMYAGSSVRPGIGVPMCLISAEVVRDELRHDARRARPAGPGGSGT

SEQ ID NO: 24 - DNA, 609 bp, Micrococcus luteus NCTC2665
gtgccgatcggcgcggccgtcggccgggacgccgcccccacccggacgatcgctgacatgctgccgttgatccccgcagacctgctgcgcgcgctcggcctgatcctcgtcccggtcgcggcggtgcacgccggatggccgtccgcggcggcgatgctgctcgtgttcggctcccagtggctcacccgctggctcgccccgggcggcgccctggactgggccgcgcaggcggtcctgctgctggccgggtggctgagcgtcatcggcctctacccgcgggtgccgtggctggacctgctcgtgcacgccgccgcctccgccgtggtcgcctgtctgacggcactggtggtgggggcgtggctccggcgtcgggggaccgaggccgggcaggccgtggcgctgctcggcccgggcctggccgggctggggatcgcggccgccgccgtggccctgggcgtggtgtgggagctggccgaatggtgggggcacacggcggtgaccccggagatcggcgtgggctacacggacaccatcggcgacctcgccgccgatctcgtcggcgccggggtcggcgccgccctcgccgtgtgccgggggcgcacccggtga

SEQ ID NO: 25 - protein, 202 aa, Micrococcus luteus NCTC2665
VPIGAAVGRDAAPTRTIADMLPLIPADLLRALGLILVPVAAVHAGWPSAAAMLLVFGSQWLTRWLAPGGALDWAAQAVLLLAGWLSVIGLYPRVPWLDLLVHAAASAVVACLTALVVGAWLRRRGTEAGQAVALLGPGLAGLGIAAAAVALGVVWELAEWWGHTAVTPEIGVGYTDTIGDLAADLVGAGVGAALAVCRGRTR

SEQ ID NO: 26 - DNA, 5379 bp, Micrococcus luteus Otnes 7
atgggtgaagcgaggacgggcggcgaggccgcgctctccggggtgaccgccgagctggacgccgcgctccgacatgccgcggcccaggcacccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgaccgcggccggcgagctcgaagacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgcctcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctgagatggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggctgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtcgctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtctgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtggcggcccgcttccaggatccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtcgaggtggtggaccgtcccgcccccgccggccgcctcggacgcctggccgcccgcctgcccaggccgggagcagcccgcggggacgagggccgacgtcgccgcccgggccaggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggagaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggcgagggctggcgggtggaccgcgccggggacgcccaggtggaggccgtggcggaccgggccctcgaccagctggcccgctgggccgggatcccggacctggccgagcgcatcgtggtgcggcgcacctacgggcccggtgacttcgccgcggacgtgcacgcctggcggggttcgctgctgggccccgggcacacgctggcgcagtcggccatgttccgtccctcggtgcgggacgcggacgtggccggcctgatgtacgcgggctcctcggtgcgcccgggcatcggggtgcccatgtgtctgatctccgccgaagtggtccgggacgaactgcgccacgacgcgcgcagggcccggcccgcgggccccggggggagcggcacatgatccgcaccctcttctgggcgtcccggccggtcagctgggtgaacacggcgtacccgttcgccgccgccgcgatcctgaccggggggctgcccgcgtggctggtggtcctgggcgtcgtgttcttcctcgtgccctacaacctggccatgtacggcatcaatgacgtgttcgacttcgcctcggacctgcgcaacccccgcaaggggggcgtggagggctccgtgctgggcgaccccgcggtgcgccgccgggtgctggtgtggtcggtgctgctgcccgtcccgttcgtggccgtgctcgcgggctggtccgccgtgcggggcgagtgggccgccgtgctggtgctggcggtgagcctgttcgcggtggtggcgtactcctgggcggggctgcggttcaaggagcggcccttcctggacgccgcgacctccgccacccacttcgtctcccccgcggtctacggcctcgtgctggccggggcgacccccacgcccgccctggcggcgctgctgggggccttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccccttgcggcgctgctggccgtgccctacgtggtgaacaccctgcgcttccgccgcatcacggacgccacctcgggcgcggcccaccgcggctggcagctgttcctccccctgaactacgtgaccggcttcctcgtgaccctgctgctgatcgggtgggcgctgacccggggggcggcggcatgatctacctgctggccctgctgggtgtcatcggctgcatgctgctggtggaccggcgcttcgagctgttcctgtggcatcgcccgctcccggcgctgctggtgctggccgccggggtggcctacttcgtcgcctgggacctgtgggggatcgccgaaggcgtgttcctgcaccggcagtcgccctacgtgaccggggtgatgctcgccccccagctgcccctggaggaggggttcttcctgctcttcctcagccagatcacgatggtgctgttcaccggggcgctgcgcctgctgcgcggccggggacgcgacgcccgtgccgcgacgccggccgatccgaccgacggggggagccggtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtggtcgtgccggtcggcgcggccctcggccgggacgccgcccccacccggacgatcgctgagcggccgcaaaaaaatcactagtgcggccgcctgcaggtcgaccatatgggagagctcccaacgcgttggatgcatagcttgagtattctatagtgtcacctaaatagctggcg

SEQ ID NO: 27 - DNA, 1077 bp, Micrococcus luteus Otnes 7
atgggtgaagcgaggacgggcggcgaggccgcgctctccggggtgaccgccgagctggacgccgcgctccgacatgccgcggcccaggcacccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgaccgcggccggcgagctcgaagacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgcctcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctga

SEQ ID NO: 28 - protein, 358 aa, Micrococcus luteus Otnes 7
MGEARTGGEAALSGVTAELDAALRHAAAQAPGSAAFAELLDSLHVHVGAGKLIRPRLVELGWRLATADPVPPSGRAAVDRLGAAFELLHTALLVHDDVIDRDVLRRGQPAVHASARHRLEARGVPAADAAHAGVAVALIAGDVLLTQAFRLAATCAADTARAAEAAAVVFDAAAVTAAGELEDVLLGLSRHTGEEPDPDRILAMQRLKTAHYTVGAPLRAGALLAGADPDLARAMGEAGADLGAAYQVIDDVLGVFGDPGETGKSADGDLREGKATVLTAHGRLIPAVRALLDAGPATPADIEAARRALEAAGAREHALDVAAELTVRARERIAALPLDETVRAEFADACHAVLTRRS

SEQ ID NO: 29 - DNA, 897 bp, Micrococcus luteus Otnes 7
atggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggctgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtcgctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatga

SEQ ID NO: 30 - protein, 298 aa, Micrococcus luteus Otnes 7; identical to SEQ ID NO: 21

SEQ ID NO: 31 - DNA, 1701 bp, Micrococcus luteus Otnes 7
atgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtctgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtggcggcccgcttccaggatccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtcgaggtggtggaccgtcccgcccccgccggccgcctcggacgcctggccgcccgcctgcccaggccgggagcagcccgcggggacgagggccgacgtcgccgcccgggccaggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggagaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggcgagggctggcgggtggaccgcgccggggacgcccaggtggaggccgtggcggaccgggccctcgaccagctggcccgctgggccgggatcccggacctggccgagcgcatcgtggtgcggcgcacctacgggcccggtgacttcgccgcggacgtgcacgcctggcggggttcgctgctgggccccgggcacacgctggcgcagtcggccatgttccgtccctcggtgcgggacgcggacgtggccggcctgatgtacgcgggctcctcggtgcgcccgggcatcggggtgcccatgtgtctgatctccgccgaagtggtccgggacgaactgcgccacgacgcgcgcagggcccggcccgcgggccccggggggagcggcacatga

SEQ ID NO: 32 - protein, 566 aa, Micrococcus luteus Otnes 7
MSARDTALGPRTVVVGGGFAGLATAGLLARDGHRVTLLERGAVLGGRAGRWSEAGFTFDTGPSWYLMPEVIDRWFRLMGTSAAERLDLRRLDPGYRVYFEGHLHEPPVDVRTGHAETLFESLEPGAGRRLRAYLDSASRIYGLAKEHFLYTDFRRPAALAHPDVLRALPALGPQLLGGLRSHVAARFQDPRLRQILGYPAVFLGTSPDRAPAMYHLMSHLDLADGVQYPLGGFAALVDAMAEVVREAGVEIRTGVEATAVEVVDRPAPAGRLGRLAARLPRPGAARGDEGRRRRPGQVTGVAWRSDDGAAGRLDADVVVAAADLHHVQTRLLPPGRRVAESTWDRRDPGPSGVLVCVGVRGSLPQLAHHTLLFTADWEDNFGRIERGEDLAADTSIYVSRTSATDPGVAPEGDENLFILVPAPAEPGWGRGGIRVRDGEGWRVDRAGDAQVEAVADRALDQLARWAGIPDLAERIVVRRTYGPGDFAADVHAWRGSLLGPGHTLAQSAMFRPSVRDADVAGLMYAGSSVRPGIGVPMCLISAEVVRDELRHDARRARPAGPGGSGT

SEQ ID NO: 33 - DNA, 792 bp, Micrococcus luteus Otnes 7
gtgaccccggcccgccccacggtctccgtggtcgtcccggtgctcgacgacgccgagcacctgcgcgtgtgcctcgccctgctggccgcccagagccggcccgcgctggaggtggtggtggtggacaacggctgcgtggacgactcggcggtgctcgcccgcgccgccggcgcgcgggtggtgcacgagccgcgccgcggggtcccggccgcagcggccgccggcctggacgccgcggtcggggagctgctggtgcgctgcgacgccgacacgcggatgcccgcggactggctcgaacggatcgtggcccggttcgacgccgactccgggctcgacgccctcaccgggccggggaccttccacgaccagcccggcctccgggggcgggtgcgggcggcgctctacaccggcgcgtaccgctggggggcgggcgccgcggtggcggccacccccgtctggggctccaactgcgccctgcgcgccgaggcgtggcaggctgtacggacccgcgtccaccgcgagcgcggggacgtgcacgatgacctggacctgtccttccagctggccttggccggccgccggatccggttcgatccggacctgcgggtggaggtcgccgggcgcatcttccactccctgcgccagcgggtgcggcagggccggatggcggtcaccaccctgcaggtcaactgggcccggctgtcccccgggcggcggtggctgcgccgggcggcccgggcacgcccccggccccgctgggggcgtggccccgacggtcagtcccgcgactga

SEQ ID NO: 34 - protein, 263 aa, Micrococcus luteus Otnes 7
VTPARPTVSVVVPVLDDAEHLRVCLALLAAQSRPALEVVVVDNGCVDDSAVLARAAGARVVHEPRRGVPAAAAAGLDAAVGELLVRCDADTRMPADWLERIVARFDADSGLDALTGPGTFHDQPGLRGRVRAALYTGAYRWGAGAAVAATPVWGSNCALRAEAWQAVRTRVHRERGDVHDDLDLSFQLALAGRRIRFDPDLRVEVAGRIFHSLRQRVRQGRMAVTTLQVNWARLSPGRRWLRRAARARPRPRWGRGPDGQSRD

SEQ ID NO: 35 - DNA, 609 bp, Micrococcus luteus Otnes 7
gtgccggtcggcgcggccctcggccgggacgccgcccccacccggacgatcgctgacatgctgcagctgatccccgcagacctgcagcgcgcgctcgacatgatcctcgtcccggtcgcgacggtgcacgcaggatggccgtccgcgacggcgatgctgctcgtgttcggctcccagtggctcacccgctggctcgccccgagcggcgccctggactgggccgcgcaggcggtcctgctgctggccgggtggctgagcgtcatcggcctctacccacgggtgccgtggctggacctgctcgtgcacgccgccgcctccgccgtggtcgcctgtctgacggcactggtggtgggggcatggctccggcgtcgggggaccgaggccgggcaggccgtggcgctgctcggcccgggcctggccggtctggggatcgcggccgccgccgtggccctgggcgtggtgtgggagctggccgaatggcgggggtacacggcggtgacccccgagatcggtgtgggctacacggacaccatcggcgacctcgccgccgatctcgtcggcgccgggatcggcgccgccctcgccgtgcgccgggagcgcacccggtga

SEQ ID NO: 36 - protein, 202 aa, Micrococcus luteus Otnes 7
VPVGAALGRDAAPTRTIADMLQLIPADLQRALDMILVPVATVHAGWPSATAMLLVFGSQWLTRWLAPSGALDWAAQAVLLLAGWLSVIGLYPRVPWLDLLVHAAASAVVACLTALVVGAWLRRRGTEAGQAVALLGPGLAGLGIAAAAVALGVVWELAEWRGYTAVTPEIGVGYTDTIGDLAADLVGAGIGAALAVRRERTR

SEQ ID NO: 37 - DNA, 6606 bp, Micrococcus luteus Otnes 7
atgggtgaagcgaggacgggcggcgaggccgcgctctccggggtgaccgccgagctggacgccgcgctccgacatgccgcggcccaggcacccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgaccgcggccggcgagctcgaagacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgcctcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctgagatggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggctgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtcgctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtctgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtggcggcccgcttccaggatccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtcgaggtggtggaccgtcccgcccccgccggccgcctcggacgcctggccgcccgcctgcccaggccgggagcagcccgcggggacgagggccgacgtcgccgcccgggccaggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggagaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggcgagggctggcgggtggaccgcgccggggacgcccaggtggaggccgtggcggaccgggccctcgaccagctggcccgctgggccgggatcccggacctggccgagcgcatcgtggtgcggcgcacctacgggcccggtgacttcgccgcggacgtgcacgcctggcggggttcgctgctgggccccgggcacacgctggcgcagtcggccatgttccgtccctcggtgcgggacgcggacgtggccggcctgatgtacgcgggctcctcggtgcgcccgggcatcggggtgcccatgtgtctgatctccgccgaagtggtccgggacgaactgcgccacgacgcgcgcagggcccggcccgcgggccccggggggagcggcacatgatccgcaccctcttctgggcgtcccggccggtcagctgggtgaacacggcgtacccgttcgccgccgccgcgatcctgaccggggggctgcccgcgtggctggtggtcctgggcgtcgtgttcttcctcgtgccctacaacctggccatgtacggcatcaatgacgtgttcgacttcgcctcggacctgcgcaacccccgcaaggggggcgtggagggctccgtgctgggcgaccccgcggtgcgccgccgggtgctggtgtggtcggtgctgctgcccgtcccgttcgtggccgtgctcgcgggctggtccgccgtgcggggcgagtgggccgccgtgctggtgctggcggtgagcctgttcgcggtggtggcgtactcctgggcggggctgcggttcaaggagcggcccttcctggacgccgcgacctccgccacccacttcgtctcccccgcggtctacggcctcgtgctggccggggcgacccccacgcccgccctggcggcgctgctgggggccttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccccttgcggcgctgctggccgtgccctacgtggtgaacaccctgcgcttccgccgcatcacggacgccacctcgggcgcggcccaccgcggctggcagctgttcctccccctgaactacgtgaccggcttcctcgtgaccctgctgctgatcgggtgggcgctgacccggggggcggcggcatgatctacctgctggccctgctgggtgtcatcggctgcatgctgctggtggaccggcgcttcgagctgttcctgtggcatcgcccgctcccggcgctgctggtgctggccgccggggtggcctacttcgtcgcctgggacctgtgggggatcgccgaaggcgtgttcctgcaccggcagtcgccctacgtgaccggggtgatgctcgccccccagctgcccctggaggaggggttcttcctgctcttcctcagccagatcacgatggtgctgttcaccggggcgctgcgcctgctgcgcggccggggacgcgacgcccgtgccgcgacgccggccgatccgaccgacggggggagccggtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtggtcgtgccggtcggcgcggccctcggccgggacgccgcccccacccggacgatcgctgacatgctgcagctgatccccgcagacctgcagcgcgcgctcgacatgatcctcgtcccggtcgcgacggtgcacgcaggatggccgtccgcgacggcgatgctgctcgtgttcggctcccagtggctcacccgctggctcgccccgagcggcgccctggactgggccgcgcaggcggtcctgctgctggccgggtggctgagcgtcatcggcctctacccacgggtgccgtggctggacctgctcgtgcacgccgccgcctccgccgtggtcgcctgtctgacggcactggtggtgggggcatggctccggcgtcgggggaccgaggccgggcaggccgtggcgctgctcggcccgggcctggccggtctggggatcgcggccgccgccgtggccctgggcgtggtgtgggagctggccgaatggcgggggtacacggcggtgacccccgagatcggtgtgggctacacggacaccatcggcgacctcgccgccgatctcgtcggcgccgggatcggcgccgccctcgccgtgcgccgggagcgcacccggtgaccccggcccgccccacggtctccgtggtcgtcccggtgctcgacgacgccgagcacctgcgcgtgtgcctcgccctgctggccgcccagagccggccggcgctggaggtggtggtggtggacaacggctgcgtggacgactcggcggtgctcgcccgcgccgccggcgcgcgggtggtgcacgagccgcgccgcggggtcccggccgcggcggccgccggcctggacgccgcggtcggggagctgctggtgcgctgcgacgccgacacgcggatgcccgcggactggctcgaacggatcgtggcccggttcgacgccgactccgggctcgacgccctcaccgggccggggaccttccacgaccagcccggcctccgggggcgggtgcgggcggcgctctacaccggcgcgtaccgctggggggcgggcgccgcggtggcggccacccccgtctggggctccaactgcgccctgcgcgccgaggcgtggcaggctgtacggacccgcgtccaccgcgagcgcggggacgtgcacgatgacctggacctgtccttccagctggccttggccggccgccggatccggttcgatccggacctgcgggtggaggtcgccgggcgcatcttccactccctgcgccagcgggtgcggcagggccggatggcggtcaccaccctgcaggtcaactgggcccggctgtcccccgggcggcggtggctgcgccgggcggcccgggcacgcccccggccccgctgggggcgtggccccgacggtcagtcccgcgactga

SEQ ID NO: 38 - DNA, 28 bp, Artificial sequence
  crtE-F primer
tttttcatatgggtgaagcgaggacggg

SEQ ID NO: 39 - DNA, 34 bp, Artificial sequence
  crtYh-R primer
tttttgcggccgctcagcgatcgtccgggtgggg

SEQ ID NO: 40 - DNA, 33 bp, Artificial sequence
  crtI-R primer
tttttgcggccgctcatgtgccgctccccccgg

SEQ ID NO: 41 - DNA, 28 bp, Artificial sequence
  crtE2-F primer
tttttcatatgatccgcaccctcttctg

SEQ ID NO: 42 - DNA, 31 bp, Artificial sequence
  crtYX-R primer
tttttcctaggagatggccgcgaacatcctg

SEQ ID NO: 43 - DNA, 35 bp, Artificial sequence
  crtYg-R primer
tttttgcggccgctcaccggctcccccggtcggtc

SEQ ID NO: 44 - DNA, 33 bp, Artificial sequence
  crtE2-R primer
tttttgcggccgctcatgccgccgccccccggg

SEQ ID NO: 45 - DNA, 28 bp, Artificial sequence
  crtYg-F primer
tttttcatatgatctacctgctggccct

SEQ ID NO: 46 - DNA, 22 bp, Artificial sequence
  crtE2-F primer
tgaccaacgaccggtagcggag

SEQ ID NO: 47 - DNA, 40 bp, Artificial sequence
  crtE2-i-R primer
cccatccactaaacttaaacatcatgccgccgccccccgg

SEQ ID NO: 48 - DNA, 46 bp, Artificial sequence
  crtYe-i-F primer
tgtttaagtttagtggatgggttgatccctatcatcgatatttcac

SEQ ID NO: 49 - DNA, 37 bp, Artificial sequence
  crtYf-R primer
ttttgcggccgcttttccatcatgactacggcttttc

SEQ ID NO: 50 - DNA, 96 bp, Artificial sequence
  crtYe-F1 primer
tggctatctctagaaaggcctaccccttaggctttatgcaacagaaacaataataatggagtcatgaacatatgatccctatcatcgatatttcac

SEQ ID NO: 51 - DNA, 39 bp, Artificial sequence
  crtYf-R primer
ttttgcggccgcctgatcggataaaagcagagttatatc